(19) United States
(12) Patent Application Publication
(10) Pub. No.: US 2026/0109990 A1
(43) Pub. Date: Apr. 23, 2026

(54) Systems and Methods for Sequential Microfluidic Processing of Cells

(71) Applicant: CellFE, Inc., Alameda, CA (US)

(72) Inventors: Ian Sicher, Alameda, CA (US); Melis Keceli, Alameda, CA (US); Sewoon Han, Albany, CA (US); Chih-Wen Ni, Alameda, CA (US)

(21) Appl. No.: 19/377,662

(22) Filed: Nov. 3, 2025

Related U.S. Application Data
(63) Continuation of application No. PCT/US24/28041, filed on May 6, 2024.
(60) Provisional application No. 63/500,471, filed on May 5, 2023.

(51) Int. Cl. C12N 15/64; B01L 3/00; G01N 15/02; G01N 15/14; G01N 15/149

(52) U.S. Cl. CPC C12N 15/64; B01L 3/502715; B01L 3/502761; G01N 15/149; G01N 2015/0288; G01N 2015/1402

(57) ABSTRACT

Systems and methods for microfluidic sequential processing of a cell or plurality of cells are described herein. The systems may include cell reservoirs, microfluidic transfection devices, a cell sorter, a plurality of valves, and analytical sampling instruments. The system may be used to transfect at least one cell with at least one exogenous molecule.

### CROSS REFERENCE TO RELATED APPLICATIONS

The present application is a continuation of PCT/US2024/028041, filed 2024 May 6, which claims the benefit of U.S. Provisional Application No. 63/500,471, entitled, “SYSTEMS AND METHODS FOR SEQUENTIAL MICROFLUIDIC PROCESSING OF CELLS” filed May 5, 2023, the content of which is hereby incorporated by reference in its entirety.

### FIELD The present disclosure relates generally to microfluidic cell processing methods and systems.

### BACKGROUND

Transfection is the process by which foreign nucleic acids are introduced into eukaryotic cells, thereby enabling modification of the cells'genetic material. Transfection is a powerful tool for cell biology research and for creating cells for diagnostic and therapeutic applications. Foreign nucleic acids that are inserted can include any of a variety of forms of coding or non-coding DNA and RNA. Transfection methodologies are typically identified as either stable or transient depending on whether the foreign nucleic acid is integrated into the target cell genome, resulting in sustained expression even through replication. In transient transfection methods, the foreign nucleic acid is not integrated into the target cell genome is introduced rather as a separate form such as a plasmid or other short length of nucleic acid. Transient transfection results in temporary expression of the nucleic acid which is lost over time as the transfected cells replicate.

For both stable and transient transfection, the foreign nucleic acid is introduced to the cell in a genetic construct known as a vector. The vector may be viral in origin, or a non-viral plasmid vector. Transfection using a viral vector is known as transduction. Viral vectors include adenovirus, adeno-associated virus (AAV) and lentivirus, and are generally considered highly effective and the preferred option for cells that are difficult to transfect. However, transduction and the use of viral vectors have varying limitations. For example, retroviruses like lentivirus are useful only for transfecting cells that are actively dividing cells. Viral vectors also carry increased risks of immunogenicity and pathogenicity. Additionally, viral transfection of multiple different nucleic acid payloads to achieve complex or multiple edits is time-consuming and not readily scalable.

If viral transfection is not being used, transfection requires another means for delivering the foreign nucleic acid in the vector construct to the target cell. Alternative means include chemical and physical or mechanical methods such as electroporation, sonoporation and magnetofection. These methods are generally hard on cells such that cell viability is compromised, and because less efficient in terms of transfection rate, are also not readily scalable. For example, such processes typically require days between separate “back-to-back” transfection events to allow cells sufficient time to recover. Additionally, these methods are not very efficient in generating stably edited cell lines. New transfection methods and systems are needed to address the need for efficient cell transfection, and to allow for scalable transfection of multiple nucleic acid payloads.

### BRIEF SUMMARY

Various aspects of the disclosure are listed below. It will be understood that the aspects listed below may be combined not only as listed, but in other suitable combinations in accordance with the spirit and scope of the invention.

Provided herein is a method for high throughput introduction of a plurality of exogenous molecules into a cell, the method may comprise: combining the cell in an initial fluid medium with a first exogenous molecule to form a first composition; passing the first composition through a microfluidic transfection device, thereby introducing the first exogenous molecule to the cell to form a transfected cell comprising the first exogenous molecule; combining the transfected cell in the initial fluid medium with a second exogenous molecule to form a second composition; passing the second composition through a microfluidic transfection device, thereby introducing the second exogenous molecule to the transfected cell wherein the transfected cell further comprises the second exogenous molecule.

In certain instances, the method of any of the preceding instances may comprise repeating the method with N additional exogenous molecules.

Also provided herein is a method for high throughput introduction of a plurality of exogenous molecules into a cell, the method may comprise: passing the cell through N microfluidic processing cycles wherein N=2 to 10 and wherein each cycle comprises: combining the cell in an initial fluid medium with an exogenous molecule to form a composition; passing the composition through a microfluidic transfection device, thereby introducing the exogenous molecule to the cell to form a transfected cell comprising the exogenous molecule; wherein the exogenous molecule comprises the same or a different molecule for each cycle.

In an instance, the method of any of the preceding instances may further comprise performing the method on a plurality of cells wherein the cells are a homogenous population.

In another instance, the method of any of the preceding instances may further comprise before combining the transfected cell comprising the first exogenous molecule with a second exogenous molecule in the initial fluid medium, and optionally holding the cell for a hold period.

In an instance, the hold period may be selected from a period between about 0.1 seconds to about 60 minutes.

In certain instances, each exogenous molecule may be independently selected from gene editing materials, nanoparticles, protein, antigens, amino-acids, viruses or viral components, DNA and related material, RNA and related material, lipids and related material, small molecules, and/or salts.

In certain instances, the first and/or the second exogenous molecule may comprise a nucleotide sequence encoding an amino acid sequence, the method further comprising maintaining the cell or cell(s) in holding period for a time and conditions sufficient for expression of the first and/or second exogenous molecule.

In certain instances, the transfection parameters may be selected from device gap, supply pressure, supply flow rate and/or cell velocity, ridge number, channel width, height, length, gap width, height, length, ridge spacing, ridge angle, processing buffer constituents, cell type, cell source, and/or number of parallelized channels.

Also provided herein is a population of cells comprising at least one cell transfected with at least the first exogenous molecule and at least one cell transfected with at least the second exogenous molecule by the methods.

In certain instances, about 20% to about 40% of the cells may be successfully transfected in each transfection.

In certain instances, the sequential nature of the transfection events may produce a high cell viability and high rate of transfection.

In certain instances, about 50% to about 90% of the cells may remain viable after the first transfection and the second transfection.

In certain instances, the method may further comprise optionally observing a hold period after each cycle.

Also provided herein is a method for high throughput introduction of an exogenous molecule into cells in a population of cells which are heterogeneous, the method may comprise: obtaining or having obtained the population of cells sorted into at least a first sub-population and a second sub-population of cells, wherein the first sub-population and second sub-population differ in size, cell stiffness, adhesiveness, and/or FACS characteristics; combining the first sub-population of cells in an initial fluid medium with the exogenous molecule to form a first composition; combining the second sub-population of cells in the initial fluid medium with the exogenous molecule thereby forming a second composition; passing the first composition through a microfluidic transfection device using a first set of process parameters, thereby introducing the exogenous molecule into the cells in the first sub-population to form a first population of transfected cells comprising the exogenous molecule; passing the second composition through a microfluidic transfection device using a second set of process parameters, thereby introducing the exogenous molecule to the cells in the second sub-population to form a second population of transfected cells comprising the exogenous molecule; optionally observing a hold period.

In certain instances, the method may comprise a microfluidic processing cycle, and the method may comprise passing the cell through N microfluidic processing cycles wherein N=2 to 10.

In certain instances, obtaining the population of cells sorted into at least a first sub-population and a second sub-population of cells may comprise passing the heterogenous cell population through a cell sorter, thereby producing at least a first sub-population comprising cells each having a cell diameter below a first diameter cut-off value, and a second sub-population of larger cells each having a diameter above the first diameter cut-off value and below a second diameter cut-off value.

In certain instances, obtaining the population of cells sorted into at least the first sub-population and the second sub-population may comprise passing the heterogenous cell population through a cell sorter, thereby producing at least the first sub-population comprising cells having a first set of FACS characteristics, and a second sub-population of comprising cells having a second set of FACS characteristics.

In certain instances, the first set and second set of FACS characteristics may comprise different phenotypes.

In certain instances, the first set and second set of FACS characteristics may comprise differing receptor types.

In certain instances, the first set and second set of FACS characteristics may comprise differing frequencies.

In certain instances, obtaining the population of cells sorted into at least a first sub-population and a second sub-population of cells may comprise passing the heterogenous cell population through a cell sorter, thereby producing at least a first sub-population comprising cells each having a cell stiffness below a first stiffness cut-off value, and a second sub-population comprising cells each having a stiffness above the first stiffness cut-off value and below a second stiffness cut-off value.

In certain instances, obtaining the population of cells sorted into at least a first sub-population and a second sub-population may comprise obtaining the cells sorted into N additional sub-populations by sorting cells into groups defined by additional ranges of cell diameters.

In certain instances, the method may further comprise combining the first and the second up to the Nth sub-populations of transfected cells to form a heterogenous cell product.

In certain instances, the heterogenous cell population may comprise whole blood, wherein sorting the cells comprises sorting the cells into at least a first sub-population having an average cell diameter of 10-12 μm (neutrophils), a second sub-population having an average cell diameter of 12-15 μm (lymphocytes), and a third sub-population having an average cell diameter of 15-30 μm (monocytes).

In certain instances, the exogenous molecule may be selected from gene editing materials, nanoparticles, protein, antigens, amino-acids, viruses or viral components, DNA and related material, RNA and related material, lipids and related material, small molecules, and/or salts.

In certain instances, the exogenous molecule may comprise a nucleotide sequence encoding an amino acid sequence of interest, the method further comprising maintaining the combined cell populations in a holding period for a time and conditions sufficient for the cells to express the one or more nucleotide sequences.

Further provided herein is a population of cells comprising at least one cell transfected with the exogenous molecule by any of the methods.

In certain instances, the hold period may be about 0.1 seconds to about 60 minutes.

Further provided here is a method for high throughput introduction of a plurality of exogenous molecules to a plurality of cells comprising a heterogenous cell population, the method may comprise: obtaining the population of cells sorted into at least a first sub-population and a second sub-population of cells, wherein the first sub-population and second sub-population differ in size and/or cell stiffness and/or FACS characteristics; combining a first exogenous molecule with the first sub-population of cells in an initial fluid medium to form a first cell composition; passing the first cell composition through a microfluidic transfection device, thereby introducing the first exogenous molecule into the cells to form a first population of transfected cells comprising the first exogenous molecule; combining a second exogenous and optionally the first exogenous molecule with the second sub-population of cells in the initial fluid medium to form a second cell composition; passing the second cell composition through a microfluidic transfection device, thereby introducing the second exogenous molecule and optionally the first exogenous molecule into the cells to form a second population of transfected cells comprising the second exogenous molecule and optionally the first exogenous molecule; and optionally repeating the steps n times with the first, second, and/or n additional exogenous molecules to form N additional populations of transfected cells, each additional population transfected with the first exogenous molecule, the second exogenous molecule, and/or an additional exogenous molecule. The additional exogenous molecules may be the same exogenous molecule or a different exogenous molecule from the first exogenous molecule and the second exogenous molecule.

In certain instances, the method may further comprise optionally holding the cells for a holding period before optionally repeating the steps.

In certain instances, the FACS characteristics may be one or more of phenotype, receptor type, and/or frequency.

In certain instances, the method may further comprise tuning the microfluidic transfection device prior to passing the cell compositions through the microfluidic transfection device.

In certain instances, the microfluidic transfection device may be tuned based on the sub-population size, cell stiffness, or FACS characteristics.

In certain instances, tuning the microfluidic transfection device may comprise increasing or decreasing a gap size of the microfluidic transfection device. In certain instances, tuning the microfluidic transfection device may comprise increasing or decreasing a driving fluid pressure of the microfluidic transfection device.

Further provided herein is a method for high throughput introduction of a plurality of exogenous molecules to a plurality of cells comprising a heterogenous cell population, the method may comprise: passing the cell through N microfluidic processing cycles wherein N=2 to 20 and wherein each cycle comprises: obtaining the population of cells sorted into at least a first sub-population and a second sub-population of cells, wherein the first sub-population and second sub-population differ in size, cell stiffness, and/or FACS characteristics; combining a first exogenous molecule with the first sub-population of cells in an initial fluid medium to form a first cell composition; passing the first cell composition through a microfluidic transfection device, thereby introducing the first exogenous molecule into the cells to form a first population of transfected cells comprising the first exogenous molecule; combining a second exogenous and optionally the first exogenous molecule with the second sub-population of cells in the initial fluid medium to form a second cell composition; passing the second cell composition through a microfluidic transfection device, thereby introducing the second exogenous molecule and optionally the first exogenous molecule into the cells to form a second population of transfected cells comprising the second exogenous molecule and optionally the first exogenous molecule; and optionally observing a hold period after each complete cycle.

In certain instances, N=20.

In certain instances, obtaining the cells sorted may comprise passing the heterogeneous population of cells in the initial fluid medium through a cell sorter, wherein the cell sorter sorts the cells according to differences in average cell diameters, differences in cell stiffness, and/or differences in FACS characteristics.

In certain instances, the heterogenous cell population may comprise whole blood, wherein sorting the cells comprises sorting the cells into at least a first sub-population having an average cell diameter of 10-12 μm (neutrophils), a second sub-population having an average cell diameter of 12-15 μm (lymphocytes), and a third sub-population having an average cell diameter of 15-30 μm (monocytes).

In certain instances, the method may further comprise combining the first and the second populations of transfected cells, and optionally combining up to N additional population(s), to form a heterogenous cell product.

In certain instances, each exogenous molecule may be independently selected from gene editing materials, nanoparticles, protein, antigens, amino-acids, viruses or viral components, DNA and related material, RNA and related material, lipids and related material, small molecules, and/or salts.

In certain instances, any one or more of the exogenous molecules each independently may comprise a nucleotide sequence encoding an amino acid sequence of interest, the method further comprising maintaining the combined cell populations in a holding period for a time and conditions sufficient for the cells to express the one or more nucleotide sequences.

Further provided herein is a population of cells comprising at least one cell transfected with at least the first exogenous molecule and at least one cell transfected with at least the second exogenous molecule by the method.

In certain instances, the population may comprise at least one cell transfected with at least one of each exogenous molecule up to the Nth exogenous molecule.

In certain instances, the microfluidic parameters may comprise device gap, supply pressure, supply flow rate and/or cell velocity, ridge number, channel width, height, length, gap width, height, ridge angle, length, ridge spacing, processing buffer constituents, cell type, cell source, temperature, and/or number of parallelized channels.

Also provided herein is a method for high throughput introduction of a plurality of exogenous molecules to a plurality of cells, the method may comprise: combining the cells in an initial fluid medium with a first exogenous molecule to form a first composition; passing the first composition through a first transfection component of a microfluidic cassette, thereby introducing the first exogenous molecule to the cells to form a population of transfected cells comprising the first exogenous molecule; collecting the population of transfected cells in a holding chamber of the microfluidic cassette; combining the transfected cells with a second exogenous molecule to form a second composition in the holding chamber; passing the second composition through a second transfection component of the microfluidic cassette, thereby introducing the second exogenous molecule to the population of transfected cells wherein the population of transfected cells further comprises the second exogenous molecule; optionally repeating the steps N times with N additional exogenous molecules to form N additional populations of transfected cells, each additional population transfected with an additional exogenous molecule.

In certain instances, the cells may be sorted prior to being combined in the initial fluid medium.

In certain instances, the cells may be sorted by size, cell stiffness, and/or FACS characteristics.

In certain instances, the method may further comprise observing a hold period before optionally repeating the method.

In certain instances, the plurality of cells may be heterogenous or homogenous.

In certain instances, the method may further comprise passing the transfected cells into a collection reservoir.

In certain instances, the first exogenous molecule and the second exogenous molecule may be independently selected from gene editing materials, nanoparticles, protein, antigens, amino-acids, viruses or viral components, DNA and related material, RNA and related material, lipids and related material, small molecules, salts, and/or different cell types.

In certain instances, the second exogenous molecule may be provided to the holding chamber by a payload reservoir and a valve in fluid communication with the holding chamber.

In certain instances, the method may further comprise, combining a second population of cells with the transfected cells in the holding chamber to form a third population of cells comprising the transfected cells and the second population of cells; providing a third exogenous molecule to the third population of cells to form a third composition; passing the third composition through a third transfection component of the microfluidic cassette, thereby introducing the third exogenous molecule to the third population of cells wherein the third population of cells further comprises the third exogenous molecule.

In certain instances, the second population of cells may be a sorted population of cells.

In certain instances, the second population of cells may have a size below a first diameter cut-off or above the first diameter cut-off.

In certain instances, the second population of cells may have a cell stiffness below a first stiffness cut-off or above a first stiffness cut-off.

In certain instances, the second population of cells may be sorted by certain FACS characteristics such as phenotype, receptor type, and/or frequency.

In certain instances, the first and/or the second exogenous molecule may comprise a nucleotide sequence encoding an amino acid sequence, the method further comprising maintaining the cell or cell(s) in a holding period for a time and conditions sufficient for expression of the first and/or second exogenous molecule.

Also provided herein is a method for high throughput introduction of a plurality of exogenous molecules to a plurality of cells comprising a cell population, the method may comprise: placing the population of cells in a holding chamber of a microfluidic cassette from an initial reservoir; combining the cells with a first exogenous molecule to form a first composition wherein the first exogenous molecule is provided by a port in fluid communication with the holding chamber and a payload reservoir; passing the first composition through a first transfection component of the microfluidic cassette, thereby introducing the first exogenous molecule to the cell population to form a population of transfected cells comprising the first exogenous molecule; returning the transfected cells to the holding chamber; supplying the payload reservoir with a second exogenous molecule; combining the transfected cells with the second exogenous molecule to form a second composition; passing the second composition through a second transfection component of the microfluidic cassette, thereby introducing the second exogenous molecule to the transfected cells wherein the transfected cells further comprise the second exogenous molecule; optionally repeating the steps N times with N additional exogenous molecules to form N additional populations of transfected cells using N additional transfection components, each additional population transfected with an additional exogenous molecule. The additional exogenous molecules may be the same or different from the first exogenous molecule and the second exogenous molecule.

In certain instances, the transfected cells may be removed from the microfluidic cassette via a collection reservoir.

In certain instances, transfected cells may be removed from the microfluidic cassette directly via the initial reservoir.

In certain instances, the method may comprise combining the first exogenous molecule with the cell population in an initial fluid medium prior to entering the microfluidic cassette.

In certain instances, N is 20.

In certain instances, the first exogenous molecule, the second exogenous molecule and up to the Nth exogenous molecules may be independently selected from gene editing materials, nanoparticles, protein, antigens, amino-acids, viruses or viral components, DNA and related material, RNA and related material, lipids and related material, small molecules, and/or salts.

In certain instances, any one or more of the first exogenous molecule, the second exogenous molecule and N exogenous molecule(s) each may independently include a nucleotide sequence encoding an amino acid sequence of interest, the method further comprising maintaining the combined cell populations in a holding period for a time and conditions sufficient for the cells to express the one or more nucleotide sequences.

Further provided herein is a population of cells comprising at least one cell transfected with at least the first exogenous molecule and at least one cell transfected with at least the second exogenous molecule by the method.

In certain instances, the population of cells can include at least one cell transfected with at least one of each exogenous molecule up to the Nth exogenous molecule.

In certain instances, the method may include supplying a pressure to move the cells between the holding chamber and the first transfection component and/or the second transfection component.

In certain instances, the method may include supplying a flow rate to the cells using a flow rate supply.

In certain instances, the pressure may be about 5 psi to about 100 psi.

In certain instances, the pressure may be about 20 psi.

In certain instances, the pressure may be about 40 psi.

In certain instances, a first valve may control a flow of cells from the holding chamber to the first transfection component.

In certain instances, a second valve may control a flow of cells from the first transfection component to the holding chamber.

In certain instances, a third valve may control a flow of cells from the holding chamber to the second transfection component.

In certain instances, a fourth valve may control a flow of cells from the second transfection component to the holding chamber.

Further provided here is a method for high throughput introduction of at least one exogenous molecules into a plurality of cells comprising a cell population, the method may comprise: combining a first exogenous molecule with the cell population to form a first composition; placing the first composition in a first reservoir of a microfluidic consumable; providing a pressure to the first reservoir through a first pressure supply to pass the first composition through a microfluidic transfection component to a second reservoir, thereby introducing the first exogenous molecule into the cells to form a first population of transfected cells comprising the first exogenous molecule; optionally observing a holding period; optionally combining a second exogenous molecule with the first population of transfected cells in the second reservoir; providing a pressure to the second reservoir through a second pressure supply to pass the transfected cells through the microfluidic transfection component to the first reservoir; and optionally repeating N times with N additional exogenous molecules to form N additional populations of transfected cells, each additional population transfected with the same or an additional exogenous molecule. The additional exogenous molecule may be the same as or different from the first exogenous molecule and the second exogenous molecule.

In certain instances, the first reservoir may have a first payload reservoir for injecting an Nth exogenous molecule.

In certain instances, the second reservoir may have a second payload reservoir for injecting an Nth exogenous molecule.

In certain instances, the microfluidic consumable may have a plurality of transfection components.

In certain instances, the pressure supplied by the first pressure supply and/or the second pressure supply may be about 5 psi to about 100 psi.

In certain instances, the pressure may be supplied by a pressure instrument.

In certain instances, the pressure instrument may be a pressure supply having multiple regulators.

In certain instances, N is 20.

In certain instances, the first exogenous molecule, the second exogenous molecule and up to the Nth exogenous molecules may be each independently selected from gene editing materials, nanoparticles, protein, antigens, amino-acids, viruses or viral components, DNA and related material, RNA and related material, lipids and related material, small molecules, and/or salts.

In certain instances, any one or more of the first exogenous molecule, the second exogenous molecule and N exogenous molecule(s) each independently may include a nucleotide sequence encoding an amino acid sequence of interest, the method further including maintaining the combined cell populations in a holding period for a time and conditions sufficient for the cells to express the one or more nucleotide sequences.

Further provided herein is a population of cells that includes at least one cell transfected with at least the first exogenous molecule and at least one cell transfected with at least the second exogenous molecule.

In certain instances, the population of cells may include at least one cell transfected with at least one of each exogenous molecule up to the Nth exogenous molecule.

In certain instances, passing the transfected cells through the microfluidic transfection component to the first reservoir without a second exogenous molecule increases transfection efficiency.

Further provided herein is a method for high throughput introduction of a plurality of exogenous molecules to a plurality of cells comprising a cell population, the method comprising: placing the cell population in a first reservoir of a microfluidic consumable; combining a first exogenous molecule with the cells to form a first composition in the first reservoir through a first payload reservoir in fluid communication with the first reservoir; providing a pressure to the first reservoir through a first pressure supply to pass the first composition through a first transfection component to a second reservoir, thereby introducing the first exogenous molecule to the cell population to form a population of transfected cells comprising the first exogenous molecule; collecting a sample of the transfected cells through a sample removal port in fluid communication with the second reservoir; combining a second exogenous molecule with the transfected cells to form a second composition in the second reservoir through a second payload reservoir in fluid communication with the second reservoir; providing a pressure to the second reservoir through a second pressure supply to pass the second composition through a second transfection component to a third reservoir, thereby introducing the second exogenous molecule to the transfected cells wherein the transfected cells further comprise the second exogenous molecule; and optionally repeating N times with N additional exogenous molecules to form N additional populations of transfected cells, each additional population transfected with an additional exogenous molecule.

In certain instances, the method may further comprise collecting a sample of the transfected cells through a sample removal port in fluid communication with the third reservoir.

In certain instances, the sample may be analyzed by an analytical instrument.

In certain instances, the analytical instrument may be a flow cytometer, a cell counter, a next generation sequencing instrument, or other analytical instrument capable of analyzing the transfected cells. The method may further comprise holding the cells for a holding period in each reservoir between transfections to allow for analysis of the sample.

In certain instances, N=20.

In certain instances, the first exogenous molecule, the second exogenous molecule and up to the Nth exogenous molecules may each be independently selected from gene editing materials, nanoparticles, protein, antigens, amino-acids, viruses or viral components, DNA and related material, RNA and related material, lipids and related material, small molecules, and/or salts.

In certain instances, any one or more of the first exogenous molecule, the second exogenous molecule and N exogenous molecule(s) may each independently comprise a nucleotide sequence encoding an amino acid sequence of interest, the method further including maintaining the combined cell populations in a holding period for a time and conditions sufficient for the cells to express the one or more nucleotide sequences.

Further provided herein is population of cells comprising at least one cell transfected with at least the first exogenous molecule and at least one cell transfected with at least the second exogenous molecule by the method of any of the preceding instances.

In certain instances, the population of cells may comprise at least one cell transfected with at least one of each exogenous molecule up to the Nth exogenous molecule.

In certain instances, there may be no intervening cell culture step between a first transfection and a second transfection.

In certain instances, an expansion step may not be necessary.

In certain instances, the methods may be automated.

In certain instances, the methods may be conducted in a self-contained environment.

In certain instances, the cells may be transfected sequentially without being removed from the device, cassette, or consumable.

In certain instances, each additional transfection event may be completing using one of the previously selected exogenous molecules.

Further provided herein is a system for transfecting a plurality of cells with at least one exogenous molecule which may include: an initial fluid medium; a first microfluidic transfection device; a second microfluidic transfection device; and a first holding chamber; wherein the initial fluid medium is in fluid communication with the first microfluidic transfection device, the second microfluidic transfection device, and the first holding chamber.

In certain instances, the system may further include a first payload reservoir in fluid communication with the initial fluid medium, where the first payload reservoir is configured to deliver a first exogenous molecule to a plurality of cells in the initial fluid medium to form a first composition.

In certain instances, the first microfluidic transfection device may include: an inlet configured to receive the first composition; a transfection component configured to transfect the plurality of cells with the first exogenous molecule, thereby producing a first population of transfected cells; and an outlet configured to return the first population of transfected cells to the initial fluid medium.

In certain instances, the system of any of the preceding instances may further include a second payload reservoir in fluid communication with the initial fluid medium, where the second payload reservoir is configured to deliver a second exogenous molecule to the first population of transfected cells in the initial fluid medium to form a second composition.

In certain instances, the second microfluidic transfection device may include an inlet configured to receive the second composition; a transfection component configured to transfect the first population of transfected cells with the second exogenous molecule, thereby producing a second population of transfected cells; and an outlet configured to return the second population of transfected cells to the initial fluid medium.

In certain instances, the system of any of the preceding instances may further include a pressure supply in fluid communication with the initial fluid medium and configured to supply a pressure to move the cells through the initial fluid medium.

In certain instances, the system of any of the preceding instances may further include N microfluidic transfection devices; N payload reservoirs; and N holding chambers.

In certain instances, N may be 3 to 30.

In certain instances, the system may further include a flow rate supply.

In certain instances, the flow rate supply may be configured to supply a cell velocity of about 1 mm/s to about 20 mm/s.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the first microfluidic transfection device may have a channel cross-sectional dimension of about 300 μm and a channel length of 1000 μm.

In certain instances, the second microfluidic transfection device may have a channel cross-sectional dimension of about 300 μm and a channel length of 1000 μm.

In certain instances, the first microfluidic transfection device may have about 1 ridge to about 5 ridges.

In certain instances, the second microfluidic transfection device may have about 1 ridge to about 5 ridges.

Further provided herein is a system for transfecting a plurality of cells including: an initial fluid medium; a cell sorter in fluid communication with the initial fluid medium; a payload reservoir in fluid communication with the initial fluid medium; a first microfluidic transfection device in fluid communication with the initial fluid medium; a second microfluidic transfection device in fluid communication with the initial fluid medium; wherein the first microfluidic transfection device and the second microfluidic transfection device are arranged in parallel and configured to transfect cells simultaneously.

In certain instances, the cell sorter may be configured to sort the plurality of cells into at least a first sub-population and a second sub-population based on size, cell stiffness, FACS characteristics and/or other physical properties.

In certain instances, the initial fluid medium may comprise a first flow path and a second flow path, wherein the first flow path and the second flow path are configured in parallel.

In certain instances, the first flow path may be operable to deliver the first sub-population of cells to the first microfluidic transfection device.

In certain instances, the second flow path may be operable to deliver the second sub-population of cells to the second microfluidic transfection device.

In certain instances, the payload reservoir may be configured to inject the first sub-population and the second sub-population of cells with an exogenous molecule in the first flow path and the second flow path, thereby producing a first composition and a second composition.

In certain instances, the first microfluidic transfection device may include: an inlet configured to receive the first composition from the first flow path; a transfection component configured to transfect the first composition with the exogenous molecule, thereby producing a first population of transfected cells; and an outlet configured to return the first population of transfected cells to the initial fluid medium.

In certain instances, the second microfluidic transfection device may include: an inlet configured to receive the second composition from the second flow path; a transfection component configured to transfect the second composition with the exogenous molecule, thereby producing a second population of transfected cells; an outlet configured to return the second population of transfected cells to the initial fluid medium.

In certain instances, the system may include a collection reservoir in fluid communication with the initial fluid medium and the cell sorter. The collection reservoir may be configured to collect the first population of transfected cells and the second population of transfected cells.

In certain instances, the cell sorter may be configured to sort the first population of transfected cells and the second population of transfected cells into at least a third sub-population and a fourth sub-population based on size, cell stiffness, FACS characteristics, and/or other physical properties.

In certain instances, the system may further include a flow rate supply configured to supply a cell velocity of about 5 mm/s to about 20 mm/s.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the first microfluidic transfection device may have a channel cross-sectional dimension of 300 μm and a channel length of 2000 μm.

In certain instances, the second microfluidic transfection device may have a channel cross-sectional dimension of 300 μm and a channel length of 2000 μm.

In certain instances, the first microfluidic transfection device may have about 1 ridge to about 5 ridges.

In certain instances, the second microfluidic transfection device may have about 1 ridge to about 5 ridges.

Further provided herein is a system for transfecting a plurality of cells with at least one exogenous molecule comprising: an initial fluid medium; a cell sorter in fluid communication with the initial fluid medium; a first microfluidic transfection device in fluid communication with the initial fluid medium; a second microfluidic transfection device in fluid communication with the initial fluid medium; a first payload reservoir in fluid communication with the initial fluid medium; and a second payload reservoir in fluid communication with the initial fluid medium.

In certain instances, the cell sorter may be configured to sort the plurality of cells into two or more cell sub-populations based on size, cell stiffness, or FACS characteristics.

In certain instances, the plurality of cells may be sorted into a first sub-population and a second sub-population.

In certain instances, the first payload reservoir may be configured to inject the first sub-population of cells with a first exogenous molecule in the initial fluid medium forming a first composition.

In certain instances, the first microfluidic transfection device may comprise: an inlet to receive the first composition from the initial fluid medium; a transfection component configured to transfect the first sub-population of cells with the first exogenous molecule, thereby producing a first population of transfected cells; and an outlet to return the first population of transfected cells to the initial fluid medium.

In certain instances, the second sub-population of cells may be combined with the first population of transfected cells in the initial fluid medium to form a second composition.

In certain instances, the second payload reservoir may be configured to inject the second composition with a second exogenous molecule.

In certain instances, the second microfluidic transfection device may comprise: an inlet configured to receive the second composition; a transfection component configured to transfect the second composition with the second exogenous molecule, thereby producing a second population of transfected cells; and an outlet configured to return the second population of transfected cells to the initial fluid medium.

In certain instances, the system may further include a pressure supply in fluid communication with the initial fluid medium and configured to supply a pressure to move the cells through the initial fluid medium.

In certain instances, the system may further include N microfluidic transfection devices and N payload reservoirs. N may be 3 to 30.

In certain instances, the system may further include a flow rate supply.

In certain instances, the flow rate supply is configured to supply a cell velocity of about 1 mm/s to about 20 mm/s.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the first microfluidic transfection device may have a channel cross-sectional dimension of 300 μm and a channel length of 2000 μm.

In certain instances, the second microfluidic transfection device may have a channel cross-sectional dimension of 300 μm and a channel length of 2000 μm.

In certain instances, the first microfluidic transfection device may have about 1 ridge to about 5 ridges.

In certain instances, the second microfluidic transfection device may have about 1 ridge to about 5 ridges.

Further provided herein is a system for transfecting a plurality of cells with at least one exogenous molecule comprising: an initial fluid medium; a first payload reservoir in fluid communication with the initial fluid medium; a microfluidic cassette in fluid communication with the initial fluid medium comprising: a first microfluidic transfection device; a holding chamber; a second microfluidic transfection device; a second payload reservoir in fluid communication with the microfluidic cassette; and a collection reservoir in fluid communication with the microfluidic cassette.

In certain instances, the microfluidic cassette may comprise a first valve located between the first microfluidic transfection device and the holding chamber.

In certain instances, the microfluidic cassette may comprise a second valve located between the second payload reservoir and the holding chamber.

In certain instances, the first payload reservoir may be configured to inject a first exogenous molecule into the plurality of cells, thereby producing a first composition.

In certain instances, the first microfluidic transfection device may include: an inlet configured to receive the first composition from the initial fluid medium; a transfection component configured to transfect the first composition with the first exogenous molecule, thereby producing a first population of transfected cells; and an outlet configured to move the first population of transfected cells to the holding chamber.

In certain instances, the second payload reservoir may be configured to inject a second exogenous molecule into the first population of transfected cells in the holding chamber, thereby producing a second composition.

In certain instances, the second microfluidic transfection device may comprise: an inlet configured to receive the second composition from the holding chamber; a transfection component configured to transfect the second composition with the second exogenous molecule, thereby producing a second population of transfected cells; and an outlet configured to move the second population of transfected cells to the collection reservoir.

In certain instances, the system may include a pressure supply in fluid communication with the initial fluid medium and configured to supply a pressure to move the cells through the initial fluid medium, the microfluidic cassette, and to the collection reservoir.

In certain instances, the collection reservoir may be configured to collect the second population of transfected cells from the second microfluidic transfection device.

In certain instances, the system may include a cell sorter.

In certain instances, the cell sorter may be configured to sort the cells into two or more sub-populations by size, cell stiffness, FACS characteristics, and/or other physical characteristics.

In certain instances, the second payload reservoir may be configured to inject a second sub-population of cells into the holding chamber.

In certain instances, the system may further include a flow rate supply.

In certain instances, the flow rate supply is configured to supply a cell velocity of about 1 mm/s to about 20 mm/s.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the first microfluidic transfection device may have a channel cross-sectional dimension of 300 μm and a channel length of 2000 μm.

In certain instances, the second microfluidic transfection device may have a channel cross-sectional dimension of 300 μm and a channel length of 2000 μm.

In certain instances, the first microfluidic transfection device may have about 1 ridge to about 5 ridges.

In certain instances, the second microfluidic transfection device may have about 1 ridge to about 5 ridges.

Further provided herein is a system for transfecting a plurality of cells with at least one exogenous molecule including: an initial fluid medium; a first payload reservoir in fluid communication with the initial fluid medium; a microfluidic cassette in fluid communication with the initial fluid medium and the first payload reservoir comprising: a holding chamber having a payload reservoir inlet; a first microfluidic transfection device; a second microfluidic transfection device; a plurality of valves in fluid communication with the holding chamber and the first and second microfluidic transfection devices: a second payload reservoir in fluid communication with the payload reservoir inlet; and a collection reservoir.

In certain instances, the plurality of valves may include: a first valve located between the first payload reservoir and the holding chamber; a second valve located between the second payload reservoir and the payload reservoir inlet; a third valve located between the holding chamber and the first microfluidic transfection device; a fourth valve located between the first microfluidic transfection device and the holding chamber; a fifth valve located between the holding chamber and the second microfluidic transfection device; a sixth valve located between the second microfluidic transfection device and the holding chamber; and a seventh valve located between the holding chamber and the collection reservoir.

In certain instances, the first payload reservoir may be configured to inject a first exogenous molecule into the plurality of cells, thereby producing a first composition.

In certain instances, the first microfluidic transfection device may include: an inlet configured to receive the first composition from the initial fluid medium; a transfection component configured to transfect the first composition with the first exogenous molecule, thereby producing a first population of transfected cells; and an outlet configured to move the first population of transfected cells to the holding chamber.

In certain instances, the second payload reservoir may be configured to inject a second exogenous molecule into the first population of transfected cells in the holding chamber, thereby producing a second composition.

In certain instances, the second microfluidic transfection device may include: an inlet configured to receive the second composition from the holding chamber; a transfection component configured to transfect the second composition with the second exogenous molecule, thereby producing a second population of transfected cells; and an outlet configured to move the second population of transfected cells to the holding chamber.

In certain instances, the system may further include a pressure supply in fluid communication with the initial fluid medium and configured to supply a pressure to move the cells through the initial fluid medium, the microfluidic cassette, and to the collection reservoir.

In certain instances, the collection reservoir may be configured to collect the second population of transfected cells.

In certain instances, the system of any of the preceding instances may further comprise a cell sorter.

In certain instances, the cell sorter may be configured to sort the cells into two or more sub-populations by size, cell stiffness, FACS characteristics, and/or other physical characteristics.

In certain instances, the second payload reservoir may be configured to inject a second sub-population of cells into the holding chamber.

In certain instances, the system of any of the preceding instances may further comprise a flow rate supply.

In certain instances, the flow rate supply may be configured to supply a cell velocity of about 1 mm/s to about 20 mm/s.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the first microfluidic transfection device may have a channel cross-sectional dimension of about 300 μm and a channel length of 2000 μm.

In certain instances, the second microfluidic transfection device may have a channel about 300μm and a channel length of 2000 μm.

In certain instances, the first microfluidic transfection device may have about 1 ridge to about 5 ridges.

In certain instances, the second microfluidic transfection device may have about 1 ridge to about 5 ridges.

In certain instances, the microfluidic cassette may further comprise N microfluidic transfection devices.

In certain instances, N may be 3 to 30.

Further provided herein is a microfluidic consumable system for transfecting a plurality of cells with at least one exogenous molecule comprising: a first reservoir; a second reservoir; and a microfluidic transfection device in fluid communication with the first reservoir and the second reservoir.

In certain instances, the first reservoir may have an inlet for injecting a composition comprising the plurality of cells and an exogenous molecule.

In certain instances, the second reservoir may have an outlet for removing the plurality of cells.

In certain instances, the system may further include a first payload reservoir in fluid communication with the inlet of the first reservoir.

In certain instances, the system may further include a second payload reservoir in fluid communication with the second reservoir.

In certain instances, the system o may further include a first pressure supply in fluid communication with the first reservoir, wherein the first pressure supply is configured to supply a pressure to move the composition from the first reservoir through the microfluidic transfection device to the second reservoir.

In certain instances, when the composition passes through the microfluidic transfection device the plurality of cells may be transfected with the exogenous molecule, thereby producing a population of transfected cells.

In certain instances, the system may further include a flow rate supply. The flow rate supply may be configured to provide a cell velocity of about 1 mm/s to about 20 mm/s.

In certain instances, the microfluidic transfection device may have a channel cross-sectional dimension of 300 μm and a channel length of 2000 μm.

In certain instances, the microfluidic transfection device may have about 1 ridge to about 5 ridges.

In certain instances, the system can further include N is 2 to 30.

In certain instances, the system may further include a second pressure supply in fluid communication with the second reservoir, wherein the second pressure supply is configured to supply a pressure to the second reservoir to more the population of transfected cells through the microfluidic transfection device to the first reservoir.

Further provided here is a microfluidic consumable system including: an initial fluid medium; a first reservoir in fluid communication with the initial fluid medium; a first microfluidic transfection device in fluid communication with the first reservoir; a second reservoir in fluid communication with the first microfluidic transfection device; a second microfluidic transfection device in fluid communication with the second reservoir; a third reservoir in fluid communication with the second microfluidic transfection device; and a third microfluidic transfection device in fluid communication with the third reservoir.

In certain instances, the first reservoir may include: a first payload reservoir; a first pressure supply; and a first sample removal port.

In certain instances, the first reservoir may contain a plurality of cells.

In certain instances, the first payload reservoir may be configured to deliver a first exogenous molecule to the plurality of cells in the first reservoir, producing a first composition.

In certain instances, the first pressure supply may provide a pressure to move the first composition from the first reservoir through the first microfluidic transfection device to the second reservoir.

In certain instances, the first microfluidic transfection device may include: an inlet configured to receive the first composition from the first reservoir; a transfection component configured to transfect the first composition with the first exogenous molecule, thereby producing a first population of transfected cells; and an outlet configured to move the first population of transfected cells to the second reservoir.

In certain instances, the second reservoir may comprise: a second payload reservoir; a second pressure supply; and a second sample removal port.

In certain instances, the second reservoir may contain the first population of transfected cells.

In certain instances, the second payload reservoir may be configured to deliver a second exogenous molecule to the first population of transfected cells in the second reservoir, producing a second composition.

In certain instances, the second pressure supply may provide a pressure to move the second composition from the second reservoir through the second microfluidic transfection device to the third reservoir.

In certain instances, the second microfluidic transfection device may include: an inlet configured to receive the second composition from the second reservoir; a transfection component configured to transfect the second composition with the second exogenous molecule, thereby producing a second population of transfected cells; and an outlet configured to move the first population of transfected cells to the third reservoir.

In certain instances, the second sample removal port may be configured to remove a sample of the first population of transfected cells.

In certain instances, the third reservoir may comprise: a third payload reservoir; a third pressure supply; and a third sample removal port.

In certain instances, the third reservoir may contain the second population of transfected cells.

In certain instances, the third payload reservoir may be configured to deliver a third exogenous molecule to the second population of transfected cells in the third reservoir, producing a third composition.

In certain instances, the third pressure supply may provide a pressure to move the third composition from the third reservoir through the third microfluidic transfection device.

In certain instances, the third microfluidic transfection device may include: an inlet configured to receive the third composition from the third reservoir; a transfection component configured to transfect the third composition with the third exogenous molecule, thereby producing a third population of transfected cells; and an outlet.

In certain instances, the third sample removal port may be configured to remove a sample of the second population of transfected cells.

In certain instances, the first microfluidic transfection device may have a gap of about 5.4 μm to about 5.6 μm.

In certain instances, the second microfluidic transfection device may have a gap of about 5.4 μm to about 5.6 μm.

In certain instances, the third microfluidic transfection device may have a gap of about 5.4 μm to about 5.6 μm.

In certain instances, the first microfluidic transfection device may have a channel cross-sectional dimension of about 300 μm and a channel length of 2000 μm.

In certain instances, the second microfluidic transfection device may have a channel cross-sectional dimension of about 300 μm and a channel length of 2000 μm.

In certain instances, the third microfluidic transfection device may have a channel cross-sectional dimension of about 300 μm and a channel length of 2000 μm.

In certain instances, the first microfluidic transfection device may have about 1 ridge to about 5 ridges.

In certain instances, the second microfluidic transfection device may have about 5 ridges.

In certain instances, the third microfluidic transfection device may have about 1 ridge to about 5 ridges.

In certain instances, the first microfluidic transfection device may have an optimized ridge number, channel width, channel height, channel length, gap width, gap size, gap length, ridge spacing, and number of parallelized channels for the transfection of cells with the first exogenous molecule.

In certain instances, the second microfluidic transfection device may have an optimized ridge number, channel width, channel height, channel length, gap width, gap size, gap length, ridge spacing, and number of parallelized channels for the transfection of cells with the second exogenous molecule.

In certain instances, the Nth microfluidic transfection device may have an optimized ridge number, channel width, channel height, channel length, gap width, gap size, gap length, ridge spacing, and number of parallelized channels for the transfection of cells with the Nth exogenous molecule.

In certain instances, the system may be self-contained and automated.

In certain instances, the system may be operable to transfect the plurality of cells with two or more exogenous molecules within one hour.

In certain instances, the flow rate supply may supply an optimal cell velocity for the transfection of the plurality of cells with the first exogenous molecule.

In certain instances, the flow rate supply may supply an optimal cell velocity for the transfection of the plurality of cells with the second exogenous molecule.

In certain instances, the flow rate supply may supply an optimal cell velocity for the transfection of the plurality of cells with the Nth exogenous molecule.

In certain instances, a ridge angle of the microfluidic transfection device is optimized.

In certain instances, the cells are sorted by adhesiveness.

Other aspects and iterations of the invention are described more thoroughly below.

### BRIEF DESCRIPTION OF THE DRAWINGS

FIG. 1A is an example of a microfluidic system.

FIG. 1B is an example of a sequential microfluidic system.

FIG. 2 is a flow chart of a sequential microfluidic process in one example.

FIG. 3A is a side view of a microfluidic transfection device in one example.

FIG. 3B is a side view of a microfluidic transfection device in one example.

FIG. 4 is an example of a sequential microfluidic system.

FIG. 5 is a flow chart of a sequential microfluidic process in one example.

FIG. 6 is an example of a sequential microfluidic system.

FIG. 7 is a flow chart of a sequential microfluidic process in one example.

FIG. 8 is an example of a sequential microfluidic system.

FIG. 9 is a flow chart of a sequential microfluidic process in one example.

FIG. 10A is an example of a sequential microfluidic system.

FIG. 10B is an example of a microfluidic cassette.

FIG. 10C is an example of a microfluidic cassette.

FIG. 11 is a flow chart of a sequential microfluidic process in one example.

FIG. 12A is a top view of an example of a sequential microfluidic system.

FIG. 12B is a side view of an example sequential microfluidic system.

FIG. 12C is a view of an example of a sequential microfluidic system.

FIG. 12D is a side view of an example of a sequential microfluidic system.

FIG. 12E is a top view of an example of a sequential microfluidic system.

FIG. 13 is a flow chart of a sequential microfluidic process in one example.

FIG. 14 is an example of a sequential microfluidic system.

FIG. 15 is a flow chart of a sequential microfluidic process in one example.

FIG. 16A is a graph showing viability optimization results for a sequential microfluidic process.

FIG. 16B is a graph showing transfection optimization results for a sequential microfluidic process.

FIG. 17A is a bar graph showing TCR Knockout percentage for a sequential microfluidic process.

FIG. 17B is a bar graph showing viability percentage for a sequential microfluidic process.

FIG. 18 is a bar graph showing TCR Knockout percentage results for a sequential microfluidic process.

FIG. 19 is a bar graph showing normalized viability percentage for a sequential microfluidic process.

FIG. 20 is a top view of a microfluidic transfection device in one example.

FIG. 21A is a bar graph of percentage of raw viability at D0 (day 0) and D5 (day 5) post-transfection with 48 hours of T cell activation.

FIG. 21B is a bar graph of percentage of raw viability before and after sequential editing.

FIG. 21C is a bar graph of percentage of raw viability at D0 and D5 post-transfection in T-cells sequentially edited with 24 hours of T cell activation.

FIG. 21D is a bar graph of percentage of raw viability at D0 and D5 post-transfection in T-cells, 6 hours after T cell activation in cells with single delivery, co-delivery, sequential delivery, and No Device (ND) controls.

FIG. 22A is a bar graph of percentage of knock-outs in T-cells with or without sequential editing with 48 hours of T cell activation.

FIG. 22B is a bar graph of percentage of knock-outs in T-cells with or without sequential editing with 24 hours of T cell activation.

FIG. 22C is a bar graph of percentage of knock-outs in T-cells 6 hours after T cell activation in cells with single delivery, co-delivery, sequential delivery, and No Device (ND) controls.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the aspects described herein. The drawings are not necessarily to scale and the proportions of certain parts may be exaggerated to better illustrate details and features of the present disclosure.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “coupled” is defined as connected, whether directly or indirectly through intervening components, and is not necessarily limited to physical connections. The connection can be such that the objects are permanently connected or releasably connected. The term “substantially” is defined to be essentially conforming to the particular dimension, shape or other word that substantially modifies, such that the component need not be exact. For example, “substantially cylindrical” means that the object resembles a cylinder but can have one or more deviations from a true cylinder.

The terms “attached” and “connected” are used interchangeably in this disclosure. The terms “attached”and “connected”mean to be connected.

The terms “comprising,” “including” and “having” are used interchangeably in this disclosure. The terms “comprising,” “including” and “having” mean to include, but not necessarily be limited to the things so described.

The terms “payload reservoir” and “payload injector” are used interchangeably in this disclosure.

Provided herein are systems and methods for sequential microfluidic processing of cells. The system is configured for sequential processing of a cell or cells through a microfluidic transfection device to transfect the cell or cells with at least one exogenous molecule. The system decreases the amount of time required to introduce multiple different exogenous molecules into a cell (e.g., the amount of time required to transfect a cell with multiple different exogenous molecules as compared to known transfection methods). The system provides not only a shorter processing time for multiple transfection events, but also higher cell viability and successful transfection events compared to the systems known in the art. The systems and the methods may provide a high throughput introduction of one or more exogenous molecules into a cell or a plurality of cells. It will be appreciated that the systems and methods described herein may be rearranged or combined. The components of any of the systems or methods herein may function substantially the same as the components of any of the other systems or methods disclosed herein. In some examples, the systems described herein can allow for sequential transfection of multiple exogenous molecules into a cell or cells without removing the cell or cells from the system. In some examples, the systems may be self-contained.

The systems and methods provided herein allow materials (e.g., exogenous molecules) to be delivered intracellularly using back-to-back transfection events with minimal impact to cell viability over several events and retention of high delivery efficiency to the cells. Typically, performing this process back-to-back with existing technologies results in low cell viability and/or low delivery efficiency. Using the systems and methods described herein cells can be transfected back-to-back and retain sufficient cell numbers and sufficient cell health to allow for rapidly following one transfection event with a second transfection event. Using the systems and methods herein the resting time between transfection events can be less than one hour, and in some cases, there may be no resting time at all. The sequential nature of the systems and methods described herein produce a high cell viability and a high rate of transfection. The systems and methods herein are configured for sequential microfluidic processing and therefore may not require an expansion. The systems may be self-contained and the methods using the systems may be conducted in the self-contained environment of the systems.

A system may include an initial fluid medium, at least one reservoir, at least one payload reservoir, at least one microfluidic transfection device, and at least one holding chamber. The system may be configured to allow for transfection of at least one exogenous molecule into a cell or a population of cells. The at least one reservoir may be in fluid communication with the initial fluid medium. The at least one reservoir my contain a cell or a plurality of cells. In an example, the cells contained in the at least one reservoir may be homogenous. The cell or cells in the at least one reservoir may be provided to the initial fluid medium via a movement mechanism (e.g., a pump, a flow rate supply, etc.).

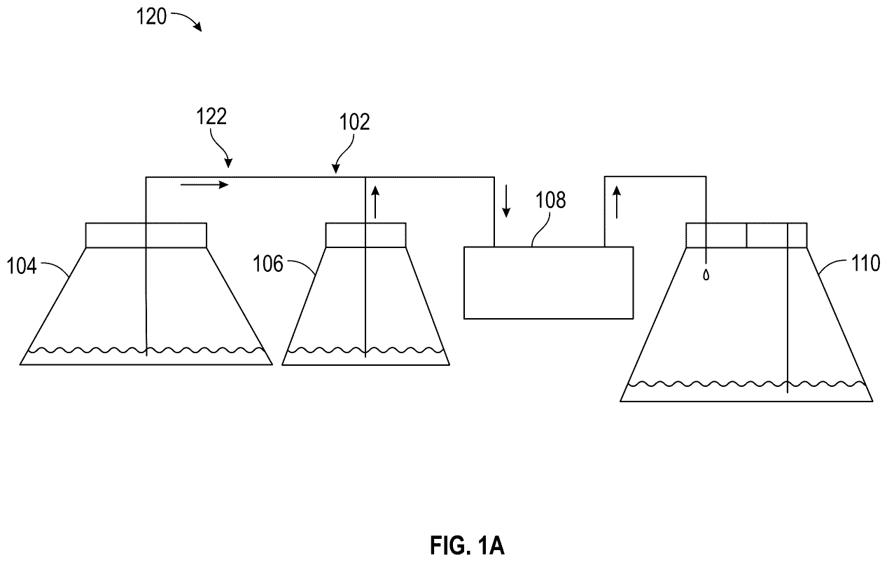

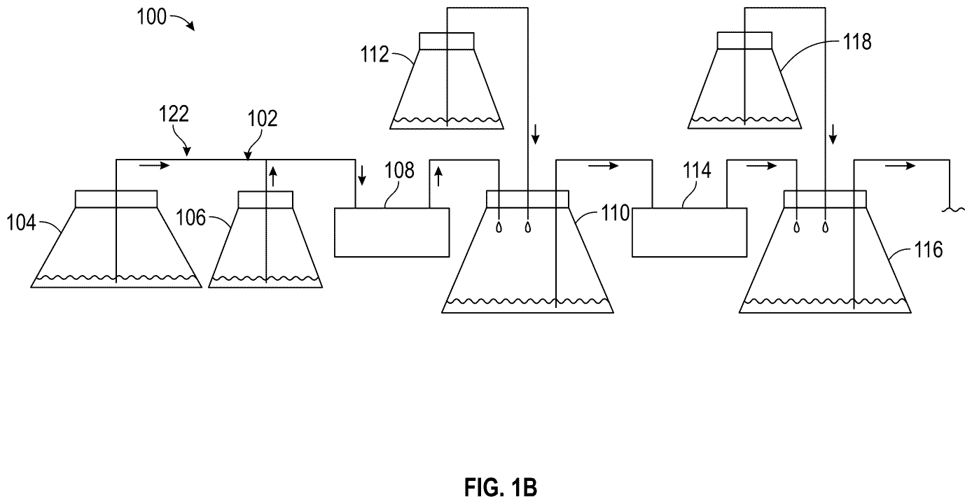

As illustrated in FIGS. 1A, 1B, 4, 6, 8, and 10A, the systems 120, 100, 400, 600, 800, and 1000 may include an initial fluid medium 102. The initial fluid medium 102 may be contained within a tubing 122 or conduits connecting the various components of the systems 120, 100, 400, 600, 800, and 1000. In some examples, when the initial fluid medium 102 is contained in the tubing 122 or conduits, the initial fluid medium 102 may also include the tubing 122. Reference to the initial fluid medium 102 may include the initial fluid medium 102 and the tubing 122.

FIG. 1A illustrates a system 120 for high throughput introduction of a plurality of exogenous molecules into a cell or population of cells. The system 120 is a basic system that may be used as a component or module in some or all of the other systems described herein. In some examples, the two or more of the systems 120 can be connected in series (e.g., sequentially) or in parallel (e.g., for concurrent transfection) to form one or more of the systems described herein. The system 120 may also be used multiple times to effect multiple transfection events in a cell or population of cells. As illustrated in FIG. 1A, the system 120 may include an initial fluid medium 102, at least one reservoir (e.g., first reservoir 104), at least one payload reservoir (e.g., first payload reservoir 106), at least one microfluidic transfection device (e.g., first microfluidic transfection device 108), and at least one holding chamber (e.g., a first holding chamber 110). The system 100 may be operable to transfect a cell or a plurality of cells with at least one exogenous molecule. The system 120, as illustrated in FIG. 1A, is configured to deliver at least one exogenous molecule to a cell or plurality of cells.

As illustrated in FIG. 1A, the first reservoir 104 may be in fluid communication with the initial fluid medium 102. The first reservoir 104 may contain a cell or a plurality of cells. In an example, the cells may be homogenous. The first reservoir 104 may be operable to provide the plurality of cells to the initial fluid medium 102. In some examples, the initial fluid medium 102 may be held within tubing in the system 120. In some examples, the initial fluid medium 102 may be in the first reservoir 104 and be moved through a tubing 122 of the system 120. In some examples, the initial fluid medium 102 may be provided to the tubing 122 by an initial fluid medium reservoir (similar to first reservoir 104). All the components of system 120 may be in fluid communication with the tubing 122.

In some examples, the initial fluid medium 102 may be a culture medium. In some examples, the culture medium may include at least one of interleukin (IL)-2, IL-7 and IL-15, or any combination thereof. It will be appreciated that the initial fluid medium 102 may be used in any of the systems and methods described herein.

Cells may be moved from the first reservoir 104 to the initial fluid medium 102 by applying a flow rate using a flow rate supply (e.g., pump or pressure supply) to provide a cell velocity to the cells. A flow rate supply as referred to herein means a device operable to cause flow of liquid, solid, or gas in a pipe, tube, or medium.

In some examples, any of the cells or may be resting or inactivated cells, such as resting T-cells or NK cells. Cells that may be made by any of the disclosed methods, using any of the disclosed methods or systems, or which comprise any of the disclosed systems, or cells contained within any microfluidic device as described herein may comprise CAR-T or CAR-NK cell or cells. The cells may be any kind of cell where transfection of one or more exogenous molecules is desired.

As illustrated in FIG. 1A, the first payload reservoir 106 may be in fluid communication with the initial fluid medium 102. In some examples, the first payload reservoir 106 is downstream of the first reservoir 104. The first payload reservoir 106 may contain one or more exogenous molecules to be transfected into a cell. The first payload reservoir 106 may inject the one or more exogenous molecules into the initial fluid medium 102 by using a flow rate supply (e.g., pump, etc.). When the one or more exogenous molecules are delivered to the initial fluid medium 102, the one or more exogenous molecules and the cell or cells may form a first composition.

The one or more exogenous molecules may include gene editing materials, nanoparticles, proteins, antigens, amino-acids, viruses or viral components, DNA and related material, RNA and related material, lipids and related materials, small molecules, salts, and any combination thereof. In an example, the one or more exogenous molecules may comprise a nucleotide sequence encoding an amino acid sequence of interest. It will be appreciated that the types of exogenous molecules may be used in all of the systems and methods described herein. In some examples, the one or more exogenous molecules may be sgRNA and CRISPR associated protein 9 (Cas9). In some examples, the one or more exogenous molecules may be a lyophilized gRNA. In other examples, the one or more exogenous molecules may be Cas9. In some examples, the one or more exogenous molecules may include two or more exogenous molecules comprising a vector. The types of exogenous molecules described in this paragraph may be used as a first, second, third, fourth, fifth, or Nth exogenous molecule to be transfected into a cell or a plurality of cells using any of the systems or methods described herein.

In some aspects, the one or more exogenous molecules may be used for a knock-out gene editing therapy. In an example, the one or more exogenous molecules may be used for a knock-in gene therapy. In other examples, multiple exogenous molecules may be used for a knock-in or a knock-out gene editing therapy. In some examples, a first exogenous molecule and a second exogenous molecule may be used in combination to perform a gene editing therapy. In some examples, the first exogenous molecule may be a plasmid configured to insert an attP sequence in a desired locus of a cell. The second exogenous molecule may be a plasmid configured to perform an attB docking sequence. For example, a plasmid may comprise units to express the nCas9-M-MLV fusion protein along with expression modules of pegRNA and gRNA to perform a CRISPR prime editing for inserting an attP site in the desired locus. A second plasmid may comprise PhiC31 (serine integrase). A third plasmid may be an insertion cassette containing CAR construct and an attB docking sequence.

In another aspect, a first exogenous molecule and a second exogenous molecule may be configured to produce a gene edit on a cell. In some examples, the first exogenous molecule may prepare the cell or cells for insertion of the second exogenous molecule. The second exogenous molecule may provide the gene edit.

As illustrated in FIG. 1A, the first composition may be passed through a first microfluidic transfection device 108. The first microfluidic transfection device 108 may be in fluid communication with the initial fluid medium 102 and be downstream of the first reservoir 104 and the first payload reservoir 106. The first microfluidic transfection device 108 may include an inlet configured to receive the first composition, a transfection component configured to transfect the cell or cells with the first exogenous molecule, thereby producing a first population of transfected cells or a first transfected cell, and an outlet configured to return the first population of transfected cells to the initial fluid medium. In an example, the first population of transfected cells or first transfected cell may comprise the first exogenous molecule.

The cell or cells may have a successful transfection percentage of about 20% to about 25%, about 25% to about 30%, about 30% to about 35%, or about 35% to about 40% after being transfected using the first transfection device. In an example, the cell or cells may have a successful transfection percentage of about 20% to about 40%. In some examples, the transfection success rate may be about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the first microfluidic transfection device 108. In an example, about 50% to about 90% of the cells may remain viable after being transfected using the first microfluidic transfection device 108. As referred to herein, viable means alive. In some examples, viable may mean that the cells are capable of successfully undergoing another transfection event.

The transfection components of the microfluidic transfection devices disclosed herein may have various transfection parameters optimized for transfecting the cell or cells (e.g., to produce a high viability rate and a high successful transfection rate). In an example, the transfection parameters for optimized transfection of the cell or cells with an exogenous molecule may be device gap, supply pressure, supply flow rate, cell velocity, ridge number, channel width, channel height, channel length, gap width, gap size (e.g., height), gap length, ridge spacing, ridge angle, temperature, processing buffer constituents, cell type, cell source, and/or number of parallelized channels. Optimization, types, and functions of microfluidic transfection devices are described in U.S. Publication No. 2021/0292700A1, which is incorporated herein by reference in its entirety. It will be appreciated that the optimization or tuning of the microfluidic transfection device described in this paragraph may be applied to any of the microfluidic transfection devices in this system 120 or in any other system or method described herein. Each microfluidic transfection device described in this system 120, or any other system or method described herein, may have optimized or tuned transfection parameters for a transfection event depending on the size, stiffness, adhesiveness, FACS characteristics, other physical characteristics (e.g., color, shape, etc.), or necessary or desired compression of the cell or cells to be transfected. FACS characteristics refers to fluorescent activated cell sorting. FACS characteristics allow cells to be sorted according to each cell's fluorescent intensity.

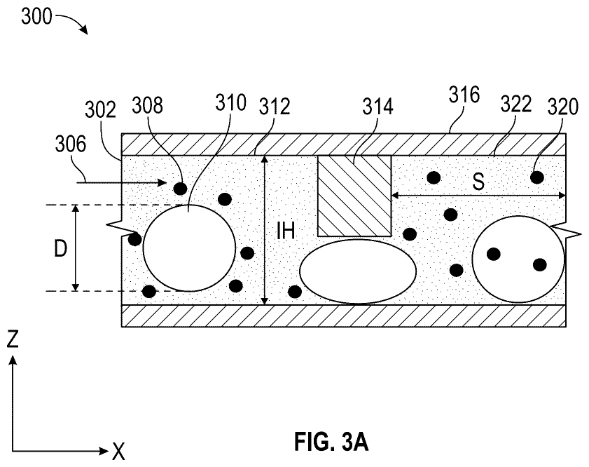

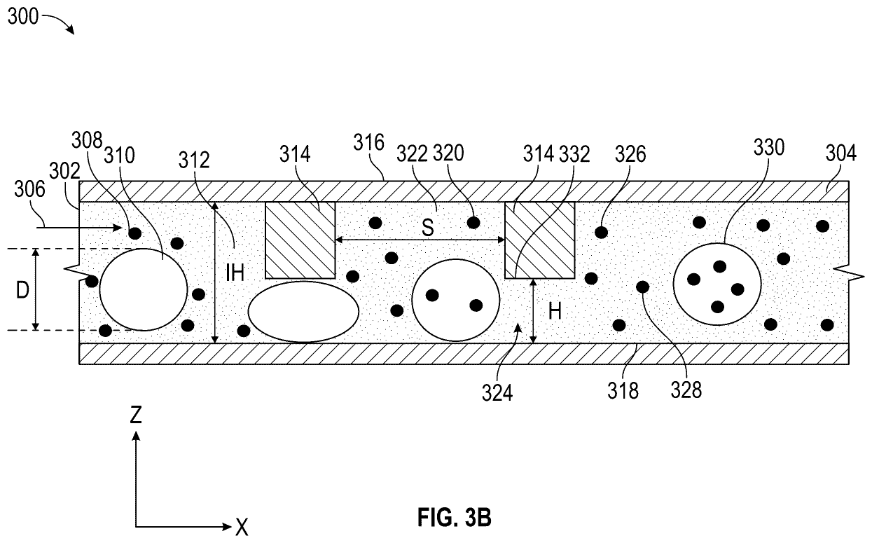

FIGS. 3A-B and 20 illustrate example microfluidic transfection devices 300 (e.g., first microfluidic transfection device 108 and/or second microfluidic transfection device 114). The microfluidic transfection device may have one or more inlets 302 and one or more outlets 304. The arrow 306 illustrates the direction of the flow of cells 310, exogenous molecules 308, and/or compositions through the microfluidic transfection device 300. The microfluidic transfection device 300 may include a liquid media 312. The microfluidic transfection device 300 may include one or more ridges 314 operable to squeeze the cells, open the cell membrane, and allow one or more exogenous molecules to be placed within the cells. For example, the microfluidic transfection device 300 may have one ridge as illustrated in FIG. 3A. In an example, the microfluidic transfection device may have two ridges as illustrated in FIG. 3B. The microfluidic transfection device 300 may have a first wall 316 (e.g., top wall) having a first interior surface 326 and a second wall 318 (e.g., bottom wall) having a second interior surface 328. The microfluidic transfection device 300 may have a third wall 336 (e.g., first side wall) and a fourth wall 338 (e.g., second side wall). The microfluidic transfection device 300 may have a recovery space 320. The one or more ridges 314 may have a ridge spacing 322. The one or more ridges 314 may have a ridge surface 332. The microfluidic transfection device 300 may have a gap 324. The microfluidic transfection device 300 may have cell counters 334. After passing through the components of the microfluidic transfection device 300, a transfected cell 330 may be produced.

In some examples, the microfluidic transfection device 300 may be made of elastomers such as polydimethylsiloxane (PDMS). In some examples, the microfluidic transfection device 300 may be made of PDMS, silicon, glass, metal, resin, epoxy resin, and/or combinations thereof. In some examples, the microfluidic transfection device 300 may be made of any suitable material.

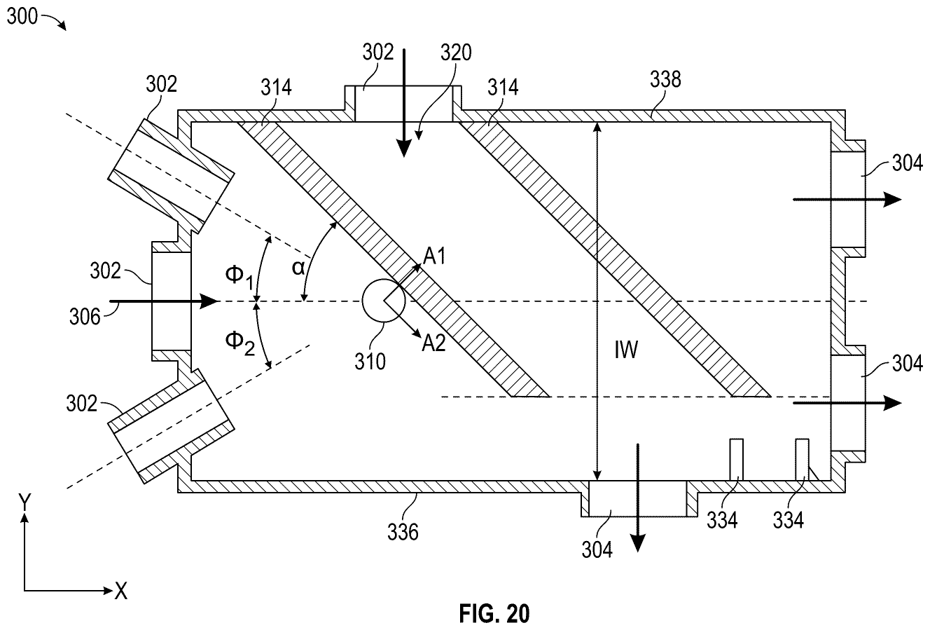

In an aspect, the first wall 316, second wall 318, third wall 336, and fourth wall 338 may enclose the microfluidic transfection device 300 to have an interior height (IH) and an interior width (IW), as illustrated, for example, in FIGS. 3B and 20.

The inlets 302 may be located at different locations and at different angles, as illustrated, for example, in FIG. 20. In some examples, the angle (φ1 and/or φ2) may be between 20 degrees and 80 degrees. In some examples, the angle (φ1 and/or φ2) may be about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, about 45 degrees to about 50 degrees, about 50 degrees to about 55 degrees, about 55 degrees to about 60 degrees, about 60 degrees to about 65 degrees, about 65 degrees to about 70 degrees, about 70 degrees to about 75 degrees, about 75 degrees to about 80 degrees, about 80 degrees to about 85 degrees, or about 85 degrees to about 90 degrees. In some examples, one or more inlets 302 may be located at a point after one of the one or more ridges 314, as illustrated, for example, in FIG. 20.

The gap 324 of the transfection device 300 (e.g., 108, 114) may be the distance between the surface 332 of the one or more ridges 314 and the second interior surface 328. The gap 324 may be smaller than the height of the cell or cells to be transfected. The transfection component of the microfluidic transfection device 300 may have a gap 324 of about 5.4 μm to about 5.6 μm. In a further example, the transfection component may have a gap 324 of about 0.5 μm to about 1 μm, about 1 μm to about 1.5 μm, about 1.5 μm to about 2 μm, about 2 μm to about 2.5 μm, about 2.5 μm to about 3 μm, about 3 μm to about 3.5 μm, about 4 μm to about 4.5 μm, about 4.5 μm to about 5 μm, about 5 μm to about 5.5 μm, about 5.5 μm to about 6 μm, about 6 μm to about 6.5 μm, about 6.5 μm to about 7 μm, about 7 μm to about 7.5 μm, about 7.5 μm to about 8 μm, about 8 μm to about 8.5 μm, about 8.5 μm to about 9 μm, about 9 μm to about 9.5 μm, or about 9.5 μm to about 10 μm. In another example, the gap 324 may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, or about 90 μm to about 100 μm. In an example, the gap 324 may be optimized based on the type and size of the cell, the compression needed or desired, and other characteristics of microfluidic transfection. By optimizing the gap 324 of the transfection component of the microfluidic transfection device 300, the viability percentage of cells after transfection and the successful transfection percentage of cells may be optimized. In an example, the gap size may be optimized by decreasing the gap size to a value lower than the size of the cell or cells to be transfected (e.g., smaller cells have smaller gap sizes and larger cells have larger gap sizes). In an example, the optimized gap 324 for a T-cell may be about 5.3 μm to about 5.6 μm.

As illustrated in FIGS. 3A-B and 20, cells may be transfected by flowing through the gap 324 along flow path A1. Abnormal cells (e.g., cells with low compressibility) may flow along flow path A2 and out of the microfluidic transfection device 300 without being transfected. By flowing abnormal cells out of the microfluidic transfection device 300 without allowing them to pass through the gap, clogging or other negative effects may be prevented.

The transfection component of the microfluidic transfection device 300 may have a gap width and gap length (e.g., length of the ridge surface 332) optimized for transfection of the cell or cells with the first exogenous molecule depending on the size, stiffness, FACS characteristics, other physical cell properties of the cell or cells or the compression necessary or desired for the transfection event. In an example, the optimal gap width and gap length may depend on the type of cell and the type of exogenous molecule. In an example, the gap width may be about 5 μm and the gap length may be about 5 μm. In some examples, the gap width may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, or about 700 μm to about 800 μm, or about 800 μm to about 1000 μm, or about 1000 μm to about 5000 μm, or about 5000 μm to about 10000 μm. In some examples, the gap length (e.g., ridge surface 332 length) may be about 1 μm to about 2 μm, about 2 μm to about 3 μm, about 3 μm to about 4 μm, about 4 μm to about 5 μm, about 5 μm to about 6 μm, about 6 μm to about 7 μm, about 7 μm to about 8 μm, about 8 μm to about 9 μm, about 9 μm to about 10 μm, about 10 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, or about 700 μm to about 800 μm. In an example, for a T-cell the optimal gap width may be 12 μm and the optimal gap length may be 12 μm.

The transfection component of the microfluidic transfection device 300 may have a channel cross-sectional dimension (e.g., diameter) and channel length optimized for transfection of the cell or cells with the first exogenous molecule depending on the size, stiffness, FACS characteristics, or other physical properties of the cell or cells. FIGS. 3A-3B illustrate a single channel microfluidic transfection device 300. In an example, the optimal channel cross-sectional dimension and channel length may depend on the type of cell. In an example, the channel cross-section dimension may be about 1 μm to about 10 μm, about 10 μm to about 20 μm, about 20 μm to about 30 μm, about 30 μm to about 40 μm, about 40 μm to about 50 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700, about 700 μm to about 800 μm, about 800 μm to about 900 μm, about 900 μm to about 1000 μm, about 1000 to about 1250 μm, about 1250 μm to about 1500 μm, about 1500 μm to about 1750 μm, or about 1750 μm to about 2000 μm. The channel length may be about 1 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 2000 μm, about 2000 μm to about 3000 μm, about 3000 μm to about 4000 μm, about 4000 μm to about 5000 μm, or more.

The transfection component of the microfluidic transfection device 300 may have an optimal number of ridges to transfect the cell or cells with an exogenous molecule depending on the size, stiffness, FACS characteristics, and/or other physical properties of the cell or cells. In an example, the first transfection component may have about 1, 2, 3, 4, 5, 6, 7, 8, 9, 10, 11, 12, 13, 14, 15, 16, or more ridges. For example, T-cells may exhibit the same or better results for 1 ridge (e.g., as illustrated in FIG. 3A) as compared to 5. There are other processing metrics aside from cell metrics (i.e., viability and transfection) to using 1 ridge, including reduced running pressure and pressure requirements overall, higher throughput, etc. However, for other cell types different ridge numbers may perform better. For example, early testing of peripheral blood mononuclear cells shows generally better results with 5 ridges than 1 ridge.

In an aspect, the one or more ridges 314 may be rectangular and extend into the microfluidic transfection device 300 at a 90 degree angle from the first interior surface 326. As illustrated in FIG. 20, the one or more ridges 314 may extend into the microfluidic transfection device 300 at an angle α (ridge angle). In some examples, the angle α may be about a 10 degree angle, about a 20 degree angle, about a 30 degree angle, about a 40 degree angle, about a 50 degree angle, about a 60 degree angle, about a 70 degree angle, about an 80 degree angle, or about a 90 degree angle from the first interior surface 326. In other examples, the one or more ridges 314 may be rounded, pointed, or have any other shape. In some examples, the one or more ridges 314 may be trapezoidal, triangular, or ellipsoidal. In some examples, the one or more ridges 314 may have an angled upper surface. For example, the one or more ridges 314 may increase in dimension (e.g., height within the channel) from a front surface (e.g., proximal inlet 302) to a back surface (e.g., distal to inlet 302). In some examples, the one or more ridges 314 may have rounded edges. For example, the one or more ridges 314 may have rounded edges rather than the square edges shown in FIGS. 3A-3B. The rounded edges may have a radius of about 0.05 micrometers (μm) to about 0.1 μm, about 0.1 μm to about 0.5 μm, about 0.5 μm to about 1.0 μm, about 1.0 μm to about 1.5 μm, about 1.5 μm to about 2.0 μm, about 2.0 micrometers to about 2.5 μm, about 2.5 μm to about 3.0 μm, about 3.0 μm to about 3.5 μm, about 3.5 μm to about 4.0 μm, about 4.0 μm to about 4.5 μm, about 4.5 μm to about 5.0 μm, about 5.0 μm to about 5.5 μm, about 5.5 μm to about 6.0 μm, about 6.0 μm to about 6.5 μm, about 6.5 μm to about 7.0 μm, about 7.0 μm to about 7.5 μm, about 7.5 μm to about 8.0 μm, about 8.0 μm to about 8.5 μm, about 8.5 μm to about 9.0 μm, about 9.0 μm to about 9.5 μm, about 9.5 μm to about 10.0 μm, or more.

In an aspect, the one or more ridges 314 may have a surface roughness at the ridge surface 332. In some examples, the one or more ridges 314 may have a surface roughness of about 1 nm to about 10 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1000 nm, about 1000 nm to about 1500 nm, or about 1500 nm to about 2000 nm.

The transfection component of the microfluidic transfection device 300 may have optimal ridge spacing 322 for transfection of the cell or cells with an exogenous molecule depending on the size, stiffness, FACS characteristics, or other physical properties of the cell or cells. In an example, the optimal ridge spacing 322 may depend on the type of cell and the type of first exogenous molecule. In an example, the optimal ridge spacing may be about 100 μm. In some examples, the ridge spacing 322 may be about 10 μm to about 50 μm, about 50 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 5000 μm, or about 5000 μm to about 50000 μm.

The system may include processing buffer constituents. In an example, the processing buffer constituents may be selected based on cell type (e.g., size, stiffness, FACS characteristics, and/or other physical characteristics of the cell or cells). The processing buffer constituents may be selected from buffer constituents configured for microfluidic transfection devices. For example, a basal cell culture medium may be the processing buffer. In other examples, buffer constituents may be chosen based on whether the cells are primary cells (i.e., from a patient) or from cell lines (i.e., immortal test cell types).

In another aspect, the microfluidic transfection device 300 may have multiple parallelized channels for transfecting the cell or cells with an exogenous molecule. In some examples, parallelized channels may mean that the multiple channels are stacked on top of one another. For example, FIGS. 3A-3B show a single channel of the microfluidic transfection device 300. Multiple channels may be parallelized (e.g., stacked on top of each other and/or horizontally placed next to one another). In some examples, the microfluidic transfection device 300 may have about 2, 3, 4, 5, 6, 7, 8, 9, 10, 11, 12, 13, 14, 15, 16, 17, 18, 19, 20 parallelized channels. In other examples, the microfluidic transfection device 300 may have about 1 to about 10 parallelized channels, about 10 to about 100 parallelized channels, about 100 to about 500 parallelized channels, about 500 to about 1000 parallelized channels, about 1000 to about 5000 parallelized channels, about 5000 to about 15000 parallelized channels, or more parallelized channels.

In further aspects, the transfection parameters of the microfluidic transfection device 300 may affect each other. For example, increasing the number of ridges may increase the necessary pressure. In other examples, some transfection parameters may require other transfection parameters to be adjusted to optimize the microfluidic transfection device. It will be appreciated that the microfluidic transfection device 300 can be used in all the systems and methods described herein.

Referring back to FIG. 1A, the first microfluidic transfection device 108 may have optimized transfection parameters for transfecting the cell or cells with the one or more exogenous molecules.

After the cell or cells have been transfected with the one or more exogenous molecules by the first microfluidic transfection device 108 (e.g., first transfection event), the transfected cell or cells are returned to the initial fluid medium 102 via the outlet in the first microfluidic transfection device 108. As illustrated in FIG. 1A, the cell or cells may flow through the initial fluid medium 102 to a first holding chamber 110. The first holding chamber 110 may be downstream from the first microfluidic transfection device. In some examples, the cell or cells are pushed through the first microfluidic transfection device 108 via a flow rate supply (e.g., pump or pressure source) and reenter the initial fluid medium 102. In some examples, the flow rate supply is operable to move the transfected cell or cells to the holding chamber 110.

The system 120 may include a flow rate supply. In an example, the flow rate supply may be any device or instrument that provides a velocity to the cells in the system 120. The flow rate supply may provide an optimized cell velocity for a transfection event. In some examples, the flow rate supply may be configured to supply a cell velocity of about 1 mm/s to about 2 mm/s, about 2 mm/s to about 3 mm/s, about 3 mm/s to about 4 mm/s, about 4 mm/s to about 5 mm/s, about 5 mm/s to about 6 mm/s, about 6 mm/s to about 7 mm/s, about 7 mm/s to about 8 mm/s, about 8 mm/s to about 9 mm/s, about 9 mm/s to about 10 mm/s, about 10 mm/s to about 20 mm/s, about 20 mm/s to about 30 mm/s, about 30 mm/s to about 40 mm/s, about 40 mm/s to about 50 mm/s, about 50 mm/s to about 60 mm/s, about 60 mm/s to about 70 mm/s, about 70 mm/s to about 80 mm/s, about 80 mm/s to about 90 mm/s, about 90 mm/s to about 100 mm/s, about 100 mm/s to about 200 mm/s, about 200 mm/s to about 300 mm/s, about 300 mm/s to about 400 mm/s, or about 400 mm/s to about 500 mm/s, about 500 mm/s to about 750 mm/s, about 750 mm/s to about 1000 mm/s, about 1000 mm/s to about 2000 mm/s, about 2000 mm/s to about 5000 mm/s, or about 5000 mm/s to about 10000 mm/s. The velocity may be optimized depending on the characteristics of the cell or cells to be transfected.

In one example, the flow rate supply may be a pressure supply to apply a pressure (e.g., driving fluid pressure) of about 5 psi to about 100 psi to the system to move the cells between components. In an example, the pressure supply may supply a pressure of about 5 psi, about 10 psi, about 15 psi, about 20 psi, about 25 psi, about 30 psi, about 35 psi, about 40 psi, about 45 psi, about 50 psi, about 55 psi, about 60 psi, about 65 psi, about 70 psi, about 75 psi, about 80 psi, about 85 psi, about 90 psi, about 95 psi, or about 100 psi. In another example, the pressure supply may supply a pressure of about 5 psi to about 10 psi, about 10 psi to about 15 psi, about 15 psi to about 20 psi, about 20 psi to about 25 psi, about 25 psi to about 30 psi, about 30 psi to about 35 psi, about 35 psi to about 40 psi, about 40 psi to about 45 psi, about 45 psi to about 50 psi, about 50 psi to about 55 psi, about 55 psi to about 60 psi, about 60 psi to about 65 psi, about 65 psi to about 70 psi, about 70 psi to about 75 psi, about 75 psi to about 80 psi, about 80 psi to about 85 psi, about 85 psi to about 90 psi, about 90 psi to about 95 psi, about 95 psi to about 100 psi. In an example, the pressure may be optimized depending on the characteristics of the cell or cells to be transfected. In some examples, the flow rate supply may supply a different flow rate for different movements of the cell or cells depending on a desired velocity. It will be appreciated that the flow rate supply described in this paragraph may be used in any of the other systems and methods described herein.

The pressure supply may comprise a regulator having one or multiple control systems. In an example, the regulator may provide pressure at locations where the cells need to move. For example, the regulator can selectively provide pressure from the pressure supply to the initial fluid medium 102, the first reservoir 104, the first pay load reservoir 106, and the first microfluidic transfection device 108. The regulator may supply pressure at a first location (e.g., the first reservoir 104) to move the cell or cells from the first reservoir 104 to the initial fluid medium 102 and through the first microfluidic transfection device 108 to the first holding chamber 110.

In another aspect, the system 120 may include multiple pressure supplies. Each pressure supply may have a regulator. In an example, a first pressure supply may have a regulator that may supply a pressure at a first location (e.g., the first reservoir 104) to move the cell or cells from the first reservoir 104 to the initial fluid medium 102 and through the first microfluidic transfection device 108 to the first holding chamber 110.

FIG. 1B illustrates a system 100 for high throughput introduction of a plurality of exogenous molecules into a cell. The system of FIG. 1B includes at least two of system 120 of FIG. 1A connected in series (e.g., sequentially). The system 100 may be configured to allow for multiple exogenous molecules to be transfected into a cell sequentially. The sequential nature of the system 100 may produce a transfected cell comprising multiple exogenous molecules in a short period of time. Two or more transfection events may be successfully completed in under an hour. In other examples, two or more transfection events may be successfully completed in under 1 hour to under 2 hours, under two hours to under 3 hours, under 3 hours to under 4 hours, under 4 hours to under 5 hours, under 5 hours to under 6 hours, under 6 hours to under 7 hours, under 7 hours to under 8 hours, under 8 hours to under 9 hours, under 9 hours to under 10 hours, under 10 hours to under 11 hours, under 11 hours to under 12 hours, under 12 hours to under 13 hours, under 13 hours to under 14 hours, under 14 hours to under 15 hours, under 15 hours to under 16 hours, under 16 hours to under 17 hours, under 17 hours to under 18 hours, under 18 hours to under 19 hours, under 19 hours to under 20 hours, under 20 hours to under 21 hours, under 21 hours to under 22 hours, under 22 hours to under 23 hours, under 23 hours to under 24 hours, under 48 hours, or under 72 hours.

As illustrated in FIG. 1B, the system 100 may include an initial fluid medium 102, a first reservoir 104, a first payload reservoir 106, a first microfluidic transfection device 108, a first holding chamber 110, a second payload reservoir 112, a second microfluidic transfection device 114, a second holding chamber 116, and a third payload reservoir 118. The system 100 may be operable to transfect a plurality of cells with at least one exogenous molecule. The first transfection device 108 and the second transfection device 114 may be arranged in series. The system 100, as illustrated in FIG. 1B, is configured to deliver a first exogenous molecule and a second exogenous molecule to a cell or plurality of cells. In some examples, the system 100 can deliver more than two exogenous molecules to a cell or plurality of cells. In some examples, the tubing 122 may contain the initial fluid medium 102. In other examples, the initial fluid medium 102 may be in the first reservoir 104 and transferred into the tubing 122 during operation. The first reservoir 104, first payload reservoir 106, first microfluidic transfection device 108, first holding chamber 110, second payload reservoir 112, second microfluidic transfection device 114, second holding chamber 116, and third payload reservoir 118 may be in fluid communication with the tubing 122. All the components of system 100 may be in fluid communication with the tubing 122.

As illustrated in FIG. 1B, the first reservoir 104 may be in fluid communication with the initial fluid medium 102. The first reservoir 104 may contain a cell or a plurality of cells. In an example, the cells may be homogenous or heterogenous. The first reservoir 104 may be operable to provide the plurality of cells to the initial fluid medium 102. Cells may be moved from the first reservoir 104 to the initial fluid medium 102 by applying a flow rate using a flow rate supply (e.g., pump or pressure supply) to provide a cell velocity to the cells.

As illustrated in FIG. 1B, the first payload reservoir 106 may be in fluid communication with the initial fluid medium 102. In some examples, the first payload reservoir 106 may be downstream of the first reservoir 104. The first payload reservoir 106 may contain a first exogenous molecule to be transfected into a cell. The first payload reservoir 106 may inject the exogenous molecule into the initial fluid medium 102 by using a flow rate supply (e.g., pump_. When the first exogenous molecule is delivered to the initial fluid medium 102, the first exogenous molecule and the cells may form a first composition.

The first exogenous molecule may comprise gene editing materials, nanoparticles, proteins, antigens, amino-acids, viruses or viral components, DNA and related material, RNA and related material, lipids and related materials, small molecules, and salts. In an example, the first exogenous molecule may comprise a nucleotide sequence encoding an amino acid sequence of interest. It will be appreciated that the types of exogenous molecules may be used in all of the systems and methods described herein. In some examples, the first exogenous molecule may be sgRNA and the second exogenous molecule may be CRISPR associated protein 9 (Cas9). In some examples, the first or second exogenous molecule may be a lyophilized gRNA. In other examples, the first exogenous molecule may be Cas9. In some examples, the first exogenous molecule may include two or more exogenous molecules comprising a vector. The types of exogenous molecules described in this paragraph may be used as a first, second, third, fourth, fifth, or Nth exogenous molecule to be transfected into a cell or a plurality of cells using any of the systems or methods described herein.

In some aspects, the first exogenous molecule may be used for a knock-out gene editing therapy. In an example, the second exogenous molecule may be used for a knock-in gene therapy. In other examples, either exogenous molecule may be used for a knock-in or a knock-out gene editing therapy. In some examples, the first exogenous molecule and the second exogenous molecule may be used in combination to perform a gene editing therapy. In some examples, the first exogenous molecule may be a plasmid configured to insert an attP sequence in a desired locus of a cell. The second exogenous molecule may be a plasmid configured to perform an attB docking sequence. For example, a plasmid may comprise units to express the nCas9-M-MLV fusion protein along with expression modules of pegRNA and gRNA to perform a CRISPR prime editing for inserting an attP site in the desired locus. A second plasmid may comprise PhiC31 (serine integrase). A third plasmid may be an insertion cassette containing CAR construct and an attB docking sequence.

In another aspect, the first exogenous molecule and the second exogenous molecule may be configured to produce a gene edit on a cell. In some examples, the first exogenous molecule may prepare the cell or cells for insertion of the second exogenous molecule. The second exogenous molecule may provide the gene edit (e.g., knock-in) and the first exogenous molecule may make space for the gene edit (e.g., knock-out).

As illustrated in FIG. 1B, the first composition may be passed through a first microfluidic transfection device 108. The first microfluidic transfection device 108 may be downstream of the first payload reservoir 106. The first microfluidic transfection device 108 may include an inlet configured to receive the first composition, a transfection component configured to transfect the cell or cells with the first exogenous molecule, thereby producing a first population of transfected cells or a first transfected cell, and an outlet configured to return the first population of transfected cells to the initial fluid medium. In an example, the first population of transfected cells or first transfected cell may comprise the first exogenous molecule.

The cell or cells may have a successful transfection percentage of about 20% to about 25%, about 25% to about 30%, about 30% to about 35%, or about 35% to about 40% after being transfected using the first transfection device. In an example, the cell or cells may have a successful transfection percentage of about 20% to about 40%. In some examples, the transfection success rate in the optimized first transfection device may be about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the first transfection device 108. In an example, about 50% to about 90% of the cells may remain viable after being transfected using the first transfection device 108.

The transfection components of the microfluidic transfection devices disclosed herein may have various transfection parameters optimized for transfecting the cell or cells (e.g., to produce a high viability rate and a high successful transfection rate). In an example, the transfection parameters for optimized transfection of the cell or cells with an exogenous molecule may be device gap, supply pressure, supply flow rate, cell velocity, ridge number, channel width, channel height, channel length, gap width, gap size (e.g., height), gap length, ridge spacing, ridge angle, temperature, processing buffer constituents, cell type, cell source, and number of parallelized channels. Optimization, types, and functions of microfluidic transfection devices are described in U.S. Publication No. 2021/0292700A1, which is incorporated herein by reference in its entirety. It will be appreciated that the optimization or tuning of the microfluidic transfection device described in this paragraph may be applied to any of the microfluidic transfection devices in this system or in any other system or method described herein. Each microfluidic transfection device described in this system, or any other system or method described herein, may have optimized or tuned transfection parameters for a transfection event depending on the size, stiffness, adhesiveness, FACS characteristics, other physical characteristics (e.g., color, shape, etc.), or necessary or desired compression of the cell or cells to be transfected.

FIGS. 3A-B and 20 illustrate an example microfluidic transfection device 300 (e.g., first microfluidic transfection device 108 and/or second microfluidic transfection device 114). The microfluidic transfection device may have one or more inlets 302 and one or more outlets 304. The arrow 306 illustrates the direction of the flow of cells 310, exogenous molecules 308, and/or compositions through the microfluidic transfection device 300. The microfluidic transfection device 300 may include a liquid media 312. The microfluidic transfection device 300 may include one or more ridges 314. For example, the microfluidic transfection device 300 may have one ridge as illustrated in FIG. 3A. In an example, the microfluidic transfection device 300 may have two ridges as illustrated in FIG. 3B. The microfluidic transfection device 300 may have a first wall 316 (e.g., top wall) having a first interior surface 326 and a second wall 318 (e.g., bottom wall) having a second interior surface 328. The microfluidic transfection device 300 may have a third wall 336 (e.g., first side wall) and a fourth wall 338 (e.g., second side wall). The microfluidic transfection device 300 may have a recovery space 320. The one or more ridges 314 may have a ridge spacing 322. The one or more ridges 314 may have a ridge surface 332. The microfluidic transfection device 300 may have a gap 324. The microfluidic transfection device 300 may have cell counters 334. After passing through the components of the microfluidic transfection device 300, a transfected cell 330 may be produced.

In some examples, the microfluidic transfection device 300 may be made of elastomers such as polydimethylsiloxane (PDMS). In some examples, the microfluidic transfection device 300 may be made of PDMS, silicon, glass, metal, resin, epoxy resin, and/or combinations thereof. In some examples, the microfluidic transfection device 300 may be made of any suitable material.

In an aspect, the first wall 316, second wall 318, third wall 336, and fourth wall 338 may enclose the microfluidic transfection device 300 to have an interior height (IH) and an interior width (IW), as illustrated, for example, in FIGS. 3B and 20.

The inlets 302 may be located at different locations and at different angles, as illustrated, for example, in FIG. 20. In some examples, the angle (φ1 and/or φ2) may be between 20 degrees and 80 degrees. In some examples, the angle (φ1 and/or φ2) may be about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, about 45 degrees to about 50 degrees, about 50 degrees to about 55 degrees, about 55 degrees to about 60 degrees, about 60 degrees to about 65 degrees, about 65 degrees to about 70 degrees, about 70 degrees to about 75 degrees, about 75 degrees to about 80 degrees, about 80 degrees to about 85 degrees, or about 85 degrees to about 90 degrees. In some examples, one or more inlets 302 may be located at a point after one of the one or more ridges 314, as illustrated, for example, in FIG. 20.

The gap 324 of the transfection device 300 (e.g., 108, 114) may be the distance between the surface 332 of the one or more ridges 314 and the second interior surface 328. The gap 324 may be smaller than the height of the cell or cells to be transfected. The transfection component of the microfluidic transfection device 300 may have a gap 324 of about 5.4 μm to about 5.6 μm. In a further example, the transfection component may have a gap 324 of about 0.5 μm to about 1 μm, about 1 μm to about 1.5 μm, about 1.5 μm to about 2 μm, about 2 μm to about 2.5 μm, about 2.5 μm to about 3 μm, about 3 μm to about 3.5 μm, about 4 μm to about 4.5 μm, about 4.5 μm to about 5 μm, about 5 μm to about 5.5 μm, about 5.5 μm to about 6 μm, about 6 μm to about 6.5 μm, about 6.5 μm to about 7 μm, about 7 μm to about 7.5 μm, about 7.5 μm to about 8 μm, about 8 μm to about 8.5 μm, about 8.5 μm to about 9 μm, about 9 μm to about 9.5 μm, or about 9.5 μm to about 10 μm. In another example, the gap 324 may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, or about 90 μm to about 100 μm. In an example, the gap 324 may be optimized based on the type and size of the cell, the compression needed or desired, and other characteristics of microfluidic transfection. By optimizing the gap 324 of the transfection component of the microfluidic transfection device 300, the viability percentage of cells after transfection and the successful transfection percentage of cells may be optimized. In an example, the gap size may be optimized by decreasing the gap size to a value lower than the size of the cell or cells to be transfected (e.g., smaller cells have smaller gap sizes and larger cells have larger gap sizes). In an example, the optimized gap 324 for a T-cell may be about 5.3 μm to about 5.6 μm.

As illustrated in FIGS. 3A-B and 20, cells may be transfected by flowing through the gap 324 along flow path A1. Abnormal cells (e.g., cells with low compressibility) may flow along flow path A2 and out of the microfluidic transfection device 300 without being transfected. By flowing abnormal cells out of the microfluidic transfection device 300 without allowing them to pass through the gap, clogging or other negative effects may be prevented.

The transfection component of the microfluidic transfection device 300 may have a gap width and gap length (e.g., length of the ridge surface 332) optimized for transfection of the cell or cells with the first exogenous molecule depending on the size, stiffness, FACS characteristics, other physical cell properties of the cell or cells or the compression necessary or desired for the transfection event. In an example, the optimal gap width and gap length may depend on the type of cell and the type of exogenous molecule. In an example, the gap width may be about 5 μm and the gap length may be about 5 μm. In some examples, the gap width may be about 1 μm to about 10 μm, about 10 μm to about 20 μm, about 20 μm to about 30 μm, about 30 μm to about 40 μm, about 40 μm to about 50 μm, about 50 μm to about 60 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, or about 700 μm to about 800 μm, or about 800 μm to about 1000 μm, or about 1000 μm to about 5000 μm, or about 5000 μm to about 10000 μm. In some examples, the gap length (e.g., ridge surface 332 length) may be about 1 μm to about 2 μm, about 2 μm to about 3 μm, about 3 μm to about 4 μm, about 4 μm to about 5 μm, about 5 μm to about 6 μm, about 6 μm to about 7 μm, about 7 μm to about 8 μm, about 8 μm to about 9 μm, about 9 μm to about 10 μm, about 10 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, or about 700 μm to about 800 μm. In an example, for a T-cell the optimal gap width may be 12 μm and the optimal gap length may be 12 μm.

The transfection component of the microfluidic transfection device 300 may have a channel cross-sectional dimension (e.g., diameter) and channel length optimized for transfection of the cell or cells with the first exogenous molecule depending on the size, stiffness, FACS characteristics, or other physical properties of the cell or cells. FIGS. 3A-3B illustrate a single channel microfluidic transfection device 300. In an example, the optimal channel cross-sectional dimension and channel length may depend on the type of cell. In an example, the channel cross-section dimension may be about 1 μm to about 10 μm, about 10 μm to about 20 μm, about 20 μm to about 30 μm, about 30 μm to about 40 μm, about 40 μm to about 50 μm, about 60 μm to about 70 μm, about 70 μm to about 80 μm, about 80 μm to about 90 μm, about 90 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700, about 700 μm to about 800 μm, about 800 μm to about 900 μm, about 900 μm to about 1000 μm, about 1000 to about 1250 μm, about 1250 μm to about 1500 μm, about 1500 μm to about 1750 μm, or about 1750 μm to about 2000 μm. The channel length may be about 1 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 2000 μm, about 2000 μm to about 3000 μm, about 3000 μm to about 4000 μm, about 4000 μm to about 5000 μm, or more.

The transfection component of the microfluidic transfection device 300 may have an optimal number of ridges to transfect the cell or cells with an exogenous molecule depending on the size, stiffness, FACS characteristics, or other physical properties of the cell or cells. In an example, the first transfection component may have about 1, 2, 3, 4, 5, 6, 7, 8, 9, 10, 11, 12, 13, 14, 15, 16, or more ridges. For example, T-cells may exhibit the same or better results for 1 ridge (e.g., as illustrated in FIG. 3A) as compared to 5. There are other processing metrics aside from cell metrics (i.e., viability and transfection) to using 1 ridge, including reduced running pressure and pressure requirements overall, higher throughput, etc. However, for other cell types different ridge numbers may perform better. For example, early testing of peripheral blood mononuclear cells shows generally better results with 5 ridges than 1 ridge.

In an aspect, the one or more ridges 314 may be rectangular and extend into the microfluidic transfection device 300 at a 90 degree angle from the first interior surface 326. As illustrated in FIG. 20, the one or more ridges 314 may extend into the microfluidic transfection device 300 at an angle α. In some examples, the angle α may be about a 10 degree angle, about a 20 degree angle, about a 30 degree angle, about a 40 degree angle, about a 50 degree angle, about a 60 degree angle, about a 70 degree angle, about an 80 degree angle, or about a 90 degree angle from the first interior surface 326. In other examples, the one or more ridges 314 may be rounded, pointed, or have any other shape. In some examples, the one or more ridges 314 may be trapezoidal, triangular, or ellipsoidal. In some examples, the one or more ridges 314 may have an angled upper surface. For example, the one or more ridges 314 may increase in dimension (e.g., height within the channel) from a front surface (e.g., proximal inlet 302) to a back surface (e.g., distal to inlet 302). In some examples, the one or more ridges 314 may have rounded edges. For example, the one or more ridges 314 may have rounded edges rather than the square edges shown in FIGS. 3A-3B. The rounded edges may have a radius of about 0.05 micrometers (μm) to about 0.1 μm, about 0.1 μm to about 0.5 μm, about 0.5 μm to about 1.0 μm, about 1.0 μm to about 1.5 μm, about 1.5 μm to about 2.0 μm, about 2.0 micrometers to about 2.5 μm, about 2.5 μm to about 3.0 μm, about 3.0 μm to about 3.5 μm, about 3.5 μm to about 4.0 μm, about 4.0 μm to about 4.5 μm, about 4.5 μm to about 5.0 μm, about 5.0 μm to about 5.5 μm, about 5.5 μm to about 6.0 μm, about 6.0 μm to about 6.5 μm, about 6.5 μm to about 7.0 μm, about 7.0 μm to about 7.5 μm, about 7.5 μm to about 8.0 μm, about 8.0 μm to about 8.5 μm, about 8.5 μm to about 9.0 μm, about 9.0 μm to about 9.5 μm, about 9.5 μm to about 10.0 μm, or more.

In an aspect, the one or more ridges 314 may have a surface roughness at the ridge surface 332. In some examples, the one or more ridges 314 may have a surface roughness of about 1 nm to about 10 nm, about 10 nm to about 20 nm, about 20 nm to about 30 nm, about 30 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 100 nm, about 100 nm to about 200 nm, about 200 nm to about 300 nm, about 300 nm to about 400 nm, about 400 nm to about 500 nm, about 500 nm to about 600 nm, about 600 nm to about 700 nm, about 700 nm to about 800 nm, about 800 nm to about 900 nm, about 900 nm to about 1000 nm, about 1000 nm to about 1500 nm, or about 1500 nm to about 2000 nm.

The transfection component of the microfluidic transfection device 300 may have optimal ridge spacing 322 for transfection of the cell or cells with an exogenous molecule depending on the size, stiffness, FACS characteristics, or other physical properties of the cell or cells. In an example, the optimal ridge spacing 322 may depend on the type of cell and the type of first exogenous molecule. In an example, the optimal ridge spacing may be about 100 μm. In some examples, the ridge spacing 322 may be about 10 μm to about 50 μm, about 50 μm to about 100 μm, about 100 μm to about 200 μm, about 200 μm to about 300 μm, about 300 μm to about 400 μm, about 400 μm to about 500 μm, about 500 μm to about 600 μm, about 600 μm to about 700 μm, about 700 μm to about 800 μm, about 800 μm to about 900 μm, about 900 μm to about 1000 μm, about 1000 μm to about 5000 μm, or about 5000 μm to about 50000 μm.

The system may include processing buffer constituents. In an example, the processing buffer constituents may be selected based on cell type (e.g., size, stiffness, FACS characteristics, and/or other physical characteristics of the cell or cells). The processing buffer constituents may be selected from buffer constituents configured for microfluidic transfection devices. For example, a basal cell culture medium may be the processing buffer. In other examples, buffer constituents may be chosen based on whether the cells are primary cells (i.e., from a patient) or from cell lines (i.e., immortal test cell types).

In another aspect, the microfluidic transfection device 300 may have multiple parallelized channels for transfecting the cell or cells with an exogenous molecule. In some examples, parallelized channels may mean that multiple channels are stacked on top of one another. For example, FIGS. 3A-3B show a single channel of the microfluidic transfection device 300. Multiple channels may be parallelized (e.g., stacked on top of each other and/or horizontally placed next to one another). In some examples, the microfluidic transfection device 300 may have about 2, 3, 4, 5, 6, 7, 8, 9, 10, 11, 12, 13, 14, 15, 16, 17, 18, 19, 20 parallelized channels. In other examples, the microfluidic transfection device 300 may have about 1 to about 10 parallelized channels, about 10 to about 100 parallelized channels, about 100 to about 500 parallelized channels, about 500 to about 1000 parallelized channels, about 1000 to about 5000 parallelized channels, about 5000 to about 15000 parallelized channels, or more parallelized channels.

In further aspects, the transfection parameters of the microfluidic transfection device 300 may affect each other. For example, increasing the number of ridges may increase the necessary pressure. In other examples, some transfection parameters may require other transfection parameters to be adjusted to optimize the microfluidic transfection device.

Referring back to FIG. 1B, the first microfluidic transfection device 108 may have optimized transfection parameters for transfecting the cell or cells with the first exogenous molecule.

After the cell or cells have been transfected by the first microfluidic transfection device 108 (e.g., first transfection event), the cell or cells are returned to the initial fluid medium 102 via the outlet in the first microfluidic transfection device 108. As illustrated in FIG. 1B, the cell or cells may flow through the initial fluid medium 102 to a first holding chamber 110. The cells in the first holding chamber 110 can be referred to as a first transfected cell or first transfected population of cells (e.g., transfected with the first exogenous molecule). The first holding chamber 110 may be downstream of the first transfection device 108.

As illustrated in FIG. 1B, the first holding chamber 110 may have an inlet in fluid communication with the initial fluid medium 102 and configured to receive the first population of transfected cells or first transfected cell, a payload inlet in fluid communication with a second payload reservoir 112 and configured to receive a second exogenous molecule into the first holding chamber 110, and an outlet in fluid communication with the initial fluid medium 102. In an example, the first population of transfected cells or first transfected cell may be optionally held in the holding chamber 110 for a sufficient period of time (e.g., a holding period) to allow the first population of transfected cells or first transfected cell to recover from the first transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period may be more than 60 minutes. For example, the holding period may be up to 24 hours. In some examples, the holding period can allow the cells sufficient time and conditions to express one or more nucleotide sequences. In another example, the cells may be immediately injected with a second payload when they reach the holding chamber and have no holding period (e.g., no intervening culture step). It will be appreciated that the holding period as described in this paragraph may be applied to any system or method described herein.

As illustrated in FIG. 1B, the second payload reservoir 112 may be in fluid communication with the payload inlet of the first holding chamber 110. The second payload reservoir 112 may provide a second exogenous molecule to the first population of transfected cells or first transfected cell in the first holding chamber 110. In an example, the second exogenous molecule and the first population of transfected cells or first transfected cell may be combined in the first holding chamber 110 to form a second composition. In one example, the second exogenous molecule may be a different exogenous molecule from the first exogenous molecule. In another example, the first exogenous molecule and the second exogenous molecule may be the same exogenous molecule.

A flow rate supply (e.g., pump or pressure supply) may move the second composition from the first holding chamber 110 into the initial fluid medium 102 by providing a cell velocity to the second composition. In an example, the second composition may move through the initial fluid medium 102 to the second microfluidic transfection device 114.

As illustrated in FIG. 1B, the second composition may be passed through a second microfluidic transfection device 114. The second microfluidic transfection device 114 may include an inlet configured to receive the second composition, a transfection component configured to transfect the second composition with the second exogenous molecule, thereby producing a second population of transfected cells or a second transfected cell, and an outlet configured to return the second population of transfected cells or a second transfected cell to the initial fluid medium. In an example, the second population of transfected cells or second transfected cell may comprise the first exogenous molecule and the second exogenous molecule.

The cell or cells may have a successful transfection percentage of about 20% to about 25%, about 25% to about 30%, about 30% to about 35%, about 35% to about 40%, or more than 40% after being transfected using the second transfection device 114. In an example, the cell or cells may have a successful transfection percentage of about 20% to about 40% using the second transfection device. In some examples, the transfection success rate in the optimized second transfection device may be about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more than 90% of the cells may remain viable after being transfected using the second transfection device. In an example, about 50% to about 90% of the cells may remain viable after being transfected using the second transfection device.

The transfection component of the second microfluidic transfection device 114 may have transfection parameters optimized or tuned for the second transfection event (e.g., transfecting the cell or cells in the second composition with the second exogenous molecule). In an example, the transfection component of the second microfluidic transfection device 114 may have the same optimized transfection parameters as the transfection component of the first microfluidic transfection device 108. In another example, the transfection component of the second transfection device may have different transfection parameters than the transfection component of the first microfluidic transfection device 108. In some examples, optimizing the first microfluidic transfection device 108 and the second microfluidic transfection device 114 may further include optimizing the pressure provided to the system 100 or other parameters of the system.

As illustrated in FIG. 1B, after the cell or cells have been transfected by the second microfluidic transfection device 114, the cells are returned to the initial fluid medium 102 via the outlet in the second microfluidic transfection device 114. As illustrated in FIG. 1B, the cells may flow through the initial fluid medium 102 to a second holding chamber 116 by providing a flow rate using a flow rate supply. The second holding chamber 116 may be downstream of the second microfluidic transfection device 114.

As illustrated in FIG. 1B, the second holding chamber 116 may have an inlet in fluid communication with the initial fluid medium 102 configured to receive the second population of transfected cells or a second transfected cell, a payload inlet in fluid communication with a third payload reservoir 118 configured to receive a third exogenous molecule into the second holding chamber 116, and an outlet in fluid communication with the initial fluid medium 102. In an example, the second population of transfected cells or second transfected cell may be optionally held in the second holding chamber for a holding period. In another example, the cells may be immediately injected with a third payload when they reach the second holding chamber 116 and have no holding period (e.g., no intervening culture step).

The second holding chamber 116 may have an outlet configured to remove the cell or cells from the system 100 if only two transfection events are desired. In another example, the second population of cells or second transfected cell may undergo a third transfection event.

As illustrated in FIG. 1B, the third payload reservoir 118 may be in fluid communication with the payload inlet of the second holding chamber 116. The third payload reservoir 118 may provide a third exogenous molecule to the second population of transfected cells in the second holding chamber. In an example, the third exogenous molecule and the second population of transfected cells or second transfected cell may be combined in the second holding chamber to form a third composition. In one example, the third exogenous molecule may be a different exogenous molecule from the first exogenous molecule and/or second exogenous molecule. In another example, the first exogenous molecule, second exogenous molecule, and third exogenous molecule may be the same exogenous molecule. In a further example, the first exogenous molecule may be the same as the second exogenous molecule but different than the third exogenous molecule. In another example, the second exogenous molecule and the third exogenous molecule may be the same exogenous molecule, and the first exogenous molecule may be different from the second exogenous molecule and the third exogenous molecule.

A flow rate supply (e.g., pump or pressure supply) may move the third composition from the second holding chamber 116 into the initial fluid medium 102 by providing a cell velocity to the cell or cells. The third composition may move through the initial fluid medium to a third microfluidic transfection device (not shown). The third microfluidic transfection device may be operable to transfect the cells in the third composition with the third exogenous molecule (e.g., third transfection event). The third microfluidic transfection device may have transfection parameters tuned or optimized for the third transfection event.

The system may include a flow rate supply. In an example, the flow rate supply may be any device or instrument that provides a velocity to the cells in the system. The flow rate supply may provide an optimized cell velocity for a transfection event. In some examples, the flow rate supply may be configured to supply a cell velocity of about 1 mm/s to about 2 mm/s, about 2 mm/s to about 3 mm/s, about 3 mm/s to about 4 mm/s, about 4 mm/s to about 5 mm/s, about 5 mm/s to about 6 mm/s, about 6 mm/s to about 7 mm/s, about 7 mm/s to about 8 mm/s, about 8 mm/s to about 9 mm/s, about 9 mm/s to about 10 mm/s, about 10 mm/s to about 20 mm/s, about 20 mm/s to about 30 mm/s, about 30 mm/s to about 40 mm/s, about 40 mm/s to about 50 mm/s, about 50 mm/s to about 60 mm/s, about 60 mm/s to about 70 mm/s, about 70 mm/s to about 80 mm/s, about 80 mm/s to about 90 mm/s, about 90 mm/s to about 100 mm/s, about 100 mm/s to about 200 mm/s, about 200 mm/s to about 300 mm/s, about 300 mm/s to about 400 mm/s, or about 400 mm/s to about 500 mm/s, about 500 mm/s to about 750 mm/s, about 750 mm/s to about 1000 mm/s, about 1000 mm/s to about 2000 mm/s, about 2000 mm/s to about 5000 mm/s, or about 5000 mm/s to about 10000 mm/s. The velocity may be optimized depending on the characteristics of the cell or cells to be transfected.

In one example, the flow rate supply may be a pressure supply to apply a pressure (e.g., driving fluid pressure) of about 5 psi to about 100 psi to the system to move the cells between components. In an example, the pressure supply may supply a pressure of about 5 psi, about 10 psi, about 15 psi, about 20 psi, about 25 psi, about 30 psi, about 35 psi, about 40 psi, about 45 psi, about 50 psi, about 55 psi, about 60 psi, about 65 psi, about 70 psi, about 75 psi, about 80 psi, about 85 psi, about 90 psi, about 95 psi, or about 100 psi. In another example, the pressure supply may supply a pressure of about 5 psi to about 10 psi, about 10 psi to about 15 psi, about 15 psi to about 20 psi, about 20 psi to about 25 psi, about 25 psi to about 30 psi, about 30 psi to about 35 psi, about 35 psi to about 40 psi, about 40 psi to about 45 psi, about 45 psi to about 50 psi, about 50 psi to about 55 psi, about 55 psi to about 60 psi, about 60 psi to about 65 psi, about 65 psi to about 70 psi, about 70 psi to about 75 psi, about 75 psi to about 80 psi, about 80 psi to about 85 psi, about 85 psi to about 90 psi, about 90 psi to about 95 psi, about 95 psi to about 100 psi. In an example, the pressure may be optimized depending on the characteristics of the cell or cells to be transfected. In some examples, the flow rate supply may supply a different flow rate for different movements of the cell or cells depending on a desired velocity. It will be appreciated that the flow rate supply described in this paragraph may be used in any of the other systems and methods described herein.

The pressure supply may include a regulator having one or multiple control systems. In an example, the regulator may provide pressure at locations where the cells need to move. For example, the regulator may be operable to provide directed pressure to the first reservoir 104 to direct the cells to the initial fluid medium, directed pressure to the first payload reservoir 106 to move the first exogenous molecule to the initial fluid medium 102, directed pressure to the initial fluid medium to move the cells and first exogenous molecule through the first microfluidic transfection device 108, directed pressure to the first microfluidic transfection device to move the transfected cell or transfected cells to the first holding chamber 110, directed pressure to move the second exogenous molecule from the second payload reservoir 112 to the first holding chamber 110, directed pressure to the first holding chamber 110 to move the second composition (e.g., transfected cell or cells and second exogenous molecule) to the second microfluidic transfection device 114, directed pressure to the second microfluidic transfection device 114 to move the second transfected cell or cells (e.g., the cells comprising the first and second exogenous molecule) to the second holding chamber 116, directed pressure to the third payload reservoir 118 to move the third exogenous molecule to the second holding chamber, and so on. The regulator may supply pressure at a first location (e.g., the first cell reservoir 104) to move the cell or cells from the first reservoir 104 to the initial fluid medium 102 and through the first microfluidic transfection device 108 to the first holding chamber 110. The regulator may supply a pressure at a second location (e.g., the first holding chamber 110) to move the second composition of cells from the first holding chamber 110 to the initial fluid medium 102 and through the second microfluidic transfection device 114 to the second holding chamber. The regulator may supply a pressure at a third location (e.g., the second holding chamber) to move the third composition of cells from the second holding chamber 116 to the initial fluid medium 102 and through the third microfluidic transfection device.

In another aspect, the system 100 may include multiple pressure supplies. Each pressure supply may have a regulator. In an example, a first pressure supply may have a regulator that may supply a pressure at a first location (e.g., the first cell reservoir) to move the cell or cells from the first reservoir 104 to the initial fluid medium 102 and through the first microfluidic transfection device 108 to the first holding chamber 110. A second pressure supply may have a regulator that may supply a pressure at a second location (e.g., the first holding chamber) to move the second composition of cells from the first holding chamber 110 to the initial fluid medium 102 and through the second microfluidic transfection device 114 to the second holding chamber 116. A third pressure supply may supply a pressure at a third location (e.g., the second holding chamber 116) to move the third composition of cells from the second holding chamber 116 to the initial fluid medium 102 and through the third microfluidic transfection device.

The system may include N holding chambers, transfection devices, and payload reservoirs configured to transfect N exogenous molecules into the cell or cells. The N holding chambers, transfection devices, and payload reservoirs may have the same structures, characteristics and properties as the first and second holding chambers, transfection devices, and payload reservoirs. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Further provided herein is a method 200 for high throughput introduction of a plurality of exogenous molecules into a cell. The method 200 may produce transfected cells comprising one or more exogenous molecules at a rapid pace (e.g., multiple transfection events can occur in less than 24 hours, and, in some examples, less than 1 hour). A first transfection event and a second transfection event may be completed in under an hour. The transfected cell or cells may be transfected with two or more exogenous molecules sequentially and produce a high transfection rate (e.g., expression percentage) and high viability percentage compared to methods known in the art. Expression percentage means a percentage of the cells that express a particular gene or gene as intended by the transfection of an exogenous molecule into the cell. The method 200 may be completed in a fraction of the time of methods known in the art. The method 200 may be conducted using any of the systems described herein. In some examples, the method 200 is conducted using system 100 and/or system 120.

In some examples, sequential transfection events (e.g., a first transfection of cells with a first exogenous molecule and a second transfection of cells with a second exogenous molecule) may be completed in under about 24 hours. In some examples, sequential transfection events may be completed in under about 10 minutes to under about 30 minutes, under about 30 minutes to under about 1 hour, under about 1 hour to under about 2 hours, under about 2 hours to under about 3 hours, under about 3 hours to under about 4 hours, under about 4 hours to under about 5 hours, under about 5 hours to under about 6 hours, under about 6 hours to under about 7 hours, under about 7 hours to under about 8 hours, under about 8 hours to under about 9 hours, under about 9 hours to under about 10 hours, under about 10 hours to under about 11 hours, under about 11 hours to under about 12 hours, under about 12 hours to under about 13 hours, under about 13 hours to under about 14 hours, under about 14 hours to under about 15 hours, under about 15 hours to under about 16 hours, under about 16 hours to under about 17 hours, under about 17 hours to under about 18 hours, under about 18 hours to under about 19 hours, under about 19 hours to under about 20 hours, under about 20 hours to under about 21 hours, under about 21 hours to under about 22 hours, under about 22 hours to under about 23 hours, or under about 23 hours to under about 24 hours.

In some examples, sequential delivery (e.g., a first transfection event for a first exogenous molecule and a second transfection even for a second exogenous molecule) may result in a greater cell viability and greater transfection success rate (e.g., expression percentage) than co-delivery (e.g., delivery of the first exogenous molecule and the second exogenous molecule at the same time). In some examples, sequential delivery can result in higher viability and higher transfection success rate than a single transfection with a long holding period followed by a second transfection.

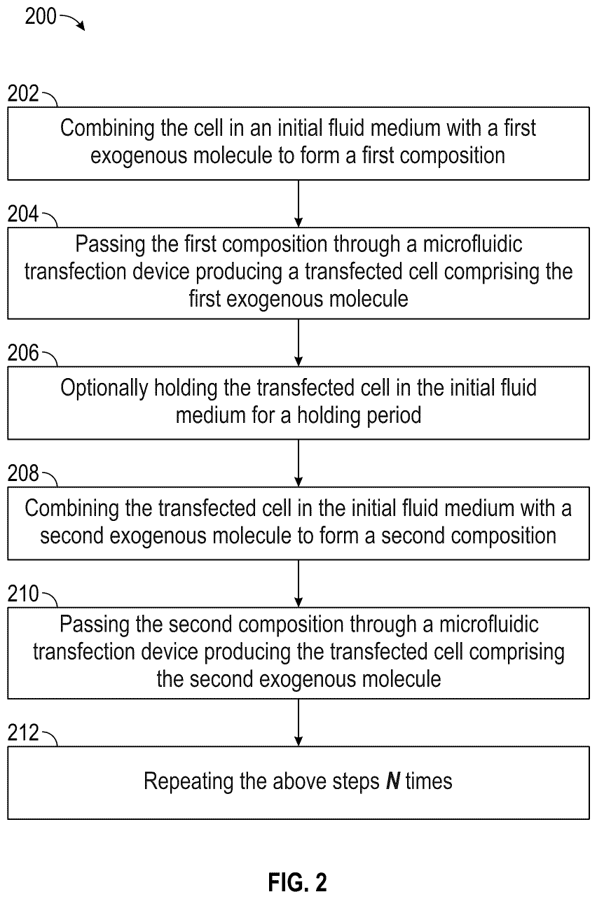

As illustrated in FIG. 2, the method 200 may comprise one or more steps. At block 202, the method 200 includes combining the cell in an initial fluid medium with a first exogenous molecule to form a first composition. The first exogenous molecule may be any of the exogenous molecules described herein. The first exogenous molecule may be held in a payload reservoir and provided to the initial fluid medium by a flow rate supply as described herein.

As illustrated in FIG. 2, the second step 204 may comprise passing the first composition through a microfluidic transfection device producing a transfected cell comprising the first exogenous molecule. In an example, the transfection device may have various transfection parameters for an optimal first transfection event. The first composition may be passed through the microfluidic transfection device by providing a flow rate to the initial fluid medium and thereby to the first composition via a flow rate supply as described herein.

The transfection parameters may optimize the first transfection event. The first transfection event may result in optimal cell viability percentages and optimal transfection success percentages. In an example, the optimized first transfection event results in a cell viability percentage of about 50% to about 55%, about 55% to about 60%, about 60% to about 65%, about 65% to about 70%, about 70% to about 75%, about 75% to about 80%, about 80% to about 85%, about 85% to about 90%, or more. In a further example, the transfection success rate (e.g., expression percentage) in the optimized first transfection device is about 20% to about 40%. In some examples, the transfection success rate in the optimized first transfection device may be about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more.

As illustrated in FIG. 2, at step 206, the method 200 may include optionally holding the transfected cell in the initial fluid medium for a holding period. In some examples, the transfected cell can be held in a holding chamber for a holding period. The holding period may be any of the holding periods described herein. In an example, the transfected cell may be optionally held a sufficient period of time to allow the first population of transfected cells to recover from the first transfection event. In another example, the cells may be immediately injected with a second payload and have no holding period (e.g., no intervening culture step). The cells can be injected with the second payload in the initial fluid medium or in the holding chamber.

As illustrated in FIG. 2, at step 208, the method 200 may include combining the transfected cell in the initial fluid medium with a second exogenous molecule to form a second composition. In some examples, the second exogenous molecule may be a different exogenous molecule than the first exogenous molecule. In another example, the second exogenous molecule may be the same exogenous molecule as the first exogenous molecule. In some examples, the method 200 can include combining the transfected cell in the holding chamber with a second exogenous molecule instead of in the initial fluid medium.

As illustrated in FIG. 2, at step 210, the method 200 may include passing the second composition through a microfluidic transfection device producing the transfected cell comprising the second exogenous molecule (e.g., second transfection event). In an example, the transfection device may have various transfection parameters for an optimal second transfection event. The second composition can be providing a flow rate via the flow rate supply to pass the second composition through the microfluidic transfection device.

The second transfection event may result in optimal cell viability percentages and optimal transfection success percentages. In an example, the optimized second transfection event results in a cell viability percentage of about 50% to about 55%, about 55% to about 60%, about 60% to about 65%, about 65% to about 70%, about 70% to about 75%, about 75% to about 80%, about 80% to about 85%, about 85% to about 90%, or more. In a further example, the transfection success rate in the optimized second transfection device is about 20% to about 40%. In some examples, the transfection success rate in the optimized second transfection device may be about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. In some examples, the transfection success rate for the method 200 is higher than transfection success rates for other types of transfection methods because of the sequential delivery of the first exogenous molecule and the second exogenous molecule.

As illustrated in FIG. 2, at step 212, the method may include repeating steps 202-210 N times to transfect the cell with N exogenous molecules producing N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the exogenous molecule may be the same in some or all transfection events. In another example, the exogenous molecule may be different in all of the transfection events.

Also presented herein is a method for high throughput introduction of a plurality of exogenous molecules into a cell (or a plurality of cells). The method may include passing the cell through N microfluidic processing cycles wherein N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Each cycle may include combining the cell in an initial fluid medium with an exogenous molecule to form a composition and passing the composition through a microfluidic transfection device, thereby introducing the exogenous molecule to the cell to form a transfected cell comprising the exogenous molecule. In an example, the exogenous molecule may comprise the same or a different molecule for each cycle. In another example, some or all of the cycles may include the same exogenous molecule. In a further example, all of the cycles may include a different exogenous molecule.

The transfection parameters of the transfection device may be optimized for each transfection event. In some examples, the transfection parameters may be optimized based on the size of the cell, the cell membrane stiffness, or the cell's FACS characteristics.

There may be a holding period after each complete cycle. The transfected cell may be optionally held in a holding chamber for a sufficient period of time (e.g., a holding period) to allow the first population of transfected cells to recover from the first transfection event. In some examples, the holding period can allow the cells sufficient time and conditions to express one or more nucleotide sequences. In another example, the cells may be immediately injected with a second payload when the cells reach the holding chamber and have no holding period (e.g., no intervening culture step).

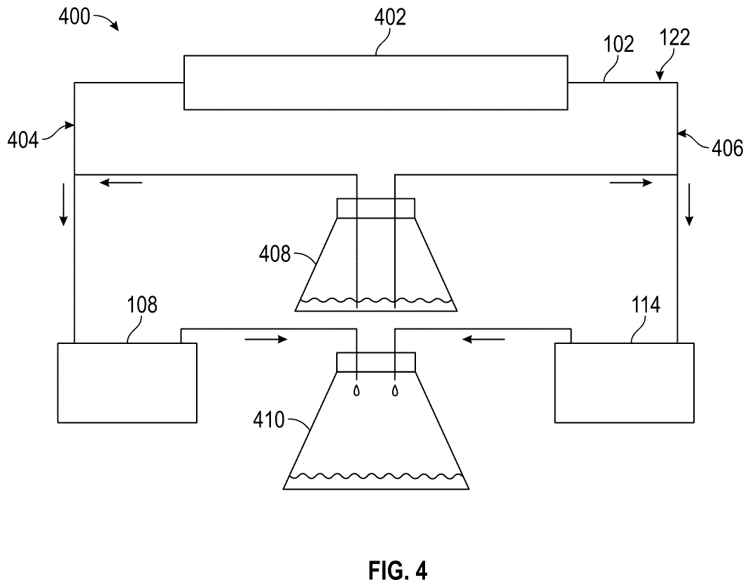

Also described herein is a system 400 for high throughput introduction of an exogenous molecule into cells in a population of cells which are heterogenous. The system 400 of FIG. 4 may be configured to transfect a heterogenous population of cells. The heterogenous cells may be sorted by a cell sorter into two or more groups (e.g., sub-populations). The groups may have different flow paths through different microfluidic transfection devices. The different microfluidic transfection devices may have different optimized transfection parameters depending on the group they are transfecting. The transfection parameters of each transfection device may be optimized based on the characteristics (e.g., size, stiffness, FACS characteristics, and/or other physical characteristics) of the group (i.e., sub-population) they are transfecting. The system 400 may provide rapid transfection of two or more groups of cells at optimal viability and successful transfection rates. The different microfluidic transfection device may be arranged in parallel such that the first transfection event and the second transfection event occur at substantially the same time, transfecting both groups of cells with an exogenous molecule rapidly.

As illustrated in FIG. 4, the system 400 may include an initial fluid medium 102, a cell sorter 402, a first payload reservoir 106, a first microfluidic transfection device 108, and a second microfluidic transfection device 114. The system 400 may be operable to transfect a plurality of cells with at least one exogenous molecule. In an example, the first transfection device and the second transfection device may be arranged in parallel. In some examples, the tubing 122 may contain the initial fluid medium 102. In other examples, the initial fluid medium 102 may be in the cell sorter 402 and transferred into the tubing 122 during operation. The cell sorter 402, first payload reservoir 106, the first microfluidic transfection device 108, and the second microfluidic transfection device 114 may be in fluid communication with the tubing 122. All the components of system 400 may be in fluid communication with the tubing 122.

As illustrated in FIG. 4, the cell sorter 402 may be in fluid communication with the initial fluid medium 102. The cell sorter 402 may be configured to sort a heterogenous population of cells (i.e., plurality of cells). In an example, the cell sorter may be configured to sort the plurality of cells into a first sub-population of cells and a second sub-population of cells. The cell sorter 402 may sort the plurality of cells based on size, cell stiffness, cell adhesiveness, FACS characteristics, and other physical properties of the cells (e.g., shape, color, hardness, malleability, solubility, density, etc.). In an example, the FACS characteristics may include phenotypes, receptor types, and frequencies. Cell frequency refers to the resonant frequency of the cells. It will be appreciated that the cell sorter 402 may be used in any other system or method described herein. In some examples, the cell sorter 402 may be upstream of the initial fluid medium.

The first sub-population of cells may have a cell diameter below a first diameter cut-off value. The second sub-population of cells may have a cell diameter above a first diameter cut-off value. In another example, the first sub-population may have a cell stiffness below a stiffness cut-off value and the second sub-population may have a cell stiffness above a stiffness cut-off value. In a further example, the first sub-population may have a FACS characteristic, and the second sub-population of cells may have another FACS characteristic. In a further example, the cell sorter may be configured to sort the cells into a three, four, five, six, seven, eight, nine, or ten sub-populations. It will be appreciated that the sorting characteristics described in this paragraph may be used in any other system or method described herein.

As illustrated in FIG. 4, the initial fluid medium 102 may include a first flow path 404 and a second flow path 406. In an example, the first flow path 404 and the second flow path 406 may be arranged in parallel (e.g., operable to contain materials concurrently). The first flow path 404 may be operable to deliver the first sub-population of cells to the first microfluidic transfection device 108 from the cell sorter 402. The second flow path 406 may be operable to deliver the second sub-population of cells to the second microfluidic transfection device 114 from the cell sorter 402.

As illustrated in FIG. 4, the first payload reservoir 106 may be in fluid communication with the initial fluid medium 102 (i.e., the first flow path 404 and the second flow path 406). The first payload reservoir 106 may be operable to inject an exogenous molecule into the initial fluid medium 102 (i.e., the first flow path 404 and the second flow path 406). In an example, the first payload reservoir 106 may inject the first sub-population of cells with the exogenous molecule in the first flow path 404 forming a first composition. The first payload reservoir 106 may inject the second sub-population of cells with the exogenous molecule in the second flow path 406 forming a second composition. The first payload reservoir 106 may be downstream of the cell sorter 402.

As illustrated in FIG. 4, the first flow path 404 may be in fluid communication with the first microfluidic transfection device 108. The first composition may be passed through the first microfluidic transfection device 108. The first microfluidic transfection device 108 may include an inlet configured to receive the first composition from the first flow path 404, a transfection component configured to transfect the first composition with the exogenous molecule, thereby producing a first population of transfected cells (e.g., first transfection event), and an outlet configured to return the first population of transfected cells to the initial fluid medium 102. The first microfluidic transfection device 108 may be downstream of the first payload reservoir 106.

The first sub-population of cells may have a successful transfection percentage of about 20% to about 25%, about 25% to about 30%, about 30% to about 35%, about 35% to about 40%, or more after being transfected using the first microfluidic transfection device 108. In an example, the first sub-population of cells may have a successful transfection percentage of about 20% to about 40%. In some examples, the first sub-population of cells may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the first microfluidic transfection device 108. In an example, about 50% to about 90% of the cells may remain viable after being transfected using the first microfluidic transfection device 108.

As illustrated in FIG. 4, the second flow path 406 may be in fluid communication with the second microfluidic transfection device 114. The second composition may be passed through the second microfluidic transfection device 114. The second microfluidic transfection device 114 may include an inlet configured to receive the second composition from the second flow path 406, a transfection component configured to transfect the second composition with the exogenous molecule, thereby producing a second population of transfected cells (e.g., second transfection event), and an outlet configured to return the second population of transfected cells to the initial fluid medium 102. The second microfluidic transfection device 114 may be downstream of the first payload reservoir 106.

The second sub-population of cells may have a successful transfection percentage of about 20% to about 25%, about 25% to about 30%, about 30% to about 35%, about 35% to about 40%, or more after being transfected using the second microfluidic transfection device 114. In an example, the second sub-population of cells may have a successful transfection percentage of about 20% to about 40%. In some examples, the second sub-population of cells may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the second microfluidic transfection device 114. In an example, about 50% to about 90% of the cells may remain viable after being transfected using the second microfluidic transfection device.

The transfection components of the first microfluidic transfection device 108 and the second microfluidic transfection device 114 may have various transfection parameters optimized for transfecting the first sub-population of cells and second sub-population of cells, respectively. In some examples, the transfection parameters of the first microfluidic transfection device 108 may be different from the transfection parameters of the second microfluidic transfection device 114, due to the differences in the first sub-population of cells and the second sub-population of cells. In an example, the transfection parameters of the first microfluidic transfection device 108 and second microfluidic transfection device 114 may be the same. In one example, the transfection parameters of the first microfluidic transfection device 108 may be optimized for smaller cells while the transfection parameters for the second microfluidic transfection device 114 may be optimized for larger cells. Similar optimizations of the transfection parameters may occur based on how the cells are initially sorted (e.g., the first microfluidic transfection device 108 being optimized for stiffer cells while the second microfluidic transfection device 114 is optimized for less stiff cells, etc.).

In one aspect, the first transfection event and the second transfection may occur at substantially the same time (e.g., concurrently). In another aspect, the first transfection event and the second transfection event may occur at different times.

As illustrated in FIG. 4, the system 400 may have a holding chamber 110 in fluid communication with the initial fluid medium 102 (i.e., the first flow path 404 and the second flow path 406). The holding chamber 110 may be downstream of the first microfluidic transfection device 108 and the second microfluidic transfection device 114. The holding chamber 110 may be operable to receive the first population of transfected cells and the second population of transfected cells. In an example, the first and second populations of transfected cells may be optionally held in the holding chamber 110 for a sufficient period of time (e.g., a holding period) to allow the first and second populations of transfected cells to recover from the first transfection event (e.g., the transfection of the first sub-population of cells and the second sub-population of cells with the exogenous molecule). In some examples, the holding period can allow the cells sufficient time and conditions to express one or more nucleotide sequences.

The system may include a flow rate supply (e.g., pump or pressure source). In an example, the flow rate supply may be any device or instrument that provides a velocity to the cells in the system. In one example, the flow rate supply may be a pressure supply to apply a pressure of about 5 psi to about 100 psi to the system 400 to move the cells through the system 400. The pressure supply may comprise a regulator having one or multiple control systems. In an example, the regulator may provide pressure at locations where the cells need to move. The regulator may supply pressure at a first location (e.g., the first flow path 404) to move the first sub-population of cells from the cell sorter 402 through the first microfluidic transfection device 108 device to the holding chamber 110. The regulator may supply a pressure at a second location (e.g., the second flow path 406) to move the second sub-population of cells from the cell sorter 402 through the second microfluidic transfection device 114 to the holding chamber 110.

In an aspect, the system 400 may include multiple pressure supplies. Each pressure supply may have a regulator. In an example, a first pressure supply may have a regulator that may supply a pressure at a first location (e.g., the first flow path 404) to move the first sub-population of cells from the cell sorter 402 through the first microfluidic transfection device 108 device to the holding chamber 110. A second pressure supply may have a regulator that may supply a pressure at a second location (e.g., the second flow path 406) to move the second sub-population of cells from the cell sorter 402 through the second microfluidic transfection device 114 to the holding chamber 110.

A third pressure supply may supply a pressure to the holding chamber 110 to move the first population of transfected cells and the second population of transfected cells back to the cell sorter 402. For example, the system 400 may include a return line allowing the first population of transfected cells and the second population of transfected cells to flow back to the cell sorter 402 directly from the holding chamber 110. The system 400 may be used to sort the cells and transfect the cells N times with N different exogenous molecules. In an example N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30. In an example, the cells are sorted between each new set of transfection events and the microfluidic transfection devices are tuned between each new set of transfection events. In some examples, each of the N transfection events may transfect a sub-population of cells with a different exogenous molecule. In other examples, each transfection event may comprise the same exogenous molecule. In further examples, some transfection events may comprise the same exogenous molecule and other transfection events may comprise different exogenous molecules.

In one aspect, the system of FIG. 1A and/or FIG. 1B may be connected with the system of FIG. 4 to transfect the cells using the system of FIG. 1A and/or FIG. 1B first and then transfect the cells with the system of FIG. 4. In another aspect, the system of FIG. 4 may transfect the cells first and then the system of FIG. 1B or FIG. 1A may transfect the cells.

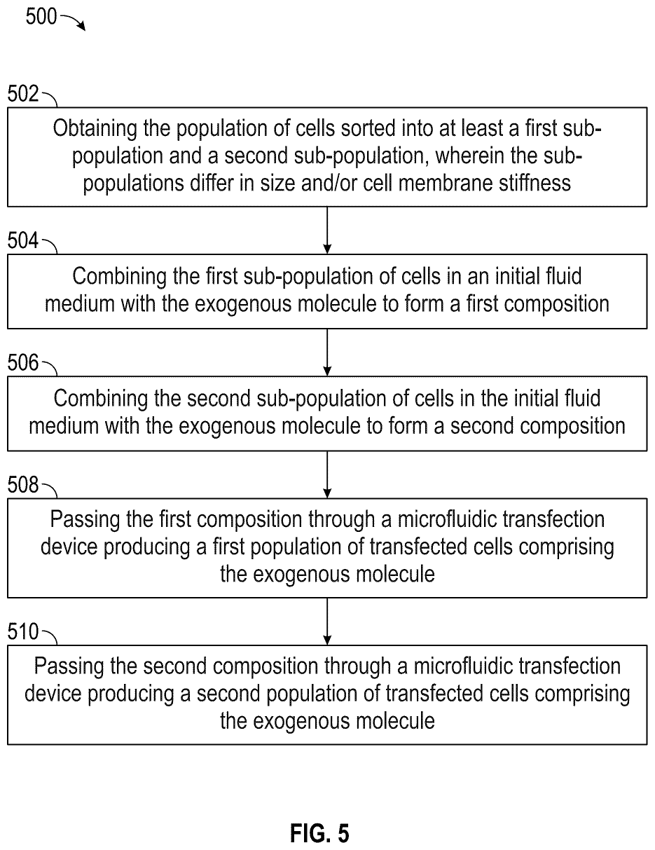

Further provided herein is a method for high throughput introduction of an exogenous molecule into cells in a population of cells which are heterogenous. The method of FIG. 5 may be configured to transfect a heterogenous population of cells. The heterogenous cells may be sorted by a cell sorter into two or more groups (e.g., sub-populations). The groups may have different flow paths through different microfluidic transfection devices. The different microfluidic transfection devices may have different optimized transfection parameters depending on the group they are transfecting. The transfection parameters of each transfection device may be optimized based on the characteristics (e.g., size, stiffness, FACS characteristics, and/or other physical properties of the cells as described herein) of the group (i.e., sub-population) they are transfecting. The method may provide rapid transfection of two or more groups of cells at optimal viability and successful transfection rates. The different microfluidic transfection devices may be arranged in parallel such that the first transfection event and the second transfection event occur at substantially the same time, transfecting both groups of cells with an exogenous molecule rapidly.

As illustrated in FIG. 5, the method 500 may comprise one or more steps. At a first step 502, the method 500 may include obtaining the population of cells sorted into at least a first sub-population and a second sub-population. The population of cells may be sorted by the cell sorter described herein. The first sub-population and the second sub-population may be differentiated by size, cell stiffness, cell adhesiveness, FACS characteristics, and/or other physical properties of the cells as provided herein. A flow rate may be provided to the first sub-population of cells to move the first sub-population cells through a first flow path of an initial fluid medium. A flow rate may be provided to the second sub-population of cells to the move the second sub-population of cells through a second flow path of an initial fluid medium. In an example, the first flow path may be in fluid communication with a first microfluidic transfection device. The second flow path may be in fluid communication with a second microfluidic transfection device.

As illustrated in FIG. 5, at a second step 504, the method 500 may include combining the first sub-population of cells in the initial fluid medium (e.g., first flow path) with the exogenous molecule to form a first composition. At a third step 506, the method 500 may include combining the second sub-population of cells in the initial fluid medium (e.g., second flow path) with the exogenous molecule to form a second composition. The exogenous molecule may be stored in a payload reservoir. The payload reservoir may provide the first sub-population of cells and the second sub-population of cells with the exogenous molecule to form the first composition and the second composition. The exogenous molecule may be any of the exogenous molecules described herein.

As illustrated in FIG. 5, at a fourth step 508, the method may include passing the first composition through a first microfluidic transfection device. In an example, the first microfluidic transfection device may have various transfection parameters (e.g., process parameters) optimized for transfecting the first sub-population of cells with the exogenous molecule (e.g., first composition). The first transfection event may result in optimal cell viability percentages and optimal transfection success percentages in the first sub-population of cells. In an example, the first transfection event results in a cell viability percentage of about 50% to about 55%, about 55% to about 60%, about 60% to about 65%, about 65% to about 70%, about 70% to about 75%, about 75% to about 80%, about 80% to about 85%, about 85% to about 90%, or more. In a further example, the transfection success rate in the optimized first transfection event is about 20% to about 40%. In some examples, the first sub-population of cells may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more.

As illustrated in FIG. 5, at a fifth step 510, the method may include passing the second composition through a second microfluidic transfection device, thereby producing a second population of transfected cells comprising the exogenous molecule. In an example, the second transfection device may have various transfection parameters (e.g., process parameters) optimized for transfecting the second sub-population of cells with the exogenous molecule (e.g., second composition). The transfection event may result in optimal cell viability percentages and optimal transfection success percentages in the second sub-population of cells. In an example, the optimized transfection event results in a cell viability percentage of about 50% to about 55%, about 55% to about 60%, about 60% to about 65%, about 65% to about 70%, about 70% to about 75%, about 75% to about 80%, about 80% to about 85%, about 85% to about 90%, or more. In a further example, the transfection success rate in the optimized second microfluidic transfection device is about 20% to about 40%. In some examples, the second sub-population of cells may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more.

In one aspect, the transfection of the first sub-population of cells and the transfection of the second sub-population of cells may occur substantially simultaneously (e.g., concurrently) in the first microfluidic transfection device and the second microfluidic transfection device.

The method 500 may include optionally holding the first population of transfected cells and the second population of transfected cells in a holding chamber for a holding period. In an example, the first population of transfected cells and the second population of transfected cells may be optionally held in the holding chamber for a sufficient period of time (e.g., a holding period) to allow the first population and second population of transfected cells to recover from the transfection events. In some examples, the holding period can allow the cells sufficient time and conditions to express one or more nucleotide sequences.

The method may include repeating steps 502-510 N times to transfect the cells with N exogenous molecules in N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, a different exogenous molecule may be transfected into each sub-population in each transfection event. In other examples, some or all of transfection events may transfect the same exogenous molecule into the first and second sub-populations of cells. In some examples, the first population of transfected cells and the second population of transfected cells experience a higher rate of successful transfection and viability then other known methods when transfected with a second exogenous molecule and N additional exogenous molecules sequentially (e.g., shortly after being transfected with the first exogenous molecule). For example, the first population of transfected cells and second population of transfected cells may be transfected with a second exogenous molecule within about 1 hour to about 2 hours, about 2 hours to about 6 hours, about 6 hours to about 12 hours, about 12 hours to about 18 hours, or within 24 hours of being transfected with the first exogenous molecule. The cells may exhibit a successful transfection rate (e.g., expression percentage) of about 20% to about 30%, about 30% to about 40%, about 40% to about 50%, about 50% to about 60%, about 60% to about 70%, about 70% to about 80%, or more after being transfected with the first exogenous molecule and the second exogenous molecule sequentially. In some examples, the cells may exhibit a viability of about 50% to about 60%, about 60% to about 70%, about 70% to about 80%, 80% to about 90%, or more after being transfected with the first exogenous molecule and the second exogenous molecule sequentially.

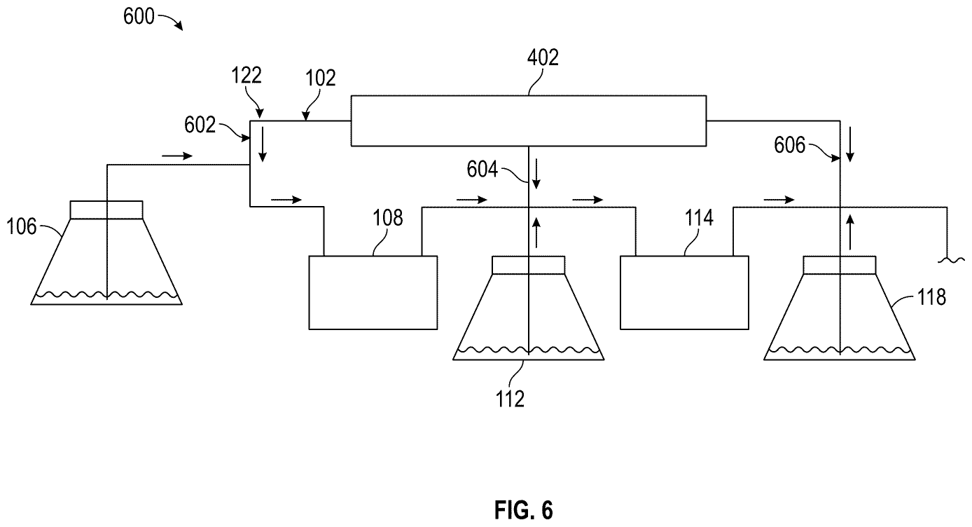

Also provided herein is a system 600 for transfecting a plurality of cells with a plurality of exogenous molecules. The system 600, as illustrated in FIG. 6, may be configured to sort the cells into at least two groups (i.e., sub-populations). The system 600 may be operable to conduct two or more transfection events sequentially. By transfecting the cells sequentially, groups of cells may be transfected quickly while retaining or improving on the successful transfection and viability rates of methods known in the art. The system 600 may be advantageous because it may be capable of successfully transfecting a heterogenous population of cells quickly without having to remove the cells from the system. In some examples, one sub-population may be transfected with two or more exogenous molecules, while another sub-population is only transfected with one exogenous molecule.

As illustrated in FIG. 6, the system 600 may include a cell sorter 402, an initial fluid medium 102, a first payload reservoir 106, a first microfluidic transfection device 108, a second payload reservoir 112, a second microfluidic transfection device 114, and a third payload reservoir 118. The system may be operable to transfect a plurality of cells with at least one exogenous molecule. The first microfluidic transfection device 108 and the second microfluidic transfection device 114 may be arranged in series. In some examples, the tubing 122 may contain the initial fluid medium 102. In other examples, the initial fluid medium 102 may be in the cell sorter 402 and transferred into the tubing 122 during operation. The cell sorter 402, first payload reservoir 106, first microfluidic transfection device 108, second payload reservoir 112, second microfluidic transfection device 114, and third payload reservoir 118 may be in fluid communication with the tubing 122. All the components of system 600 may be in fluid communication with the tubing 122.

As illustrated in FIG. 6, the cell sorter 402 may be in fluid communication with the initial fluid medium 102. The cell sorter 402 may be configured to sort a heterogenous population of cells (i.e., plurality of cells). In an example, the cell sorter 402 may be configured to sort the plurality of cells into a first sub-population of cells, a second sub-population of cells, and a third sub-population of cells. The cell sorter 402 may sort the plurality of cells based on size, cell membrane stiffness, FACS characteristics, and/or other physical characteristics of the cells. In an example, the FACS characteristics may include phenotypes, receptor types, and frequencies.

The first sub-population of cells may be moved through a first flow path 602 (e.g., from the cell sorter 402 to a position upstream of the first microfluidic transfection device 108) of the initial fluid medium 102 by supplying a flow rate using a flow rate supply to provide a cell velocity to the first sub-population of cells. The second sub-population of cells may be moved through a second flow path 604 (e.g., from the cell sorter 402 to a position downstream the first microfluidic transfection device 108 and in front of the second microfluidic transfection device 114) by supplying a flow rate using a flow rate supply. The third sub-population of cells may be moved through a third flow path 606 (e.g., from the cell sorter 402 to a position downstream the second microfluidic transfection device 114) of the initial fluid medium 102 by supplying a flow rate using a flow rate supply.

As illustrated in FIG. 6, the first payload reservoir 106 may be in fluid communication with the first flow path 602 of the initial fluid medium 102. In some examples, the first payload reservoir 106 may be downstream of the cell sorter 402 and upstream of the first microfluidic transfection device 108. The first payload reservoir 106 may contain a first exogenous molecule to be transfected into the first sub-population of cells. The first payload reservoir 106 may inject the exogenous molecule into the initial fluid medium 102 (e.g., the first flow path 602) by using a flow rate supply. When the first exogenous molecule is delivered to the initial fluid medium 102, the first exogenous molecule and the first sub-population of cells may form a first composition.

As illustrated in FIG. 6, the first composition may be passed through a first microfluidic transfection device 108. The first microfluidic transfection device 108 may include an inlet configured to receive the first composition, a transfection component configured to transfect the cell or cells with the first exogenous molecule, thereby producing a first population of transfected cells, and an outlet configured to return the first population of transfected cells to the initial fluid medium 102. In an example, the first population of transfected cells may comprise the first exogenous molecule. The transfection component of the first microfluidic transfection device 108 may have various transfection parameters optimized for transfecting the first sub-population of cells. The first sub-population of transfected cells may have a successful transfection percentage of about 20% to about 25%, about 25% to about 30%, about 30% to about 35%, about 35% to about 40%, or more after being transfected using the first microfluidic transfection device 108. In an example, the cell or cells may have a successful transfection percentage of about 20% to about 40%. In some examples, the first sub-population of cells may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the first microfluidic transfection device 108. In an example, about 50% to about 90% of the cells may remain viable after being transfected using the first microfluidic transfection device 108.

As illustrated in FIG. 6, after the first sub-population of cells have been transfected by the first microfluidic transfection device 108, the first population of transfected cells are returned to the initial fluid medium 102 via the outlet in the first microfluidic transfection device 108.

The first population of transfected cells may be optionally held in the initial fluid medium 102 for a sufficient period of time (e.g., a holding period) to allow the first population of transfected cells to recover from the first transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period can allow the cells sufficient time and conditions to express one or more nucleotide sequences. In another example, the first population of transfected cells may have no holding period.

As illustrated in FIG. 6, the second sub-population of cells may be combined with the first population of cells in the initial fluid medium 102, forming a heterogenous cell product. The second sub-population of cells may flow through the second flow path 604 of the initial fluid medium 102 to combine with the first population of transfected cells.

As illustrated in FIG. 6, the second payload reservoir 112 may be in fluid communication with the initial fluid medium 102 at a location upstream of the second microfluidic transfection device 114 and downstream of the first microfluidic transfection device 108. The second payload reservoir 112 may provide a second exogenous molecule to the first population of transfected cells and the second sub-population of cells. In an example, the second exogenous molecule, the first population of transfected cells, and the second sub-population of cells may be combined in the initial fluid medium to form a second composition. In one example, the second exogenous molecule may be a different exogenous molecule from the first exogenous molecule. In another example, the first exogenous molecule and the second exogenous molecule may be the same exogenous molecule. The second exogenous molecule may be any of the exogenous molecules described herein. A flow rate supply may move the second composition through the initial fluid medium 102 and through the second microfluidic transfection device 114 by providing a velocity to the cells.

As illustrated in FIG. 6, the second composition may be passed through a second microfluidic transfection device 114. The second microfluidic transfection device 114 may include an inlet configured to receive the second composition, a transfection component configured to transfect the second composition with the second exogenous molecule, thereby producing a second population of transfected cells, and an outlet configured to return the second population of transfected cells to the initial fluid medium 102. In an example, the second population of transfected cells may comprise the first sub-population of cells and the second sub-population of cells transfected with the same exogenous molecule (e.g., when the first exogenous molecule and the second exogenous molecule are the same exogenous molecule). In another example, the second population of transfected cells may comprise the first sub-population of cells transfected with the first exogenous molecule and the second sub-population of cells transfected with the second exogenous molecule (e.g., when the first exogenous molecule and the second exogenous molecule are different exogenous molecules). In some examples, the second population of transfected cells may include the first sub-population of cells transfected with the first exogenous molecule and the second exogenous molecule and the second sub-population of cells transfected with the second exogenous molecule.

In one aspect, the cells may have a successful transfection percentage of about 20% to about 25%, about 25% to about 30%, about 30% to about 35%, about 35% to about 40%, or more after being transfected using the second microfluidic transfection device 114. In an example, the cells may have a successful transfection percentage of about 20% to about 40% using the second microfluidic transfection device 114. In some examples, the second composition may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the second microfluidic transfection device 114. In an example, about 50% to about 90% of the cells may remain viable after being transfected using the second microfluidic transfection device 114.

The transfection component of the second microfluidic transfection device 114 may have various transfection parameters optimized for transfecting the second sub-population of cells as described herein. The transfection parameters may depend on the characteristics of the second sub-population of cells (e.g., size, stiffness, FACS characteristics, and/or other physical characteristics). In some examples, the second microfluidic transfection device 114 may be optimized specifically for the second sub-population of cells such that the first sub-population of cells is not transfected with the second exogenous molecule when passing through the second microfluidic transfection device. For example, the second exogenous molecule may be the same exogenous molecule as the first exogenous molecule and after the first population of transfected cells (e.g., first sub-population transfected with first exogenous molecule) and the second sub-population of cells are passed through the second microfluidic transfection device, the first sub-population of cells and the second sub-population of cells may both be transfected with only one exogenous molecule that is the same exogenous molecule.

As illustrated in FIG. 6, after the second sub-population of cells and/or the first population of transfected cells have been transfected by the second microfluidic transfection device 114, the second population of transfected cells (e.g., first population of transfected cells and the second sub-population transfected with the second exogenous molecule) are returned to the initial fluid medium 102 via the outlet in the second microfluidic transfection device 114.

The second population of transfected cells may be optionally held in the initial fluid medium 102 for a sufficient period of time (e.g., a holding period) to allow the second population of transfected cells to recover from the second transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period can allow the cells sufficient time and conditions to express one or more nucleotide sequences. In another example, the cells may be immediately injected with a third payload in the initial fluid medium 102 and have no holding period (e.g., no intervening culture step).

As illustrated in FIG. 6, the third sub-population of cells may be moved into the initial fluid medium (e.g., through the third flow path 606) and combined with the second population of transfected cells.

As illustrated in FIG. 6, the third payload reservoir 118 may be in fluid communication with the initial fluid medium 102 at a location after the second microfluidic transfection device 114. The third payload reservoir may provide a third exogenous molecule to the second population of transfected cells and the third sub-population of cells in the initial fluid medium 102. In an example, the third exogenous molecule, the second population of transfected cells, and the third sub-population of cells may be combined in the initial fluid medium to form a third composition. In one example, the third exogenous molecule may be a different exogenous molecule from the first exogenous molecule and/or second exogenous molecule. In another example, the first exogenous molecule, second exogenous molecule, and third exogenous molecule may be the same exogenous molecule. In a further example, the first exogenous molecule may be the same as the second exogenous molecule but different than the third exogenous molecule. In another example, the second exogenous molecule and the third exogenous molecule may be the same exogenous molecule, and the first exogenous molecule may be different from the second exogenous molecule and the third exogenous molecule.

The third composition may be passed through a third microfluidic transfection device (not shown) optimized for transfection of the third sub-population of cells, thereby producing a third composition of transfected cells comprising the first sub-population of cells transfected with the first exogenous molecule, the second sub-population of cells transfected with the second exogenous molecule, and the third sub-population of cells transfected with the third exogenous molecule. In some examples, the third microfluidic transfection device may be optimized specifically for the third sub-population of cells such that the second population of transfected cells (e.g., first sub-population with first exogenous molecule and second sub-population with second exogenous molecule) is not transfected with the third exogenous molecule when passing through the third microfluidic transfection device. For example, the third exogenous molecule may be the same exogenous molecule as the first and second exogenous molecules and after the second population of transfected cells (e.g., first sub-population transfected with first exogenous molecule and second sub-population with the second exogenous molecule) and the third sub-population of cells are passed through the third microfluidic transfection device, the first sub-population of cells, the second sub-population of cells, and the third sub-population of cells may all be transfected with only one exogenous molecule that is the same exogenous molecule.

The system may include a flow rate supply. In an example, the flow rate supply may be any device or instrument that provides a velocity to the cells in the system. In one example, the flow rate supply may be a pressure supply to apply a pressure of about 5 psi to about 100 psi to the system to move the cells between components. The pressure supply may comprise a regulator having one or multiple control systems. In an example, the regulator may provide pressure at locations where the cells need to move. The regulator may provide a pressure at a first location (e.g., at the cell sorter 402 before the first flow path 602) to move the first sub-population of cells through the first flow path 602, through the first exogenous molecule injection point, and through the first microfluidic transfection device 108 to a position connecting with the second flow path 604. The regulator may provide a pressure at a second location (e.g., at the cell sorter 402 before the second flow path 604) to move the second sub-population of cells through the second flow path 604, through the second exogenous molecule injection point, and then move both the first population of transfected cells and the second sub-population of cells through the second microfluidic transfection device 114. The regulator may provide a pressure at a third location (e.g., at the cell sorter 402 before the third flow path 606) to move the third population of cells through the third flow path 606, though the third exogenous molecule injection point, and then move both the second population of transfected cells and the third sub-population of cells through the third microfluidic transfection device.

In another aspect, the system may include multiple pressure supplies. Each pressure supply may have a regulator. In an example, a first pressure supply may have a regulator that may supply a pressure at a first location (e.g., at the cell sorter 402 before the first flow path 602) to move the first sub-population of cells through the first flow path 602, through the first exogenous molecule injection point, and through the first microfluidic transfection device 108 to a position connecting with the second flow path 604. A second pressure supply may have a regulator that may supply a pressure at a second location (e.g., at the cell sorter 402 before the second flow path 604) to move the second sub-population of cells through the second flow path 604, through the second exogenous molecule injection point, and then move both the first population of transfected cells and the second sub-population of cells through the second microfluidic transfection device 114. A third pressure supply may have a regulator that may supply a pressure at a third location (e.g., at the cell sorter 402 before the third flow path 606) to move the third population of cells through the third flow path 606, though the third exogenous molecule injection point, and then move both the second population of transfected cells and the third sub-population of cells through the third microfluidic transfection device. In some examples, one or the same pressure supply may be used to move the exogenous molecules from the first payload reservoir 106, the second payload reservoir 112, and the third payload reservoir 118 to the initial fluid medium 102.

The system may include N transfection devices and payload reservoirs configured to transfect N exogenous molecules into the cell or cells. The N holding chambers, transfection devices, and payload reservoirs may have the same structures, characteristics and properties as the first and second holding chambers, transfection devices, and payload reservoirs. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In an example, some or all of the transfection events may transfect the cells with the same exogenous molecule. In another example, the transfection events may transfect the cells with a different exogenous molecule.

The system 600 may be operable to conduct two or more transfection events sequentially. By transfecting the cells sequentially, groups of cells may be transfected quickly while retaining and/or improving on the successful transfection and viability rates of systems known in the art. The system 600 may be advantageous because it may be capable of successfully transfecting a heterogenous population of cells quickly without having to remove the cells from the system.

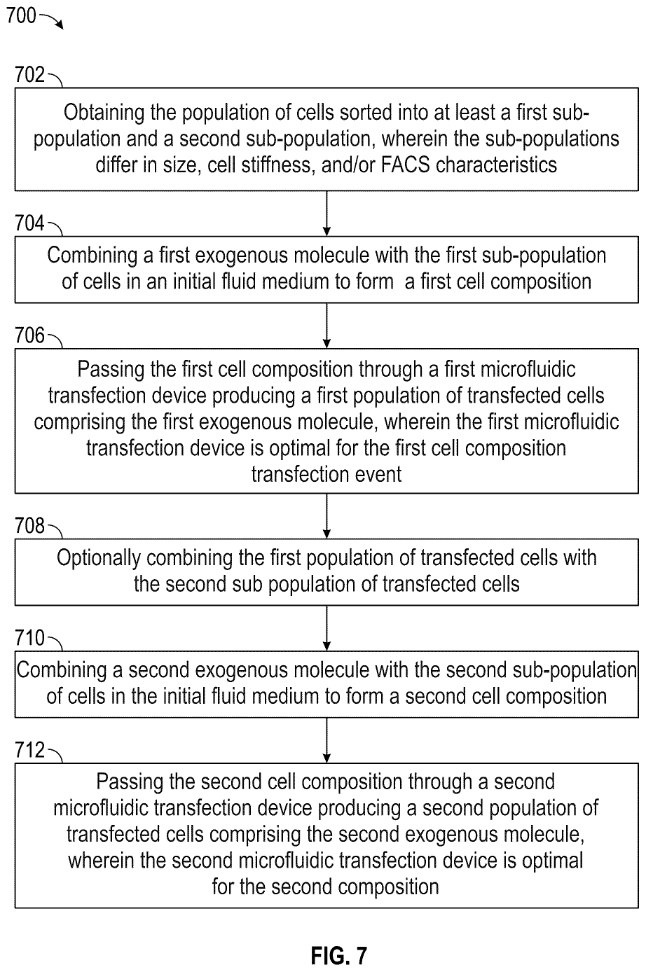

Further provided herein is a method for high throughput introduction of a plurality of exogenous molecules into a cell. The method 700, as illustrated in FIG. 7, may be configured to sort the cells into at least two groups (i.e., sub-populations). The method 700 may be operable to conduct two or more transfection events sequentially. By transfecting the cells sequentially, groups of cells may be transfected quickly while retaining and/or improving on the successful transfection and viability rates of methods known in the art. The method 700 may be advantageous because it may be capable of successfully transfecting a heterogenous population of cells quickly without having to remove the cells from a system. In some examples, the method 700 may be performed using the system 600.

As illustrated in FIG. 7, the method 700 may comprise one or more steps. At a first step 702, the method 700 may include obtaining the population of cells sorted into at least a first sub-population and a second sub-population, wherein the sub-populations differ in size, cell stiffness, FACS characteristics, and/or physical characteristics. In an example, the population of cells to be sorted may be a heterogenous population of cells. The FACS characteristics may be one or more of phenotype, receptor type, and/or frequency. In an example, the first sub-population of cells may have a cell diameter less than a diameter cut-off value. The second-sub population of cells may have a diameter greater than a diameter cut-off value.

In an aspect, the method 700 may include sorting the heterogenous population of cells into a first sub-population, a second sub-population, and a third sub-population. In an example, the heterogenous population of cells may be whole blood.

Sorting the cells may include sorting the cells into at least a first sub-population having an average cell diameter of about 10 μm to about 12 μm (neutrophils), a second sub-population having an average cell diameter of about 12 μm to about 15 μm (lymphocytes), and a third sub-population having an average cell diameter of about 15 μm to about 30 μm (monocytes).

As illustrated in FIG. 7, at a second step 704, the method 700 may include combining a first exogenous molecule with the first-sub population of cells in an initial fluid medium to form a first cell composition. The first exogenous molecule may be injected into the initial fluid medium along a first flow path by a first payload reservoir. The first sub-population of cells may be moved through the first flow path to combine with the first exogenous molecule using a flow rate supply (e.g., pressure supply).

As illustrated in FIG. 7, at a third step 706, the method 700 may include passing the first cell composition through a first microfluidic transfection device. Passing the first composition through the first microfluidic transfection device may produce a first population of transfected cells comprising the first exogenous molecule. The first microfluidic transfection device may be optimally tuned for the first composition transfection event as described herein.

The method 700 may include optionally holding the first population of transfected cells in the initial fluid medium for a holding period. In an example, the first population of transfected cells may be optionally held in the initial fluid medium for a sufficient period of time (e.g., a holding period) to allow the first population of transfected cells to recover from the first transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period can allow the cells sufficient time and conditions to express one or more nucleotide sequences. In another example, the first population of transfected cells may require no holding period before a second transfection event.

As illustrated in FIG. 7, at a fourth step 708, the method 700 may include optionally combining the first population of transfected cells with the second sub-population of cells. The second sub-population of cells may be moved from the cell sorter through the initial fluid medium along a second flow path to combine with the first population of transfected cells at a location in the initial fluid medium. The second sub-population of cells and the first population of transfected cells may be moved by supplying a flow rate using a flow rate supply (e.g., pressure supply).

As illustrated in FIG. 7, at a fifth step 710, the method 700 may include combining a second exogenous molecule with the second sub-population of cells, and optionally the first population of transfected cells, in the initial fluid medium to form a second cell composition. The second exogenous molecule may be injected into the initial fluid medium by a second payload reservoir. In some examples, the first exogenous molecule and the second exogenous molecule may be the same exogenous molecule. In another example, the first exogenous molecule and the second exogenous molecule may be different exogenous molecules.

As illustrated in FIG. 7, at a sixth step 712, the method 700 may include passing the second composition through a second microfluidic transfection device. Passing the second composition through the microfluidic transfection device may produce a second population of transfected cells comprising the second exogenous molecule. The second microfluidic transfection device may be optimally tuned for the second composition transfection event as described herein. In an example, the second microfluidic transfection device may have various transfection parameters for an optimal second transfection event. Optimizing the second microfluidic transfection device may be based on the type of cell and/or exogenous molecule in the second composition.

The transfection success rate and the viability of the cells after the first transfection (e.g., first sub-population and first exogenous molecule) and the second transfection (e.g., second sub-population and second exogenous molecule) may be the same as described herein.

The method 700 may include optionally holding the second population of transfected cells in the initial fluid medium for a holding period. In an example, the second population of transfected cells may be optionally held in the initial fluid medium for a sufficient period of time (e.g., a holding period) to allow the second population of transfected cells to recover from the second transfection event. In some examples, the holding period can allow the cells sufficient time and conditions to express one or more nucleotide sequences. In another example, the second population of transfected cells may require no holding period.

In one aspect, the steps of the method 700 may be repeated with a third sub-population of cells to produce a third population of transfected cells. In another example, the method 700 may include repeating the steps of the method N times to transfect the sub-populations of cells with N exogenous molecules. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events. The method 700, may allow for multiple sub-populations of cells to be transfected with exogenous molecules without removing the cells from the system.

Also presented herein is a method for high throughput introduction of a plurality of exogenous molecules into a heterogenous population of cells. The method may include passing the cell through N microfluidic processing cycles wherein N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Each cycle may include combining obtaining the population of cells sorted into at least a first sub-population and a second sub-population, combining a first exogenous molecule with the first sub-population of cells in an initial fluid medium to form a first cell composition, and passing the first cell composition through a microfluidic transfection device, thereby introducing the first exogenous molecule into the first sub-population of cells to form a first population of transfected cells comprising the first exogenous molecule. The cycle may further include combining a second exogenous molecule and optionally the first exogenous molecule with the second sub-population of cells and optionally the first population of transfected cells in the initial fluid medium to form a second cell composition, and passing the second cell composition through a microfluidic transfection device thereby introducing the second exogenous molecule and the optionally the first exogenous molecule into the second sub-population of cells and optionally the first population of transfected cells to form a second population of transfected cells comprising the second exogenous molecule and optionally the first exogenous molecule. The cycle may further include observing a holding period after each complete cycle or after each transfection event.

The transfection parameters of the transfection device may be tuned or optimized for each transfection event. In some examples, tuning the transfection device may include increasing or decreasing the gap size of the microfluidic transfection device based on the size, stiffness, FACS characteristics, or physical properties of the population of cells.

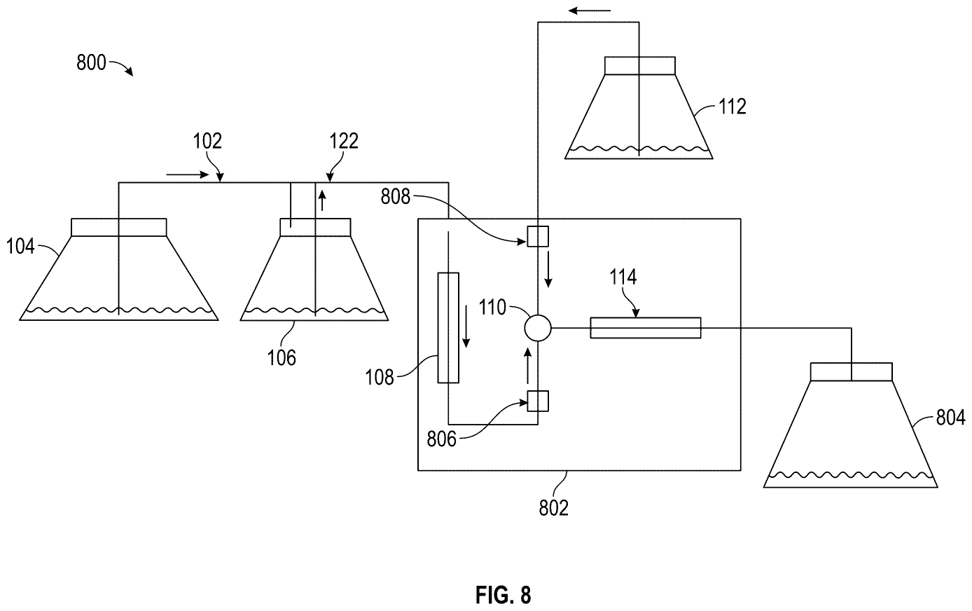

Further provided herein is a system for transfecting a plurality of cells with at least one exogenous molecule. The system 800 illustrated in FIG. 8 may be configured to sequentially transfect a plurality of cells. The sequential transfection can greatly reduce the time between successive transfection events compared to systems known in the art, while maintaining or improving the successful transfection rate and viability percentage of the cells.

As illustrated in FIG. 8, the system 800 may include a first reservoir 104, an initial fluid medium 102, a first payload reservoir 106, a microfluidic cassette 802, a second payload reservoir 112, and a collection reservoir 804. In some examples, the tubing 122 may contain the initial fluid medium 102. In other examples, the initial fluid medium 102 may be in the first reservoir 104 and transferred into the tubing 122 during operation. The first reservoir 104, first payload reservoir 106, microfluidic cassette 802, second payload reservoir 112, and collection reservoir 804 may be in fluid communication with the tubing 122. All the components of system 800 may be in fluid communication with the tubing 122.

As illustrated in FIG. 8, the first reservoir may be in fluid communication with the initial fluid medium 102. The first reservoir 104 may contain a plurality of cells. In an example, the plurality of cells may be homogenous. In another example, the plurality of cells may be heterogenous. The first reservoir 104 may be configured to provide the plurality of cells to the initial fluid medium 102. In an example, the plurality of cells may be moved from the first reservoir 104 to the initial fluid medium 102 by supplying a flow rate using a flow rate supply (e.g., pressure supply) to provide a cell velocity to the cells.

As illustrated in FIG. 8, the system 800 may include the first payload reservoir 106 in fluid communication with the initial fluid medium 102. The first payload reservoir 106 may be configured to inject a first exogenous molecule into the plurality of cells forming a first composition. The first payload reservoir 106 may be downstream from the first reservoir 104. The first exogenous molecule may be any of the exogenous molecules described herein.

As illustrated in FIG. 8, the initial fluid medium 102 may be in fluid communication with a microfluidic cassette 802. The microfluidic cassette 802 may include a first microfluidic transfection device 108, a second microfluidic transfection device 114, and a holding chamber 110. The microfluidic cassette 802 may include a first inlet in fluid communication with the initial fluid medium 102, a second inlet in fluid communication with a second payload reservoir 112, and an outlet in fluid communication with the collection reservoir 804.

The microfluidic cassette 802 may include two or more valves. In an example, a first valve 806 may be located between the first microfluidic transfection device 108 and the holding chamber 110. A second valve 808 may be located between a second payload reservoir 112 in fluid communication with the second inlet of the microfluidic cassette 802. The first valve 806 may have an open state and a closed state. The second valve 808 may have an open state and a closed state. In the open state, the first and second valves may allow cells or molecules to travel through the valves. In the closed state, the first and second valves may block cells or molecules from traveling through the valves.

The first microfluidic transfection device 108 may be in fluid communication with the first inlet of the microfluidic cassette 802 and in fluid communication with the holding chamber 110 of the microfluidic cassette 802. The first microfluidic transfection device 108 may be downstream of the first payload reservoir 106 and upstream of the holding chamber 110. The first microfluidic transfection device 108 may include an inlet configured to receive the first composition, a transfection component configured to transfect the plurality of cells with the first exogenous molecule, thereby producing a first population of transfected cells, and an outlet configured to return the first population of transfected cells to the holding chamber 110. In an example, the first population of transfected cells may comprise the first exogenous molecule.

The cell or cells may have a successful transfection percentage of about 20% to about 25%, about 25% to about 30%, about 30% to about 35%, about 35% to about 40%, or more after being transfected using the first microfluidic transfection device 108. In an example, the cell or cells may have a successful transfection percentage of about 20% to about 40% with the first exogenous molecule in the first microfluidic transfection device 108. In some examples, the first composition (e.g., cells and first exogenous molecule) may have a successful transfection percentage of about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the first microfluidic transfection device 108. In an example, about 50% to about 90% of the cells may remain viable after being transfected using the first microfluidic transfection device 108.

The transfection component of the first microfluidic transfection device 108 may have various transfection parameters optimized for transfecting the plurality of cells depending on the characteristics of the plurality of cells (e.g., size, stiffness, FACS characteristics, and/or physical characteristics) as described herein.

As illustrated in FIG. 8, the first valve 806 may be placed in an open state to move the first population of transfected cells to the holding chamber 110. The first population of transfected cells may be moved from the first microfluidic transfection device 108 to the holding chamber 110 by supplying a flow rate using the flow rate supply (e.g., pressure supply) to supply a cell velocity to the cells.

As illustrated in FIG. 8, the holding chamber 110 may be in fluid communication with the first microfluidic transfection device 108, the second microfluidic transfection device 114, and the second inlet of the microfluidic cassette 802. The holding chamber 110 may be downstream from the first microfluidic transfection device 108. The holding chamber 110 may be operable to receive the first population of transfected cells from the first microfluidic transfection device 108. In an example, the first population of transfected cells may be optionally held in the holding chamber 110 for a sufficient period of time (e.g., a holding period) to allow the first population of transfected cells to recover from the first transfection event. In some examples, the holding period may be about 0.1 seconds to about 60 minutes. In a further example, the holding period may be about 0.1 seconds to about 1 second, about 1 second to about 10 seconds, about 10 seconds to about 20 seconds, about 20 seconds to about 30 seconds, about 30 seconds to about 40 seconds, about 40 seconds to about 50 seconds, about 50 seconds to about 60 seconds, about 1 minute to about 5 minutes, about 5 minutes to about 10 minutes, about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, about 40 minutes to about 45 minutes, about 45 minutes to about 50 minutes, about 50 minutes to about 55 minutes, or about 55 minutes to about 60 minutes. In some examples, the holding period can allow the cells sufficient time and conditions to express one or more nucleotide sequences. In another example, the first population of transfected cells may be injected with a second exogenous molecule and passed through the second microfluidic transfection device 114 immediately (e.g., no intervening culture step).

As illustrated in FIG. 8, the second payload reservoir 112 may supply a second exogenous molecule to the holding chamber 110 through the second inlet of the microfluidic cassette 802 when the second valve 808 is in an open state by supplying a flow rate through a flow rate supply (e.g., pressure supply). The second exogenous molecule and the first population of transfected cells may form a second composition in the holding chamber 110.

In another aspect, the second payload reservoir 112 may also include a second population of cells to be moved to the holding chamber 110. The second payload reservoir 112 may inject the second population of cells and a second exogenous molecule into the holding chamber 110 when the second valve 808 is in an open state, forming a second composition with the first population of transfected cells. The second population of cells may have different characteristics from the plurality of cells transfected with the first exogenous molecule. The second population of cells may be of a different size, stiffness, adhesiveness, have different FACS characteristics, and/or have different physical properties. The first population of cells and second population of cells may comprise a heterogenous population of cells that are sorted (e.g., by a cell sorter) prior to the first plurality of cells being placed in the first reservoir 104. The first population and the second population can be sorted according to differences in average cell diameters, differences in cell stiffness, and/or differences in FACS characteristics.

As illustrated in FIG. 8, the second composition may move from the holding chamber 110 to the second microfluidic transfection device 114. The second microfluidic transfection device 114 may include an inlet configured to receive the second composition from the holding chamber 110, a transfection component configured to transfect the second composition with the second exogenous molecule, thereby producing a second population of transfected cells, and an outlet in fluid communication with the outlet of the microfluidic cassette 802 configured to move the second population of transfected cells to the collection reservoir 804.

The transfection component of the second microfluidic transfection device 114 may have various transfection parameters optimized for transfecting the cell or cells, depending on the characteristics of the cells in the second composition (e.g., cell size, stiffness, adhesiveness, FACS characteristics, and/or other physical properties) as described herein. In some examples, when a second population of cells is in the second composition, the second microfluidic transfection device 114 may be optimized for transfection of the second population of cells with the second exogenous molecule. In some examples, when the second composition only contains the first population of transfected cells and the second exogenous molecule, the second microfluidic transfection device may be optimized for the first population of transfected cells.

The second composition may have a successful transfection percentage of about 20% to about 25%, about 25% to about 30%, about 30% to about 35%, about 35% to about 40%, or more after being transfected using the second microfluidic transfection device 114. In an example, the cell or cells may have a successful transfection percentage of about 20% to about 40% with the second exogenous molecule in the second microfluidic transfection device 114. In some examples, the second composition (e.g., cells and second exogenous molecule) may have a successful transfection percentage of about 20% to about 25%, about 25% to about 30%, about 30% to about 35%, about 35% to about 40%, about 40% to about 45%, about 45% to about 50%, about 50% to about 55 %, about 55% to about 60%, about 60% to about 65%, about 65% to about 70%, about 70% to about 75%, about 75% to about 80%, or more. About 50% to about 55%, about 55% to about 60%, about 60% to about 65%, about 65% to about 70%, about 70% to about 75%, about 75% to about 80%, about 80% to about 85%, about 85% to about 90%, or more of the cells may remain viable after being transfected using the second microfluidic transfection device 114. In an example, about 50% to about 90% of the cells may remain viable after being transfected using the second microfluidic transfection device 114.

The system 800 may allow for high transfection success rates and high viability as compared to other systems known in the art due to the sequential nature of the system 800 (e.g., allowing a first transfection event and a second transfection event to occur with little time (e.g., less than 1 hour to about less than 24 hours) in between transfection events).

As illustrated in FIG. 8, the collection reservoir 804 may be operable to receive the second population of transfected cells from the microfluidic cassette 802. The collection reservoir 804 may be in fluid communication with the outlet of the microfluidic cassette 802. The collection reservoir 804 may be downstream of the microfluidic cassette.

The system may include a flow rate supply. In an example, the flow rate supply may be any device or instrument that provides a velocity to the cells in the system 800. In one example, the flow rate supply may be a pressure supply to apply a pressure of about 5 psi to about 100 psi, or any range therebetween, to the system 800 to move the cells between components.

The pressure supply may comprise a regulator having one or multiple control systems. In an example, the regulator may provide pressure at locations where the cells need to move. The regulator may supply pressure at a first location (e.g., the first reservoir 104) to move the cell or cells from the first reservoir 104 through the initial fluid medium 102, into the microfluidic cassette 802, through the first microfluidic transfection device 108, and into the holding chamber 110. The regulator may supply a pressure at a second location (e.g., the second payload reservoir 112) to move the second exogenous molecule from the second payload reservoir 112 through the second inlet of the microfluidic cassette 802 and into the holding chamber 110. The regulator may supply a pressure at a third location (e.g., the holding chamber 110) to move the second composition through the second microfluidic transfection device 114, through the outlet of the microfluidic cassette 802, and into the collection reservoir 804.

In another aspect, the system may include multiple pressure supplies. Each pressure supply may have a regulator. In an example, a first pressure supply may have a regulator that may supply a pressure at a first location (e.g., the first reservoir 104) to move the cell or cells from the first reservoir 104 through the initial fluid medium 102, into the microfluidic cassette 802, through the first microfluidic transfection device 108, and into the holding chamber 110. A second pressure supply may have a regulator that may supply a pressure at a second location (e.g., the second payload reservoir 112) to move the second exogenous molecule from the second payload reservoir 112 through the second inlet of the microfluidic cassette 802 and into the holding chamber 110. A third pressure supply may have a regulator that may supply a pressure at a third location (e.g., the holding chamber 110) to move the second composition through the second microfluidic transfection device 114, through the outlet of the microfluidic cassette 802, and into the collection reservoir 804.

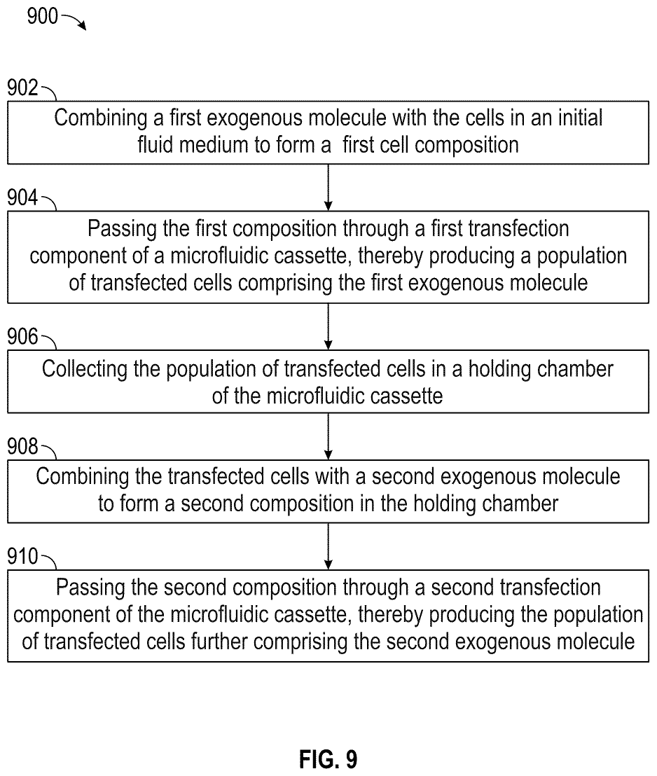

Also described herein is a method for high throughput introduction of a plurality of exogenous molecules to a plurality of cells. The method 900 illustrated in FIG. 9 may be configured to sequentially transfect a plurality of cells. The sequential transfection can greatly reduce the time between successive transfection events compared to methods known in the art, while maintaining or improving the successful transfection rate and viability percentage of the cells. The method 900 may be conducted using the system 800.

As illustrated in FIG. 9, the method 900 may begin at a first step 902. At the first step 902, the method 900 may include combining a first exogenous molecule with the plurality of cells in an initial fluid medium to form a first cell composition. The first exogenous molecule may be provided by a first payload reservoir. The first exogenous molecule may be any of the exogenous molecules described herein.

As illustrated in FIG. 9, at a second step 904, the method may include passing the first composition through a transfection component of a first microfluidic transfection device in a microfluidic cassette to produce a population of transfected cells comprising the first exogenous molecule. In an example, the transfection component of the first microfluidic transfection device may be tuned or optimized to transfect the plurality of cells with the first exogenous molecule. Tuning the transfection component may include increasing or decreasing the gap size or adjusting other transfection parameters. The first microfluidic transfection device may produce transfected cells having a transfection success rate and viability as described herein (e.g., the first composition may be transfected at a transfection success rate and viability as described herein).

As illustrated in FIG. 9, at a third step 906, the method 900 may include collecting the population of transfected cells in a holding chamber of the microfluidic cassette. The population of transfected cells may be held in the holding chamber for a sufficient period of time to allow recovery of the population of transfected cells.

As illustrated in FIG. 9, at a fourth step 908, the method 900 may include combining the transfected cells with a second exogenous molecule to form a second composition. The second exogenous molecule may be provided by a second payload reservoir in fluid communication with the holding chamber. In another example, the second payload reservoir may provide a second population of cells (e.g., a population of cells different from the transfected cells) and a second exogenous molecule to the holding chamber forming a second composition with the transfected cells. The second composition includes the second population of cells, the second exogenous molecule, and the transfected cells. The second exogenous molecule may be any exogenous molecule described herein.

As illustrated in FIG. 9, at a fifth step 910, the method 900 may include passing the second composition through a transfection component of a second microfluidic transfection device. Passing the second composition through the transfection component of the second microfluidic transfection device may produce a population of transfected cells comprising the second exogenous molecule. The transfection component of the second microfluidic transfection device may be tuned or optimized for transfection of the second composition with the second exogenous molecule and the first exogenous molecule. In an example, the tuning or optimizing the transfection component may include increasing or decreasing the gap size of the transfection component or adjusting other transfection parameters. The second microfluidic transfection device may produce transfected cells having a transfection success rate and viability as described herein (e.g., the second composition may be transfected at a transfection success rate and viability as described herein).

The population of transfected cells comprising the first and second exogenous molecules may be collected in a collection reservoir.

In another example, the method 900 may include repeating the steps of the method N times to transfect the sub-populations of cells with N exogenous molecules. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

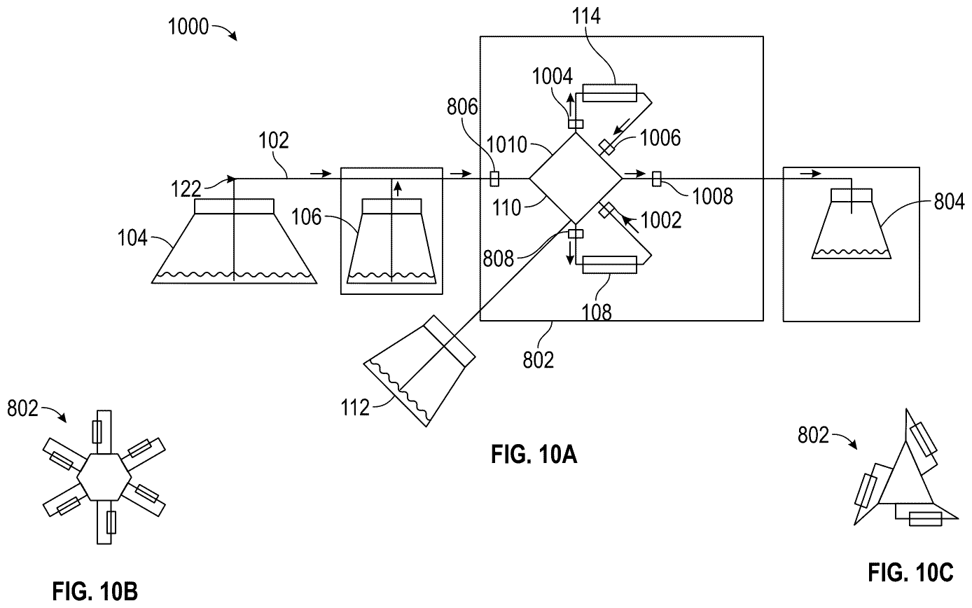

Further provided herein is a system for transfecting a plurality of cells with at least one exogenous molecule. The system 1000 illustrated in FIG. 10A may be configured to transfect a plurality of cells with at least one exogenous molecule. The system 1000 of FIG. 10A can be configured to sequentially transfect a plurality of cells with multiple exogenous molecules quickly, while maintaining the same or better rates of successful transfection and viability compared to other systems known in the art. The system of FIG. 10A is configured to produce multiple transfection events within a short amount of time (e.g., sequential transfection events may occur within the same hour).

The system 800 of FIG. 8 may be alternatively configured as illustrated in FIG. 10A. The system 1000 may include the first reservoir 104, the first payload reservoir 106, the holding chamber 110, the microfluidic cassette 802 comprising the first microfluidic transfection device 108, the second microfluidic transfection device 114, the first valve 806, the second valve 808, and the collection reservoir 804. In some examples, the tubing 122 may contain the initial fluid medium 102. In other examples, the initial fluid medium 102 may be in the first reservoir 104 and transferred into the tubing 122 during operation. The first reservoir 104, first payload reservoir 106, microfluidic cassette 802, second payload reservoir 112, holding chamber 110, first valve 806, second valve 808, and collection reservoir 804 may be in fluid communication with the tubing 122. All the components of system 1000 may be in fluid communication with the tubing 122.

As illustrated in FIGS. 10B and 10C, the microfluidic cassette 802 may include more than two microfluidic transfection devices. The microfluidic cassette may include three, four, five, six, seven, eight, nine, or ten microfluidic transfection devices. Each microfluidic transfection device may be tuned or optimized for different cell types or exogenous molecule types as discussed above.

As illustrated in FIG. 10A, the microfluidic transfection device may have a plurality of valves. A first valve 806 may be located on the initial fluid medium 102 and in fluid communication with the first reservoir 104, the first inlet of the microfluidic cassette 802, and the holding chamber 110. The first valve 806 may have an open state and a closed state. A second valve 808 may be located between the holding chamber 110 and the first microfluidic transfection device 108. The second valve 808 may have an open state and a closed state. A third valve 1002 may be located between the first microfluidic transfection device 108 and the holding chamber 110. A fourth valve 1004 may be located between the holding chamber 110 and the second microfluidic transfection device 114. A fifth valve 1006 may be located between the second microfluidic transfection device 114 and the holding chamber 110. A sixth valve 1008 may be located on a microfluidic cassette outlet path between the holding chamber 110 and the microfluidic cassette outlet. The first, second, third, fourth, fifth, and sixth valves may be control valves having an open state and a closed state. One or more of the valves may be opened to direct the cells to a desired location. In an example, only one valve is in an open state at a time. In another example, two valves may be in an open state at one time. The system 1000 may have more than six valves when the system 1000 has more than two microfluidic transfection devices.

The first valve 806 may be in an open state to allow the cells to enter the holding chamber 110 from the first inlet of the microfluidic cassette 802. Once the cells have entered the holding chamber 110 the first valve 806 may be placed in a closed state. In an example, the second valve 808 may be placed in an open state. A flow rate (e.g., via a flow rate supply such as a pressure supply 1010) may be supplied to the holding chamber 110 to move the cells to the inlet of the first microfluidic transfection device. When the second valve 808 is in an open state the cells may move through the first microfluidic transfection device 108 to produce a first population of transfected cells. The second valve 808 may be placed in a closed state after the cells have traveled through the first microfluidic transfection device 108. The third valve 1002 may then be placed in an open state to allow the first population of transfected cells to move from the first microfluidic transfection device 108 through the first transfection device outlet path back to the holding chamber 110. The third valve 1002 may then be placed in a closed state.

In one aspect, the system may not include the first payload reservoir 106. The cells may flow directly into the holding chamber 110 from the first reservoir 104 through the first valve 806 in an open state. The cells may be combined with a first exogenous molecule in the holding chamber 110 to form a first composition. The first exogenous molecule may be provided by the second payload reservoir 112. The first exogenous molecule may be any of the exogenous molecules described herein.

Once the first population of transfected cells have returned to the holding chamber 110, the second payload reservoir 112 may supply the first population of transfected cells with a second exogenous molecule forming a second composition. The second exogenous molecule may be any of the exogenous molecules described herein.

A flow rate (e.g., via a flow rate supply such as a pressure supply 1010) may be supplied to the holding chamber 110 to move the second composition to the inlet of the second microfluidic transfection device 114. When the fourth valve 1004 is in an open state the cells may move through the second microfluidic transfection device 114 to produce a second population of transfected cells. The fourth valve 1004 may be placed in a closed state after the second composition has traveled through the second microfluidic transfection device 114. The fifth valve 1006 may then be placed in an open state to allow the second population of transfected cells to move from the second microfluidic transfection device 114 back to the holding chamber 110. The fifth valve 1006 may then be placed in a closed state.

In one aspect, to remove the cells from the microfluidic cassette 802, a flow rate may be applied to the cells in the holding chamber and the sixth valve 1008 may be placed in an open state. The cells may then flow through the sixth valve 1008 and out of the microfluidic cassette 802 into the collection reservoir 804. In another aspect, the cells may be removed through the microfluidic cassette by supplying a flow rate to the holding chamber, placing the first valve 806 in an open state, and allowing the cells to flow back into the first reservoir 104.

As illustrated in FIG. 10A, after the cells are transfected with an exogenous molecule in one of the microfluidic transfection devices (e.g., first microfluidic transfection device 108 and second microfluidic transfection device 114) they may return to the holding chamber 110. The cells may be held in the holding chamber 110 for a holding period as described above. In another example, the cells may be immediately injected with another exogenous molecule and then passed through another transfection device (e.g., no intervening culture step). The payload (e.g., exogenous molecule) may be replaced between each transfection event in the second payload reservoir 112. This process may be repeated N times with N microfluidic transfection devices and N exogenous molecules until the cells have been transfected a desired number of times. In some examples, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In an example, the exogenous molecules may be the same throughout some or all of the transfection events to produce a higher transfection percentage. In another example, the exogenous molecules may be different for each transfection event.

The system 1000 may exhibit the same high transfection success rates and high cell viabilities as described herein for all of the transfection events.

A single flow rate supply (e.g., pressure supply 1010) may be provide a flow rate (e.g., via a pressure) to the holding chamber 110 to move the cells through the first microfluidic transfection device 108 and the second microfluidic transfection device 114. The valves may be operable to direct the cells to the first microfluidic transfection device 108 or second microfluidic transfection device 114. The flow rate supply may be any of the flow rate supplies described herein.

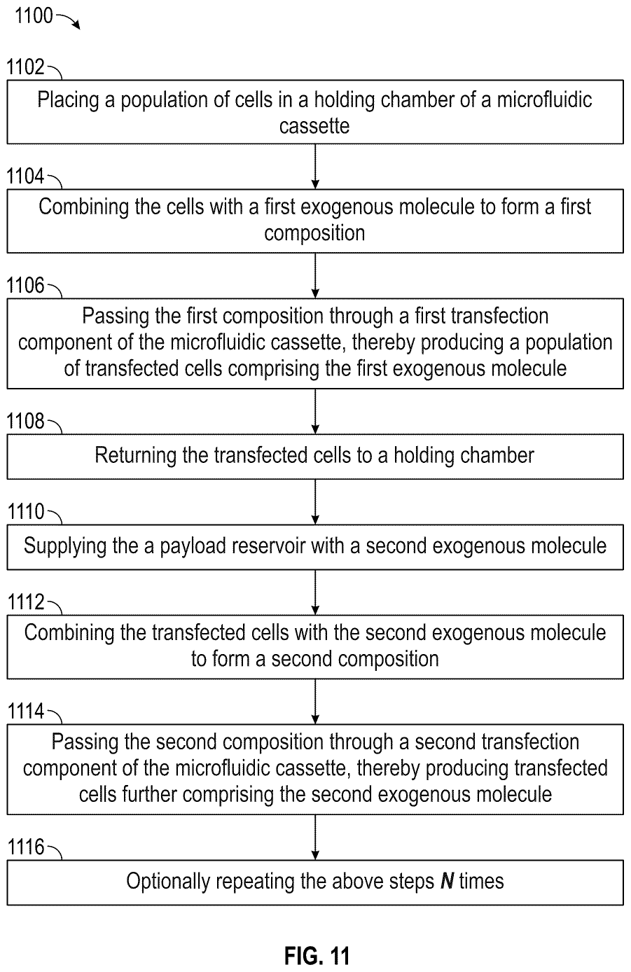

Also provided herein is a method for transfecting a plurality of cells with a plurality of exogenous molecules. The method 1100, as illustrated in FIG. 11, may provide a sequential transfection of a plurality of cells with a plurality of exogenous molecules rapidly. The method may provide transfection of a plurality of cells with a plurality of exogenous molecules much quicker than methods known in the art, while maintaining the same or better successful transfection rates and viability percentages. The method 1100 may be performed using the system 1000.

As illustrated in FIG. 11, at a first step 1102, the method 1100 may begin by placing the cells in a holding chamber of a microfluidic cassette. A first valve of the microfluidic cassette may be placed in an open state to allow the cells to flow into a first inlet of the holding chamber. The cells may be provided with a flow rate via a flow rate supply (e.g., a pressure supplied by a pressure supply). After the cells have all moved into the holding chamber, the first valve may be placed in a closed state.

As illustrated in FIG. 11, at a second step 1104, the method 1100 may include combining the cells with a first exogenous molecule to form a first composition. The first exogenous molecule may be independently selected from the exogenous molecules described herein. The first exogenous molecule may be combined with the cells in the holding chamber by providing a flow rate (e.g., via a pressure from a pressure supply) to a payload reservoir to move the first exogenous molecule to the holding chamber. The first exogenous molecule can flow along a flow path to a second inlet of the holding chamber.

As illustrated in FIG. 11, at a third step 1106, the method 1100 may include passing the first composition through a first microfluidic transfection device of the microfluidic cassette. By passing the first composition through the first microfluidic transfection device, a first population of transfected cells comprising the first exogenous molecule may be produced. To pass the cells through the first microfluidic transfection device, a flow rate may be supplied to the holding chamber (e.g., a pressure via a pressure supply). A second valve may be placed in an open state on a flow path from the holding chamber to the first microfluidic transfection device to allow the first composition to enter the inlet of the first microfluidic transfection device. The flow rate may push the first composition through the microfluidic component of the first microfluidic transfection device and out the outlet of the first microfluidic transfection device. After the first composition has passed through the inlet of the first microfluidic transfection device, the second valve may be placed in a closed state. A third valve on a first transfection device outlet path may then be placed in an open state to allow the first population of transfected cells to move to the holding chamber through a third inlet of the holding chamber. In an example, the microfluidic component of the first microfluidic transfection device may be tuned or optimized for the transfection of the first composition. In some examples, tuning or optimizing the microfluidic transfection component may include increasing or decreasing the gap size.

As illustrated in FIG. 11, at a fourth step 1108, the method 1100 may include returning the first population of transfected cells to the holding chamber. In an example, the first population of transfected cells may be optionally held in the holding chamber for a sufficient period of time (e.g., a holding period) to allow the first population of transfected cells to recover from the first transfection event. In another example, the cells may be immediately injected with a second payload when they reach the holding chamber and have no holding period (e.g., no intervening culture step).

As illustrated in FIG. 11, at a fifth step 1110, the method 1100 may include suppling the payload reservoir with a second exogenous molecule. The payload reservoir may be easily reloaded with the second exogenous molecule. In another example, the payload reservoir may be removed and replaced with a new payload reservoir containing the second exogenous molecule. In some examples, the payload reservoir may contain a new (e.g., different population) of cells and the second exogenous molecule.

As illustrated in FIG. 11, at a sixth step 1112, the method 1100 may include combining the population of transfected cells with the second exogenous molecule to form a second composition. A flow rate (e.g., via a pressure from a pressure supply) can be supplied to the payload reservoir to move the second exogenous molecule from the payload reservoir to the holding chamber. The second exogenous molecule may be independently selected from the exogenous molecules described herein. The second exogenous molecule may be combined with the cells in the holding chamber by providing a flow rate (e.g., via a pressure from a pressure supply) to a payload reservoir to move the second exogenous molecule to the holding chamber.

As illustrated in FIG. 11, at a seventh step 1114, the method 1100 may include passing the second composition through a second microfluidic transfection device of the microfluidic cassette. By passing the second composition through the second microfluidic transfection device, a second population of transfected cells is produced comprising the second exogenous molecule and the first exogenous molecule. A flow rate may be provided to the holding chamber to move the second composition through an inlet flow path of the second microfluidic transfection device. A fourth valve located on the inlet flow path of the second microfluidic device may be placed in an open state to allow the second composition to flow into the inlet of the second microfluidic transfection device. Once the second composition is fully into the microfluidic component of the second microfluidic transfection device, the fourth valve may be placed in a closed position. The second composition may then be passed through the microfluidic transfection component of the second microfluidic transfection device. In some examples, the microfluidic transfection component may be tuned or optimized for transfection of the second composition. In some examples, tuning or optimizing the transfection component may comprise increasing or decreasing the gap size or adjusting other transfection parameters. The second population of transfected cells may then be returned to the holding chamber. A fifth valve may be placed in an open state to allow the second population of transfected cells to flow to from the outlet of the second microfluidic transfection device along a second microfluidic transfection device outlet path and into the holding chamber.

The second population of transfected cells may be optionally held in the holding chamber for a sufficient period of time (e.g., a holding period) to allow the second population of transfected cells to recover from the second transfection event. In some examples, the holding period can allow the cells sufficient time and conditions to express one or more nucleotide sequences.

The transfection devices described with respect to method 1100 may be optimized in the same manner as described herein. The transfection events produced by the method 1100 may have the same transfection success rates and cell viabilities as described herein.

At an eighth step 1116, the method 1100 may include repeating the steps N times. N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. The microfluidic cassette may have N microfluidic transfection devices and two times N valves. The method may be repeated to produce a desired number of transfection events. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

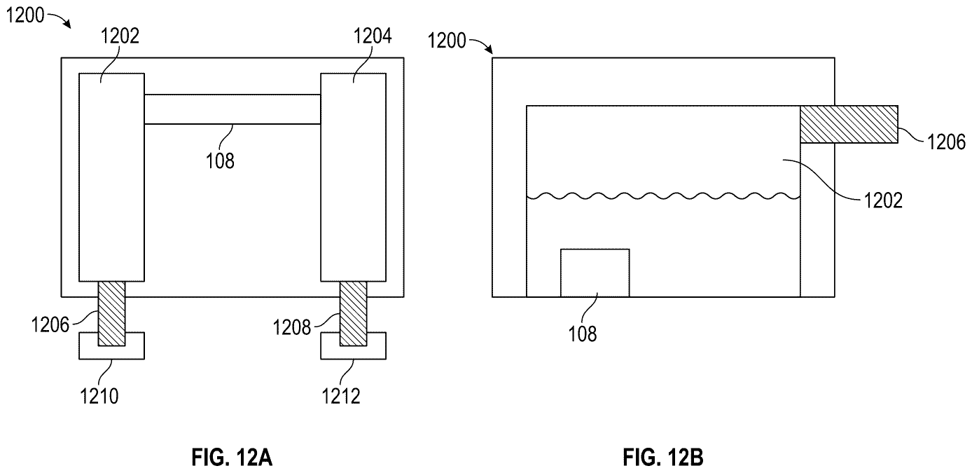

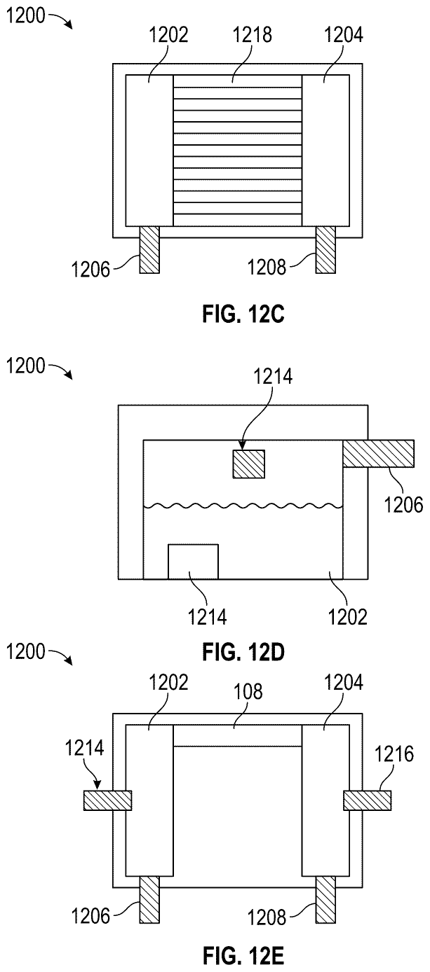

Further provided herein is a system for transfecting a plurality of cells with one or more exogenous molecules. The system 1200 as illustrated in FIG. 12A-E may be designed to transfect a plurality of cells with one or more exogenous molecules. The system 1200 may be operable to rapidly transfect the cells with the exogenous molecules much quicker than systems known in the art. Further, the system 1200 may be designed to transfect the cells with only one exogenous molecule multiple times, greatly increasing the successful transfection rate.

As illustrated in FIG. 12A, the system 1200 may be a microfluidic consumable which may include a first reservoir 1202, a second reservoir 1204, a first flow rate supply 1206, a second flow rate supply 1208, and a microfluidic transfection device 108.

As illustrated in FIG. 12A-B, the microfluidic consumable may contain a first reservoir 1202. The first reservoir 1202 may be in fluid communication with a first flow rate supply 1206, a first valve 1210, and a microfluidic transfection device 108. The first reservoir 1202 may be operable to receive a first composition comprising a plurality of cells and a first exogenous molecule. The first exogenous molecule may be any of the exogenous molecules described herein.

As illustrated in FIG. 12A-B, first reservoir 1202 may include a first flow rate supply 1206. In an example, the first flow rate supply 1206 may be any device or instrument that provides a velocity to the cells in the system 1200. In one example, the first flow rate supply may be a first pressure supply to apply a pressure of about 5 psi to about 100 psi to the system to move the cells from the first reservoir through the microfluidic transfection device to the second reservoir. The first flow rate supply 1206 may be any of the flow rate supplies described herein.

As illustrated in FIG. 12A-B, the system 1200 may include a first valve 1210. The first valve 1210 may have an open and a closed state. The first valve 1210 may be placed in an open state to allow the first flow rate supply 1206 to provide a flow rate to the first composition to move the first composition from the first reservoir through the microfluidic transfection device 108 to the second reservoir 1204. The first valve 1210 may be placed in a closed state when the first composition has passed through the microfluidic transfection device 108 to the second reservoir 1204.

As illustrated in FIG. 12A-B, the system may include a microfluidic transfection device 108. The microfluidic transfection device 108 may comprise a first inlet, a transfection component, and a second inlet. The first inlet may be operable to receive the composition from the first reservoir 1202 in one direction and to receive transfected cells from the transfection component in the opposite direction. The second inlet may be operable to receive transfected cells from the transfection component in one direction and operable to receive a composition from the second reservoir 1204 in an opposite direction.

The transfection component may be operable to transfect the cells of the first composition with the first exogenous molecule to produce a first population of transfected cells.

The transfection component may be tuned or optimized for the transfection of cells based on the type of cells (e.g., size, stiffness, FACS characteristics, and/or physical characteristics) to be transfected as described herein.

As illustrated in FIG. 12A-B, the system 1200 may include a second reservoir 1204. The second reservoir 1204 may be in fluid communication with a second flow rate supply 1208, a second valve 1212, and a microfluidic transfection device 108. The second reservoir 1204 may be operable to receive the first population of transfected cells.

As illustrated in FIG. 12A-B, second reservoir 1204 may include a second flow rate supply 1208. In an example, the second flow rate supply 1208 may be any device or instrument that provides a velocity to the cells in the system. In one example, the second flow rate supply 1208 may be a second pressure supply to apply a pressure of about 5 psi to about 100 psi to the system 1200 to move the cells from the second reservoir 1204 through the microfluidic transfection device 108 to the first reservoir 1202. The second flow rate supply 1208 may be any of the flow rate supplies described herein.

As illustrated in FIG. 12A-B, the system 1200 may include a second valve 1212. The second valve 1212 may have an open and a closed state. The second valve 1212 may be placed in an open state to allow the second flow rate supply 1208 to provide a flow rate to the first population of transfected cells to move the first population of transfected cells from the second reservoir 1204 through the microfluidic transfection device 108 to the first reservoir 1202. The second valve 1212 may be placed in a closed state when the first population of transfected cells has passed through the microfluidic transfection device 108 to the first reservoir 1202.

Passing the first population of transfected cells through the transfection component a second time may increase the successful transfection percentage (e.g., by transfecting the cells with the same exogenous molecule twice). In other examples, the first reservoir 1202 may have a first injector inlet in fluid communication with a first payload reservoir 1214, as illustrated, for example, in FIGS. 12D and 12E. The second reservoir 1204 may have a second injector inlet in fluid communication with a second payload reservoir 1216. The first payload reservoir 1214 may be operable to inject an exogenous molecule into the cells in the first reservoir 1202 and the cells may be transfected with the exogenous molecule by being passed through the microfluidic transfection device 108. The second payload reservoir 1216 may be operable to inject an exogenous molecule into the cells in the second reservoir 1204 and the cells may be transfected with the exogenous molecule by being passed through the microfluidic transfection device 108.

As illustrated in FIG. 12C, the system 1200 may be a high throughput microfluidic consumable. The high throughput microfluidic consumable may have multiple microfluidic transfection components 1218.

The system 1200 may have the same high transfection success rates and high cell viabilities as described herein for all transfection events.

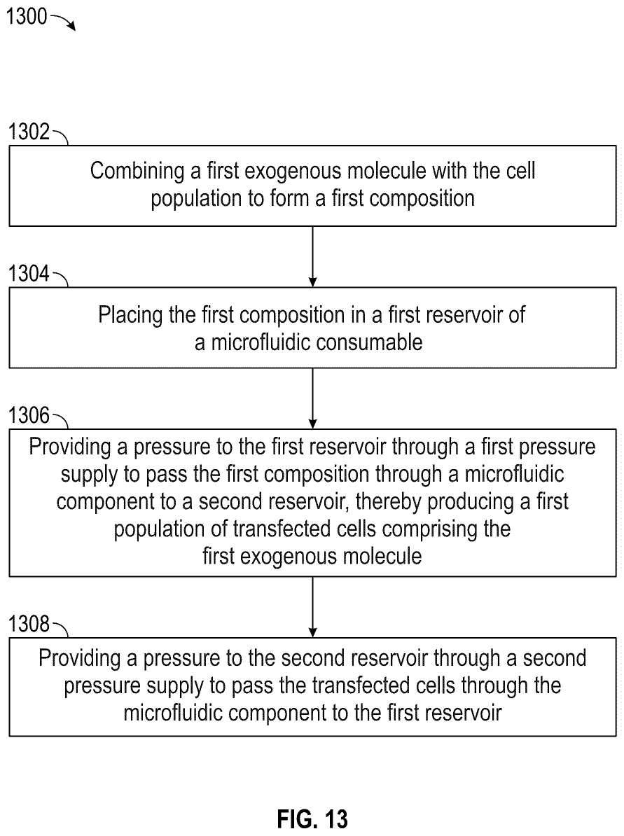

Also described herein is a method for transfecting a plurality of cells with at least one exogenous molecule. The method 1300 as illustrated in FIG. 13 may be designed to transfect a plurality of cells with one or more exogenous molecules. The method 1300 may rapidly transfect the cells with the exogenous molecules much quicker than systems known in the art. Further, the method 1300 may be designed to transfect the cells with only one exogenous molecule multiple times, greatly increasing the successful transfection rate. The method 1300 may be conducted using the system 1200.

As illustrated in FIG. 13, the method 1300 may begin at a first step 1302. At the first step 1302, the method 1300 may include combining a first exogenous molecule with a cell population to form a first composition. The first exogenous molecule may be any of the exogenous molecules described herein.

As illustrated in FIG. 13, at a second step 1304, the method 1300 may include placing the first composition in the first reservoir of a microfluidic consumable.

A microfluidic transfection component in fluid communication with the first reservoir and a second reservoir may be tuned based on the size, stiffness, FACS characteristics, or other physical properties of the cells to optimize the transfection of the cells with the first exogenous molecule as described herein. The transfection component may be tuned by increasing or decreasing the gap size, or as otherwise discussed above.

As illustrated in FIG. 13, at a third step 1306, the method 1300 may include providing a pressure to the first reservoir through a first pressure supply to pass the first composition through a microfluidic component to a second reservoir producing a first population of transfected cells comprising the first exogenous molecule.

As illustrated in FIG. 13, at a fourth step 1308, the method 1300 may include providing a pressure to the second reservoir through a second pressure supply to pass the transfected cells through the microfluidic component to the first reservoir. In some examples, before the transfected cells are passed through the microfluidic component to the first reservoir, a second exogenous molecule can be added to the transfected cells.

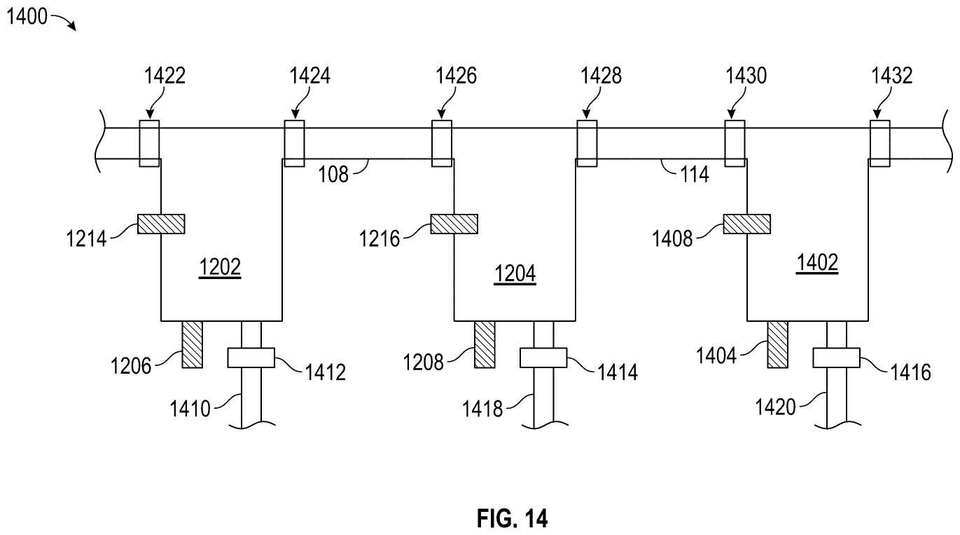

Also presented herein is a system for transfecting a plurality of cells with at least one exogenous molecule. The system 1400 of FIG. 14 may be configured to transfect a plurality of cells with a plurality of exogenous molecules sequentially. The system 1400 may be self-contained and capable of providing a sample after each transfection event. Further, the system 1400 may be operable to produce multiple transfection events much quicker than systems known in the art, while maintaining the same or better successful transfection rates and viability percentages. It will be appreciated that the sample collection mechanism of system 1400 can be incorporated in any of the systems and methods described herein.

As illustrated in FIG. 14, the system 1400 may have many of the components of the system of FIGS. 12A-E. As illustrated in FIG. 14, the system 1400 may include a first reservoir 1202, a second reservoir 1204, a third reservoir 1402, a first microfluidic transfection device 108, and a second microfluidic transfection device 114.

As illustrated in FIG. 14, the first reservoir may include a first cell inlet, a first cell inlet valve 1422, a first flow rate supply 1206, a first sample port 1410, a first payload reservoir 1214, a first sample port valve 1412, and an outlet (e.g., in fluid communication with the inlet valve 1424 of the first microfluidic transfection device 108). The first cell inlet may provide a plurality of cells to the first reservoir 1202. The first payload reservoir 1214 may provide a first exogenous molecule to the plurality of cells in the first reservoir 1202, thereby forming a first composition in the first reservoir 1202.

The first flow rate supply 1206 may be any device or instrument that provides a velocity to the cells in the system. In one example, the first flow rate supply 1206 may be a first pressure supply to apply a pressure of about 5 psi to about 100 psi to the system to move the cells from the first reservoir 1202 through the first microfluidic transfection device 108 to the second reservoir 1204. The first flow rate supply 1206 may be any of the flow rate supplies described herein.

As illustrated in FIG. 14, the first reservoir 1202 may have a first sample port 1410. In an example, the first sample port 1410 may be operable to remove a sample of the plurality of cells to test the cells. The first sample port 1410 may be connected to a first cell testing device. In an example, the first cell testing device (e.g., analytical instrument) may be a flow cytometer, a cell counter, a next generation sequencing instrument, or other analytical instrument capable of analyzing the transfected cells configured to conduct an analysis of the sample. The first sample port 1410 may have a first sample port valve 1412 having an open state and a closed state. When a sample is to be removed the first sample port valve 1412 may be in an open state. A flow rate may be supplied by the first flow rate supply 1206 to the first reservoir 1202 to move a sample of the plurality of cells through the first sample port valve 1412 and into the first cell testing device. Once the sample has been removed, the first sample port valve 1412 may be placed in the closed state.

As illustrated in FIG. 14, the system 1400 may have a first microfluidic transfection device 108. The first microfluidic transfection device 108 may comprise an inlet, an inlet valve 1424, a transfection component, an outlet, and an outlet valve 1426. The inlet valve may be configured to have an open state and a closed state. The inlet valve may be placed in an open state and a flow rate may be supplied to the first reservoir to move the first composition from the first reservoir to the first transfection component. Once the first composition has passed into the transfection component, the first inlet valve may be placed in a closed state.

The transfection component of the first microfluidic transfection device 108 may be operable to transfect the cells of the first composition with the first exogenous molecule to produce a first population of transfected cells. The transfection component of the first microfluidic transfection device 108 may be tuned or optimized for the transfection of cells based on the type of cells (e.g., size, stiffness, FACS characteristics, or physical characteristics) to be transfected as described herein.

As illustrated in FIG. 14, the outlet valve of the first microfluidic transfection device 108 may be placed in an open state to allow the first population of transfected cells to move through the first microfluidic transfection device 108 into the second reservoir 1204.

As illustrated in FIG. 14, the second reservoir 1204 may include a second cell inlet, a second flow rate supply 1208, a second sample port 1418, a second payload reservoir 1216, a second sample port valve 1414, and an outlet. The second cell inlet may allow the first population of transfected cells to enter into the second reservoir 1204. The second payload reservoir 1216 may provide a second exogenous molecule to the first population of transfected cells in the second reservoir 1204, thereby forming a second composition in the second reservoir 1204. The second exogenous molecule may be any of the exogenous molecules described herein.

The second flow rate supply 1208 may be any device or instrument that provides a velocity to the cells in the system. In one example, the second flow rate supply 1208 may be a second pressure supply to apply a pressure of about 5 psi to about 100 psi to the system 1400 to move the cells from the second reservoir 1204 through the second microfluidic transfection device 114 to the third reservoir 1402. The second flow rate supply 1208 may be any of the flow rate supplies described herein.

The first population of transfected cells may be optionally held in the second reservoir 1204 for a sufficient period of time (e.g., a holding period) to allow the first population of transfected cells to recover from the first transfection event. In some examples, the holding period can allow the cells sufficient time and conditions to express one or more nucleotide sequences. In another example, the cells may be immediately injected with a second payload when they reach the second reservoir and have no holding period (e.g., no intervening culture step).

As illustrated in FIG. 14, the second reservoir 1204 may have a second sample port 1418. In an example, the second sample port 1418 may be operable to remove a sample of the first population of transfected cells to test the cells during the holding period. The second sample port 1418 may be connected to a second cell testing device. In an example, the second cell testing device (e.g., analytical instrument) may be a flow cytometer, a cell counter, a next generation sequencing instrument, or other analytical instrument capable of analyzing the transfected cells. The cell testing device may be operable to analyze whether the cells were successfully transfected and the viability of the cells that were successfully transfected. The second sample port 1418 may have a second sample port valve 1414 having an open state and a closed state. When a sample is to be removed the second sample port valve 1414 may be in an open state. A flow rate may be supplied by the second flow rate supply 1208 to the second reservoir 1204 to move a sample of the first population of transfected cells through the second sample port valve 1414 and into the second cell testing device. Once the sample is removed, the second sample port valve 1414 may be placed in a closed state.

As illustrated in FIG. 14, the system 1400 may have a second microfluidic transfection device 114. The second microfluidic transfection device 114 may comprise a second inlet, a second inlet valve 1428, a transfection component, a second outlet, and a second outlet valve 1430. The second inlet valve 1428 may be configured to have an open state and a closed state. The second inlet valve 1428 may be placed in an open state and a flow rate may be supplied to the second reservoir 1204 to move the second composition from the second reservoir 1204 to the second microfluidic transfection device 114. Once the second composition has passed into the second microfluidic transfection component the second inlet valve 1428 may be placed in a closed state.

The transfection component of the second microfluidic transfection device 114 may be operable to transfect the cells of the second composition with the second exogenous molecule to produce a second population of transfected cells. The transfection component of the second microfluidic transfection device 114 may be tuned or optimized for the transfection of cells based on the type of cells (e.g., size, stiffness, FACS characteristics, or other physical characteristics) to be transfected as described herein.

As illustrated in FIG. 14, the second outlet valve 1430 of the second microfluidic transfection device 114 may be placed in an open state to allow the second population of transfected cells to move through the second microfluidic transfection device 114 into the third reservoir 1402.

As illustrated in FIG. 14, the third reservoir 1402 may include a third cell inlet, a third flow rate supply 1404, a third sample port 1420, a third payload reservoir 1408, a third sample port valve 1416, and an outlet. The third cell inlet may allow the second population of transfected cells to enter into the third reservoir 1402. The third payload reservoir 1408 may provide a third exogenous molecule to the second population of transfected cells in the third reservoir 1402, thereby forming a third composition in the third reservoir 1402.

The third flow rate supply 1404 may be any device or instrument that provides a velocity to the cells in the system. In one example, the third flow rate supply 1404 may be a third pressure supply to apply a pressure of about 5 psi to about 100 psi to the system 1400 to move the third composition from the third reservoir 1402 through the microfluidic transfection device to a fourth reservoir. The third flow rate supply 1404 may be any of the flow rate supplies described herein.

The second population of transfected cells may be optionally held in the third reservoir 1402 for a sufficient period of time (e.g., a holding period) to allow the second population of transfected cells to recover from the second transfection event. In another example, the cells may be immediately injected with a third payload when they reach the third reservoir and have no holding period (e.g., no intervening culture step). In some examples, the holding period can allow the cells sufficient time and conditions to express one or more nucleotide sequences.

As illustrated in FIG. 14, the third reservoir 1402 may have a third sample port 1420. In an example, the third sample port 1420 may be operable to remove a sample of the second population of transfected cells to test the cells during the holding period. The third sample port 1420 may be connected to a third cell testing device. In an example, the third cell testing device (e.g., analytical instrument) may be a flow cytometer, a cell counter, a next generation sequencing instrument, or other analytical instrument capable of analyzing the transfected cells. The cell testing device may be operable to analyze whether the cells were successfully transfected and the viability of the cells that were successfully transfected. The third sample port 1420 may have a third sample port valve 1416 having an open state and a closed state. When a sample is to be removed the third sample port valve 1416 may be in an open state. A flow rate may be supplied by the third flow rate supply 1404 to the third reservoir 1402 to move a sample of the second population of transfected cells through the third sample port valve 1416 and into the third cell testing device. Once the sample is removed, the third sample port valve 1416 may be placed in a closed state.

The system may include N reservoirs and N microfluidic transfection devices to transfect the plurality of cells with N exogenous molecules in N transfection events. In an example, N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be used in all or some of the N transfection events.

The system 1400 may be operable to produce transfection events having the same transfection success rates and cell viabilities as described herein.

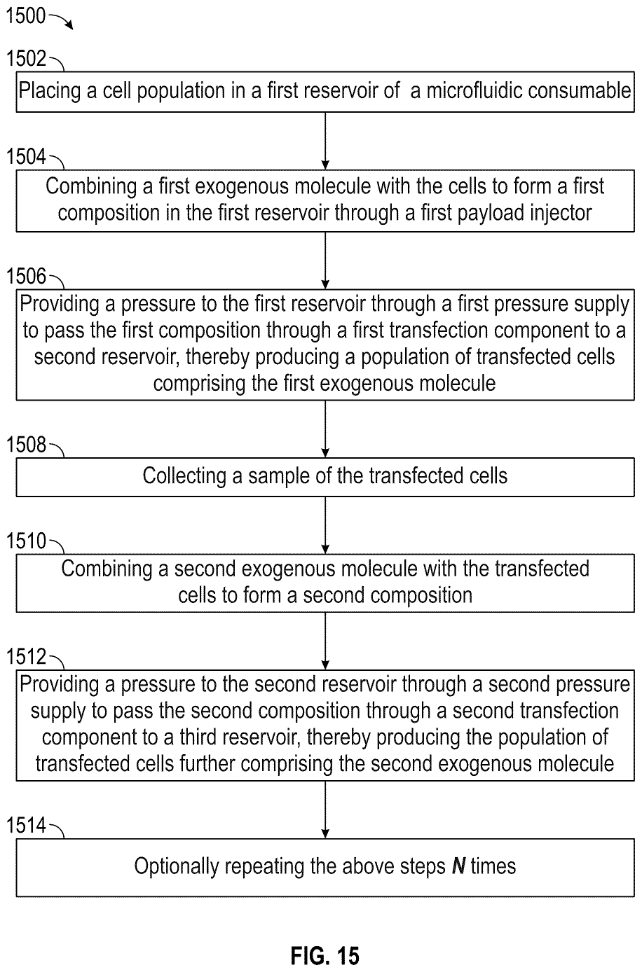

Further provided herein is a method for transfecting a plurality of cells with at least one exogenous molecule. The method 1500 of FIG. 15 may transfect a plurality of cells with a plurality of exogenous molecules sequentially. The method 1500 may be self-contained and capable of providing a sample after each transfection event. Further, the method 1500 may be operable to produce multiple transfection events much quicker than methods known in the art, while maintaining the same or better successful transfection rates and viability percentages. In some examples, the method 1500 is conducted using system 1400.

As illustrated in FIG. 15, at a first step 1502, the method 1500 may include placing a cell population (e.g., plurality of cells) in a first reservoir of a microfluidic consumable.

As illustrated in FIG. 15, at a second step 1504, the method 1500 may include combining a first exogenous molecule with the cells to form a first cell composition in the first reservoir. The first exogenous molecule may be moved from the payload reservoir into the first reservoir by providing a flow rate (e.g., via a pressure). The first exogenous molecule may be any of the exogenous molecules described herein.

As illustrated in FIG. 15, at a third step 1506, the method 1500 may include providing a pressure (e.g., through a first pressure supply) to the first reservoir to pass the first composition through a transfection component of a first microfluidic transfection device to a second reservoir. By passing the first composition through the transfection component, the plurality of cells may be transfected with the first exogenous molecule, producing a first population of transfected cells. In an example, the first microfluidic transfection device may be tuned or optimized for the first transfection event by increasing or decreasing the gap size or the other transfection parameters as described herein.

As illustrated in FIG. 15, at fourth step 1508, the method 1500 may include collecting a sample of transfected cells. In some examples, the sample may be collected by placing a second sample port valve in an open state and supplying a flow rate (e.g., via pressure from a second pressure supply) to the second reservoir to move a sample of cells through the sample port. In some examples, the sample may be removed while the cells are being held in the second reservoir for a holding period to allow sufficient recovery for the first population of transfected cells. In some examples, the hold period may be about 0.1 seconds to about 60 minutes.

As illustrated in FIG. 15, at a fifth step 1510, the method 1500 may include combining a second exogenous molecule with the first population of transfected cells to form a second composition. In some examples, the second payload reservoir may provide the second exogenous molecule to the second reservoir. In some examples, a flow rate (e.g., via a pressure) may be applied to the second payload reservoir to move the second exogenous molecule into the second reservoir. The second exogenous molecule may be any of the exogenous molecules described herein.

As illustrated in FIG. 15, at a sixth step 1512, the method 1500 may include providing a pressure to the second reservoir through a second pressure supply to pass the second composition through a transfection component of a second microfluidic transfection device to a third reservoir. By passing the second composition through the transfection component, the first population of transfected cells may be transfected with the second exogenous molecule, producing a second population of transfected cells. The second microfluidic transfection device may be optimized as described herein.

A sample may be removed from the third reservoir using a third pressure supply, placing a third sample port valve in an open state, and collecting the sample. In some examples, the sample may be removed while the second population of transfected cells is being held in a holding period to allow for sufficient recovery of the second population of transfected cells. In an example, the holding period may be about 0.1 seconds to about 60 minutes.

As illustrated in FIG. 15, at a seventh step 1514, the method 1500 may include repeating steps 1502-1508 N times. N may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some examples, the same exogenous molecule may be transfected into the cells N times. In other examples, a different exogenous molecule may be used for each transfection event. In further examples, the same exogenous molecule may be used in some transfection events and a different exogenous molecule may be used in other transfection events.

The systems and methods provided herein may be combined to produce different results using components of the different systems. For example, the system of FIG. 1A or FIG. 1B and the system of FIG. 4 may be combined in a sequential processing scheme by using the system 100 for first and second transfection events and system 400 for further transfection events. In other examples, the different systems may be arranged sequentially to provide optimal cell transfection results.

The individual components of the systems and methods provided herein may be rearranged. For example, every system may have a cell sorter. In another example, some systems may not need a second payload reservoir, as the first payload reservoir can be a removable payload and continue to provide new payloads (e.g., exogenous molecules). The systems and methods provided herein may include some or all of the components discussed herein. It will be appreciated that the described systems and methods are only examples. Components of one system may be combined with components of other systems to form systems not specifically described herein. For example, some components of one system may be combined with one or more components of another system to form a system not specifically described herein.

The systems and methods provided herein may not require an expansion step, meaning transfected cells may not require a long period of time between transfection events. Further, the systems and methods provided herein may be automated (e.g., at least one processor may control the flow rate supplies, the delivery of exogenous molecules, the sample ports, the valves, etc. without the need for manual user intervention). The systems and methods provided herein may be conducted in a self-contained environment. Self-contained means that the system is not open to the external environment, once the materials are in the systems, no contamination is introduced. The cells may be transfected sequentially without removal from the device, cassette, consumable, or system.

In an aspect, the methods may further comprise counting the cells and spinning them down to the necessary cell density to transfect the cells using the sequential processing methods disclosed herein. The methods may further include resuspending the cells in native media or any similar buffer and/or media. The methods may further include mixing a payload (e.g., exogenous molecule) with the resuspended cells in a container (e.g., microfuge tube) before placing the cells in a reservoir or in another part of the system. In some examples, the methods may include spinning the cells down to a necessary cell density after the first transfection event. In some examples, the methods may include resuspending the cells in fresh native media or any similar buffer or media after spinning them down to a necessary density a second time. The methods may further include mixing a payload (e.g., exogenous molecule) with the resuspended cells (e.g., first population of transfected cells). The methods may further include putting the cells back into an initial culture after transfecting them with the systems and methods disclosed herein. The expression of gene editing material may be observable within hours after transfecting a cell or cell using the systems and methods disclosed herein.

### EXAMPLES

### Example 1

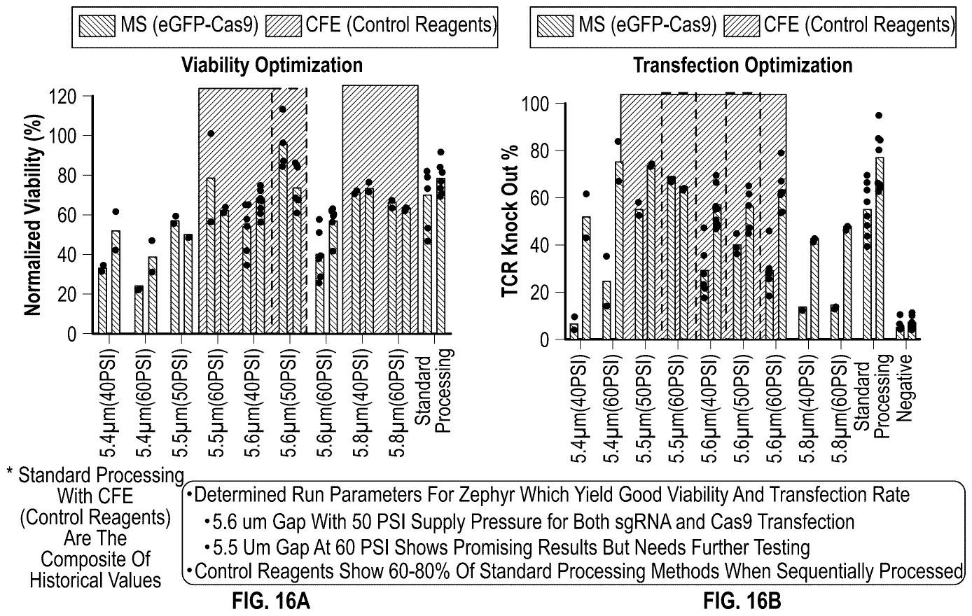

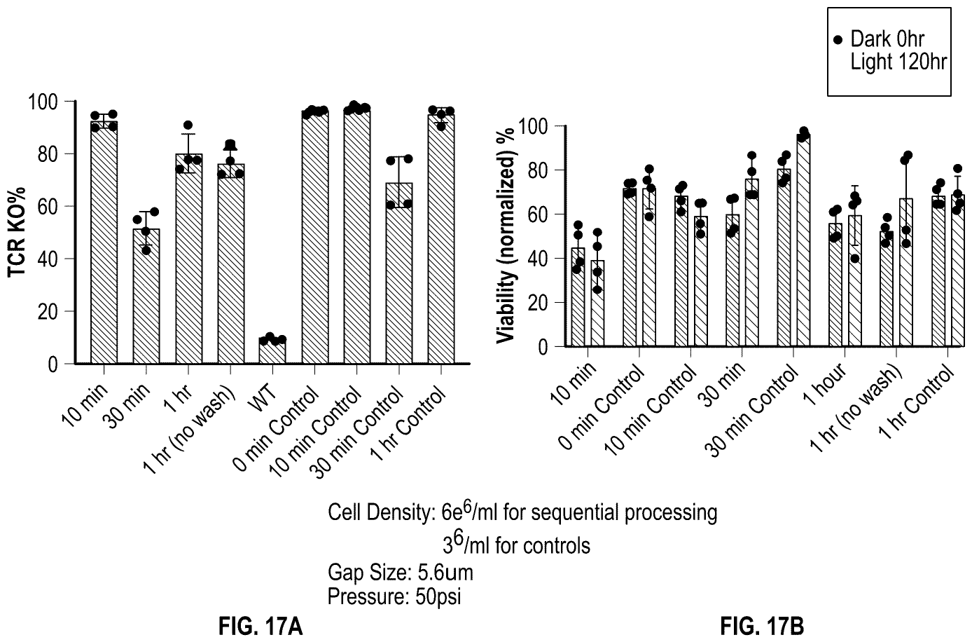

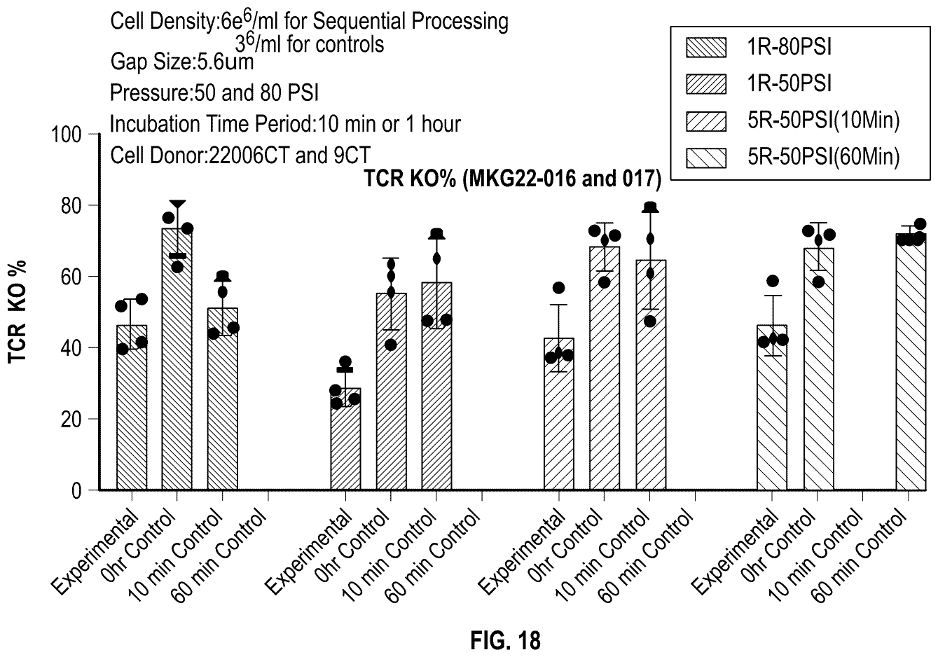

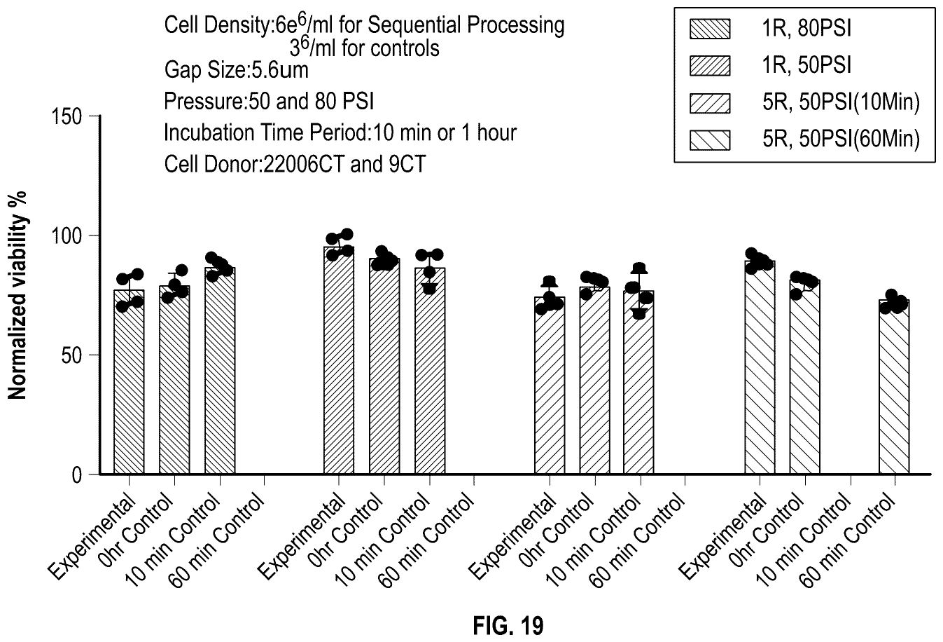

FIGS. 16A-19 show results testing system 100 using single-guide RNA (sgRNA) as the first payload (e.g., first exogenous molecule) and Cas9 as the second payload (e.g., second exogenous molecule). FIG. 16A shows the normalized viability as a function of the gap size of the microfluidic transfection devices and the pressure supplied. FIG. 16B shows the TCR knockout percentage as a function of the gap size of the microfluidic transfection device and the pressure supplied. FIG. 17A shows the TCR knockout percentage as a function of the holding period applied to the cells after the first transfection event. FIG. 17B shows the normalized viability percentage as a function of the holding period applied to the cells after the first transfection event. The results of FIGS. 17A-17B were experimentally observed with a supply pressure of 50 psi and a microfluidic transfection device gap size of 5.6 μm. FIG. 18 shows the TCR knockout percentage as a function of the holding period time wherein the gap size was 5.6 μm and the pressure supplied was 50 psi. FIG. 19 shows the normalized viability percentage as a function of control time, wherein the experiment was run at pressures of 50 psi and 80 psi with a gap size of 5.6 μm.

The foregoing are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

### Example 2

### Cell Viability and Transfection Efficiency After Sequential Editing Were examined in activated T cells.

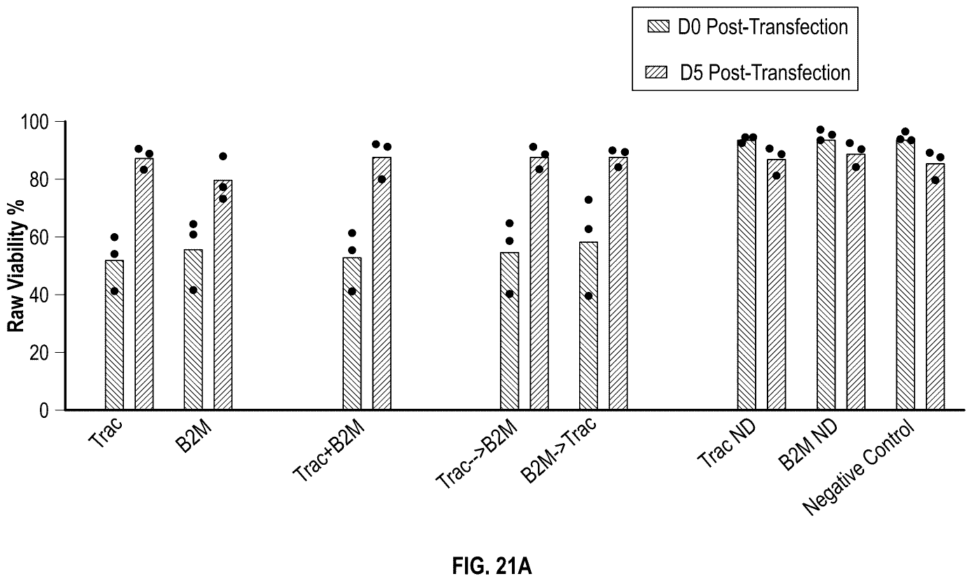

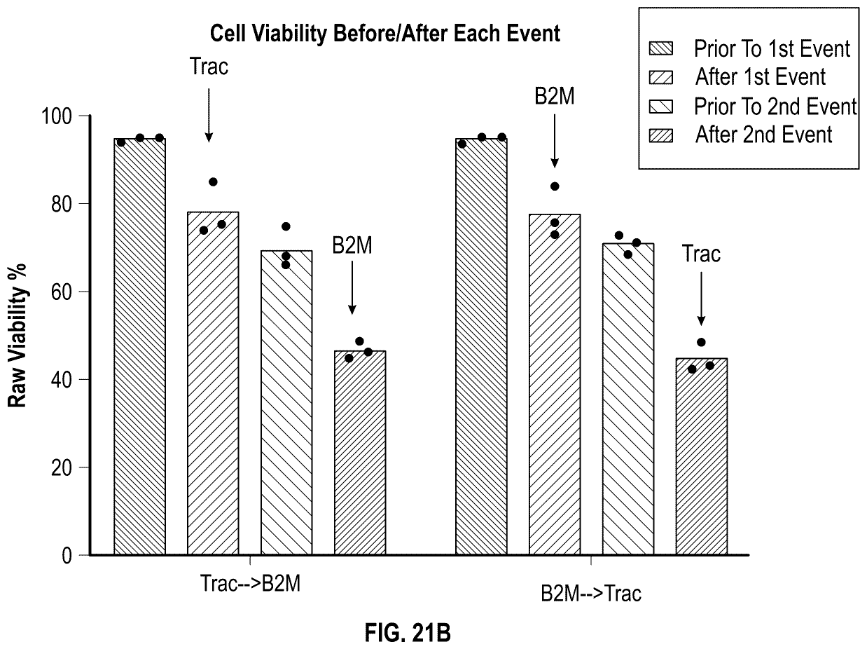

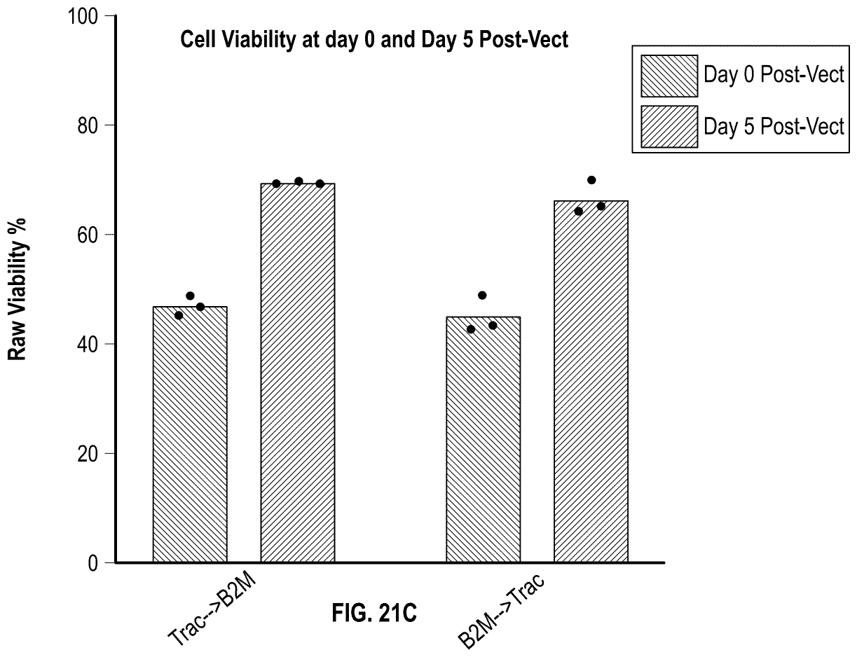

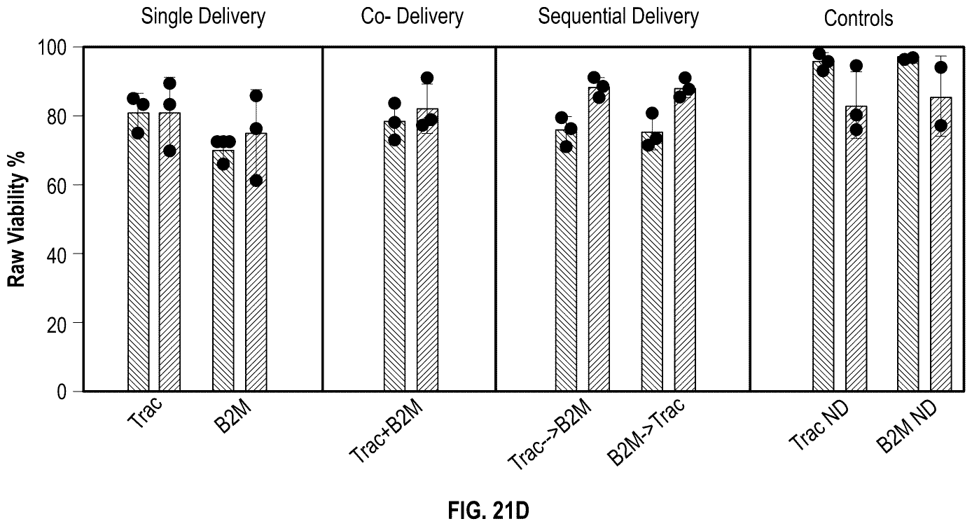

T-cells were transfected using the method described above. Editing was performed to knock out endogenous TRAC and B2M by sequential delivery of Cas9 RNP with a 1 hour interval, where cells are held in culture. Post-transfected T cells were subjected to automatic cell counter using NucleoCounter NC-3000 (chemometec Inc.) to measure live or dead cell number (AO/DAPI staining) and calculate the percentage of viability. T cell activation was performed for 48 hours. Cell viability was compared to single knock outs of TRAC or B2M, or simultaneous knockouts. Further a no device (“ND”) was used as a negative control with cell sample prepared in the same batch as experimental group but were not transfected. As shown in FIG. 21A, cell viability remained consistent between the edited T cells. Further, cell viability after each editing event was examined. Sequential editing was performed at 24 hour interval where cells are held in culture, and T cells were activated for 24 hours. Cell viability of the edited T cells were comparable (FIG. 21B). Viability of cells were found to be enhanced at five days after transfection, as compared to day 0 (FIG. 21C). The viability of cells examined after single delivery of either TRAC or B2M editing Cas9 RNP, co-delivery of TRAC and B2M editing Cas9 RNP, sequential delivery of TRAC and B2M Cas9 RNP with a 24 hour interval where cells are held in culture, and with 6 hours of T cell activation were found to be similar between the different groups at 0 days and 5 days after transfection, as compared to cells from cell sample prepared in the same batch as experimental group but were not transfected (“ND”) (FIG. 21D).

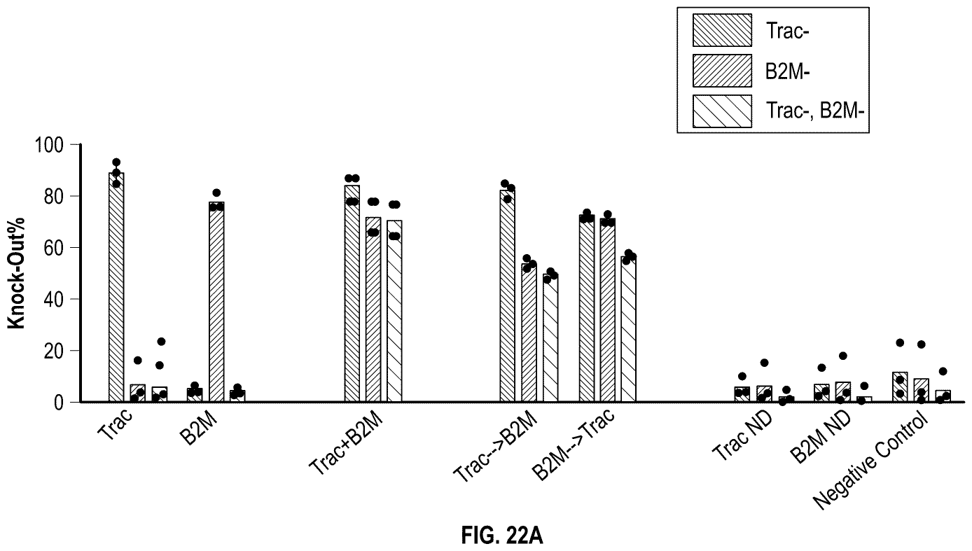

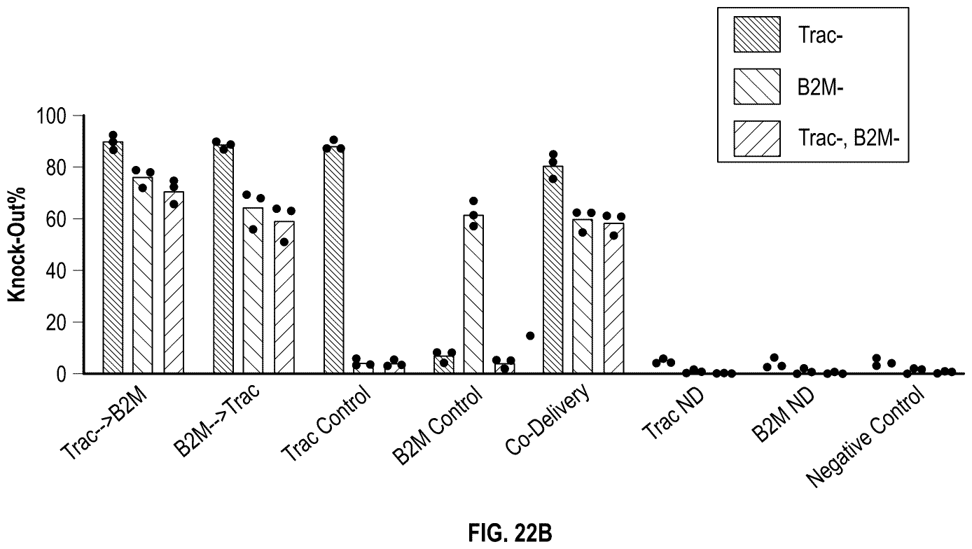

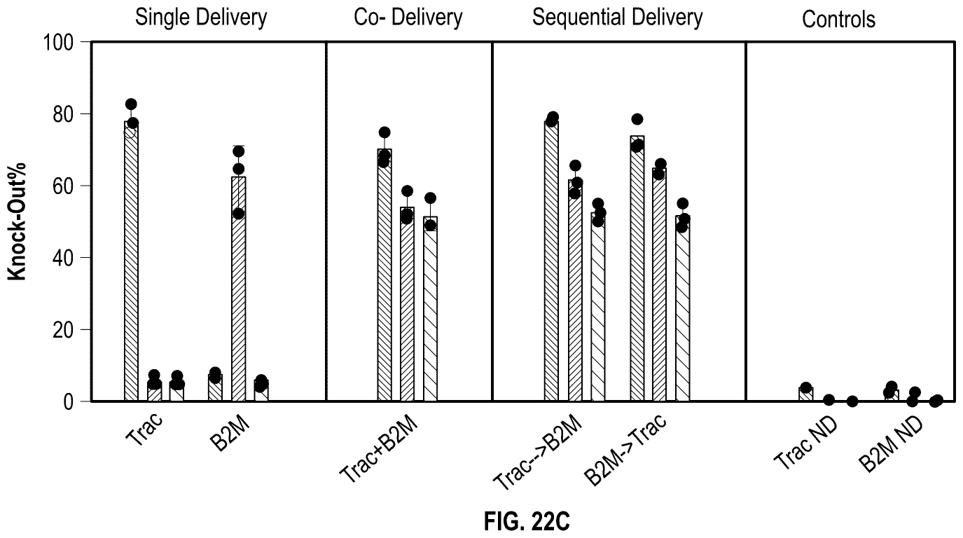

Transfection efficiency was additionally examined. Editing was performed to knock out TRAC and B2M by sequential delivery of Cas9 RNP at 1 hour interval. Post-transfected T-cells were cultured to the indicated time and subjected to flow cytometry (CytoFlex S, Beckman Coulter) analysis by staining the targeting marker on cell surface or detecting the reporter signal. T cell activation was performed for 48 hours. Results were reported as a percentage to the control groups and data showed as average of biological replicates. Transfection efficiency was measured as the percentage of cells which didn't express the target protein. Percentage of cells with knock-out was found to be high with sequential editing indicating that the transfection efficiency remined high after sequential editing (FIG. 22A). Sequential editing of TRAC and B2M in T cells with a 24 hour interval, wherein the cells are held in culture and a 24 hour T cell activation (1^{st} event) exhibited high transfection efficiency similar to single editing of TRAC or B2M or co-delivery of TRAC and B2M (FIG. 22B). FIG. 22C further shows that cells with sequential delivery of TRAC and B2M editing Cas9 RNP with 24 hour interval where cells are held in culture and 6 hour T cell activation exhibited high percentage of knock-out as compared single delivery of TRAC or B2M Cas9-RNP and no device (ND) controls, and similar knock out percentages as co-delivery of TRAC and B2M editing Cas9 RNP.

Translocation events after sequential delivery of Cas9 RNP was examined. Frequency of translocation in T cells with sequential delivery of Cas9 RNP to knock out TRAC and PD1, or co-delivery of Cas9 RNP to knock out TRAC and PD1, was measured using chromosomal translocation assay by qPCR. T cells were activated for 6 hours after first event. Sequential editing had a 24 hour interval where cells are held in culture between delivery. Compared to co-delivery, sequential delivery reduced translocation event by about 75%.

1. A method for high throughput introduction of an exogenous molecule into cells in a population of cells which are heterogeneous, the method comprising:
(a) obtaining or having obtained the population of cells sorted into at least a first sub-population and a second sub-population of cells, wherein the first sub-population and second sub-population differ in size, cell membrane stiffness, FACS characteristics, and/or other physical characteristics;
(b) combining the first sub-population of cells in an initial fluid medium with the exogenous molecule to form a first composition;
(c) combining the second sub-population of cells in the initial fluid medium with the exogenous molecule thereby forming a second composition;
(d) passing the first composition through a microfluidic transfection device using a first set of process parameters, thereby introducing the exogenous molecule into the cells in the first sub-population to form a first population of transfected cells comprising the exogenous molecule;
(e) passing the second composition through a microfluidic transfection device using a second set of process parameters, thereby introducing the exogenous molecule to the cells in the second sub-population to form a second population of transfected cells comprising the exogenous molecule; and
(f) optionally observing a hold period.

2. The method of claim 1, wherein (a)-(f) comprise a microfluidic processing cycle, and the method comprises passing the cells through N microfluidic processing cycles wherein N=2 to 10.

3. The method of claim 1, wherein obtaining the population of cells sorted into at least a first sub-population and a second sub-population of cells comprises passing the heterogenous cell population through a cell sorter, thereby producing at least a first sub-population comprising cells each having a cell diameter below a first diameter cut-off value, and a second sub-population of larger cells each having a diameter above the first diameter cut-off value and below a second diameter cut-off value.

4. The method of claim 1, wherein obtaining the population of cells sorted into at least the first sub-population and the second sub-population comprises passing the heterogenous cell population through a cell sorter, thereby producing at least the first sub-population comprising cells having a first set of FACS characteristics, and a second sub-population of comprising cells having a second set of FACS characteristics.

5. The method of claim 4, wherein the first set and second set of FACS characteristics comprise different phenotypes.

6. The method of claim 4, wherein the first set and second set of FACS characteristics comprise differing receptor types.

7. The method of claim 4, wherein the first set and second set of FACS characteristics comprise differing frequencies.

8. The method of claim 1, wherein obtaining the population of cells sorted into at least a first sub-population and a second sub-population of cells comprises passing the heterogenous cell population through a cell sorter, thereby producing at least a first sub-population comprising cells each having a cell stiffness below a first stiffness cut-off value, and a second sub-population comprising cells each having a stiffness above the first stiffness cut-off value and below a second stiffness cut-off value.

9. The method of claim 1, wherein obtaining the population of cells sorted into at least a first sub-population and a second sub-population comprises obtaining the cells sorted into N additional sub-populations by sorting cells into groups defined by additional ranges of cell diameters.

10. The method of claim 9, further comprising combining the first and the second up to the Nth sub-populations of transfected cells to form a heterogenous cell product.

11. The method of claim 1, wherein the heterogenous cell population comprises whole blood, wherein sorting the cells comprises sorting the cells into at least a first sub-population having an average cell diameter of 10-12 μm (neutrophils), a second sub-population having an average cell diameter of 12-15 μm (lymphocytes), and a third sub-population having an average cell diameter of 15-30 μm (monocytes).

12. The method of claim 1, wherein the exogenous molecule is selected from gene editing materials, nanoparticles, protein, antigens, amino-acids, viruses or viral components, DNA and related material, RNA and related material, lipids and related material, small molecules, and/or salts.

13. The method of claim 1, wherein the exogenous molecule comprises a nucleotide sequence encoding an amino acid sequence of interest, the method further comprising maintaining the combined cell populations in a holding period for a time and conditions sufficient for the cells to express one or more nucleotide sequences.

14. The method of claim 1, wherein the hold period is about 0.1 seconds to about 60 minutes.

15. A method for high throughput introduction of a plurality of exogenous molecules to a plurality of cells comprising a heterogenous cell population, the method comprising:
(a) obtaining the population of cells sorted into at least a first sub-population and a second sub-population of cells, wherein the first sub-population and second sub-population differ in size and/or cell stiffness and/or FACS characteristics;
(b) combining a first exogenous molecule with the first sub-population of cells in an initial fluid medium to form a first cell composition;
(c) passing the first cell composition through a microfluidic transfection device, thereby introducing the first exogenous molecule into the cells to form a first population of transfected cells comprising the first exogenous molecule;
(d) combining a second exogenous molecule and optionally the first exogenous molecule with the second sub-population of cells in the initial fluid medium to form a second cell composition;
(e) passing the second cell composition through a microfluidic transfection device, thereby introducing the second exogenous molecule and optionally the first exogenous molecule into the cells to form a second population of transfected cells comprising the second exogenous molecule and optionally the first exogenous molecule; and
(f) optionally repeating (b)-(c) N times with the first exogenous molecule, the second exogenous molecule, and/or N additional exogenous molecules to form N additional populations of transfected cells, each additional population transfected with the first exogenous molecule, the second exogenous molecule, and/or an additional exogenous molecule, wherein the additional exogenous molecules may be the same exogenous molecule or a different exogenous molecule from the first exogenous molecule and the second exogenous molecule.

16. The method of claim 15, further comprising optionally holding the cells for a holding period before optionally repeating (b)-(c).

17. The method of claim 15, wherein the FACS characteristics are one or more of phenotype, receptor type, and/or frequency.

18. The method of claim 17, further comprising tuning the microfluidic transfection device prior to passing the cell compositions through the microfluidic transfection device.

19. The method of claim 18, wherein the microfluidic transfection device is tuned based on the sub-population size, cell stiffness, or FACS characteristics.

20. The method of claim 19, wherein tuning the microfluidic transfection device comprises increasing or decreasing a gap size of the microfluidic transfection device.
